SEQUENCE LISTING (185 sequences)

SEQ ID NO: 1 - protein, 450 aa, synthetic construct
  source 1..450: note = Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
  source 1..214: note = Trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, synthetic construct
  source 1..449: note = Cetuximab heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
  source 1..214: note = Cetuximab light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 451 aa, synthetic construct
  source 1..451: note = Rituximab heavy chain chimeric
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 213 aa, synthetic construct
  source 1..213: note = Rituximab light chain chimeric
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 452 aa, synthetic construct
  source 1..452: note = Daratumumab heavy chain
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, synthetic construct
  source 1..214: note = Daratumumab light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, synthetic construct
  source 1..450: note = Avelumab heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, synthetic construct
  source 1..216: note = Avelumab light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 11 - protein, 1057 aa, synthetic construct
  source 1..1057: note = Trastuzumab HC1-41BBLT fusion
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 12 - protein, 588 aa, synthetic construct
  source 1..588: note = Trastuzumab HC2-IL21 fusion
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - protein, 648 aa, synthetic construct
  source 1..648: note = Trastuzumab HC1-41BBL fusion
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEAARHAWQLTQGATVLGLFRVTPEIPAGLPSPRSE

SEQ ID NO: 14 - protein, 450 aa, synthetic construct
  source 1..450: note = Trastuzumab HC1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 450 aa, synthetic construct
  source 1..450: note = Trastuzumab HC2
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 451 aa, synthetic construct
  source 1..451: note = Anti-NKG2D HC1
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 17 - protein, 475 aa, synthetic construct
  source 1..475: note = Trastuzumab-scFv-HC2
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 18 - protein, 1057 aa, synthetic construct
  source 1..1057: note = Trastuzumab HC-41BBLT fusion
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 19 - protein, 588 aa, synthetic construct
  source 1..588: note = Trastuzumab HC-IL21 fusion
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 20 - protein, 210 aa, synthetic construct
  source 1..210: note = Anti-NKG2D LC
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 352 aa, synthetic construct
  source 1..352: note = Trastuzumab LC-IL21 fusion
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 22 - protein, 98 aa, synthetic construct
  source 1..98: note = VH3/3-11
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 23 - protein, 330 aa, synthetic construct [sequence omitted]

SEQ ID NO: 24 - protein, 5 aa, synthetic construct
  source 1..5: note = Trastuzumab CDR-H1
DTYIH

SEQ ID NO: 25 - protein, 17 aa, synthetic construct
  source 1..17: note = Trastuzumab CDR-H2
RIYPTNGYTRYADSVKG

SEQ ID NO: 26 - protein, 10 aa, synthetic construct
  source 1..10: note = Trastuzumab CDR-H3
GGDGFYAMDY

SEQ ID NO: 27 - protein, 11 aa, synthetic construct
  source 1..11: note = Trastuzumab CDR-L1
RASQDVNTAVA

SEQ ID NO: 28 - protein, 7 aa, synthetic construct
  source 1..7: note = Trastuzumab CDR-L2
SASFLYS

SEQ ID NO: 29 - protein, 9 aa, synthetic construct
  source 1..9: note = Trastuzumab CDR-L3
QQHYTTPPT

SEQ ID NO: 30 - protein, 5 aa, synthetic construct
  source 1..5: note = flexible linker
GGGGS

SEQ ID NO: 31 - protein, 20 aa, synthetic construct
  source 1..20: note = flexible linker
GGGGSGGGGSGGGGSGGGGS

SEQ ID NO: 32 - protein, 7 aa, synthetic construct
  source 1..7: note = flexible linker
GGGSGGG

SEQ ID NO: 33 - protein, 10 aa, synthetic construct
  source 1..10: note = flexible linker
GGSGGGGSGG

SEQ ID NO: 34 - protein, 18 aa, synthetic construct
  source 1..18: note = flexible linker
KESGSVSSEQLAQFRSLD

SEQ ID NO: 35 - protein, 14 aa, synthetic construct
  source 1..14: note = flexible linker
EGKSSGSGSESKST

SEQ ID NO: 36 - protein, 592 aa, synthetic construct
  source 1..592: note = 4-1BBL trimer
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 37 - protein, 184 aa, synthetic construct
  source 1..184: note = 4-1BBL monomer
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 38 - protein, 133 aa, synthetic construct [sequence omitted]

SEQ ID NO: 39 - protein, 120 aa, synthetic construct [sequence omitted]

SEQ ID NO: 40 - protein, 108 aa, synthetic construct
  source 1..108: note = VL Trastuzumab
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 41 - protein, 119 aa, synthetic construct
  source 1..119: note = VH Cetuximab
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 42 - protein, 108 aa, synthetic construct
  source 1..108: note = VL Cetuximab
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 43 - protein, 121 aa, synthetic construct [sequence omitted]

SEQ ID NO: 44 - protein, 107 aa, synthetic construct
  source 1..107: note = VL Rituximab
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 45 - protein, 122 aa, synthetic construct [sequence omitted]

SEQ ID NO: 46 - protein, 108 aa, synthetic construct
  source 1..108: note = VL Daratumumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKR

SEQ ID NO: 47 - protein, 120 aa, synthetic construct
  source 1..120: note = VH Avelumab
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 48 - protein, 112 aa, synthetic construct
  source 1..112: note = VL Avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQ

SEQ ID NO: 49 - protein, 96 aa, synthetic construct
  source 1..96: note = VK3/A27
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 50 - protein, 304 aa, synthetic construct
  source 1..304: note = NKp46 NP_004820
MSSTLPALLCVGLCLSQRISAQQQTLPKPFIWAEPHFMVPKEKQVTICCQGNYGAVEYQLHFEGSLFAVDRPKPPERINKVQFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 51 - protein, 276 aa, synthetic construct
  source 1..276: note = NKp44 NP_004819
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 52 - protein, 201 aa, synthetic construct
  source 1..201: note = NKp30 NP_667341
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 53 - protein, 216 aa, synthetic construct
  source 1..216: note = NKG2D NP_001186734
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 54 - protein, 336 aa, synthetic construct
  source 1..336: note = DNAM-1 NP_006557
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 55 - protein, 254 aa, synthetic construct
  source 1..254: note = CD16A P08637
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 56 - protein, 335 aa, synthetic construct
  source 1..335: note = SLAMF7 Q9NQ25-1
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 57 - protein, 450 aa, synthetic construct
  source 1..450: note = anti-NKp46 heavy chain
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
  source 1..214: note = anti-NKp46 light chain
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 448 aa, synthetic construct
  source 1..448: note = Atezolizumab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 214 aa, synthetic construct
  source 1..214: note = Atezolizumab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 451 aa, synthetic construct
  source 1..451: note = Durvalumab heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 215 aa, synthetic construct
  source 1..215: note = Durvalumab light chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 450 aa, synthetic construct
  source 1..450: note = Cosibelimab heavy chain
EVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 218 aa, synthetic construct
  source 1..218: note = Cosibelimab light chain
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - protein, 450 aa, synthetic construct
  source 1..450: note = Margetuximab heavy chain
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 66 - protein, 214 aa, synthetic construct
  source 1..214: note = Margetuximab light chain
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 448 aa, synthetic construct
  source 1..448: note = Pertuzumab heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 214 aa, synthetic construct
  source 1..214: note = Pertuzumab light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 452 aa, synthetic construct
  source 1..452: note = Enoblituzumab heavy chain
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 70 - protein, 214 aa, synthetic construct
  source 1..214: note = Enoblituzumab light chain
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 451 aa, synthetic construct
  source 1..451: note = Necitumumab heavy chain
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 214 aa, synthetic construct
  source 1..214: note = Necitumumab light chain
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 445 aa, synthetic construct
  source 1..445: note = Panitumumab heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 74 - protein, 214 aa, synthetic construct
  source 1..214: note = Panitumumab light chain
DIQMTQSPSSLSASVGDRVTITCOASODISNYLNWYQQKPGKAPKLLIYDASNLETGVPSRFSGSGSGTDFTFTISSLQPEDIATYFCQHFDHLPIAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 455 aa, synthetic construct
  source 1..455: note = Amivantamab EGFR-binding heavy chain
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 76 - protein, 214 aa, synthetic construct
  source 1..214: note = Amivantamab EGFR-binding light chain
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 449 aa, synthetic construct
  source 1..449: note = Amivantamab cMet-binding heavy chain
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 78 - protein, 214 aa, synthetic construct
  source 1..214: note = Amivantamab cMet-binding light chain
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 448 aa, synthetic construct
  source 1..448: note = Zolbetuximab heavy chain
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 220 aa, synthetic construct
  source 1..220: note = Zolbetuximab light chain
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 443 aa, synthetic construct
  source 1..443: note = Dinutuximab heavy chain
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 220 aa, synthetic construct
  source 1..220: note = Dinutuximab light chain
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 449 aa, synthetic construct
  source 1..449: note = Naxitamab heavy chain
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 211 aa, synthetic construct
  source 1..211: note = Naxitamab light chain
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 447 aa, synthetic construct
  source 1..447: note = Enfortumab heavy chain
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 214 aa, synthetic construct
  source 1..214: note = Enfortumab light chain
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 449 aa, synthetic construct
  source 1..449: note = Farletuzumab heavy chain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 217 aa, synthetic construct
  source 1..217: note = Farletuzumab light chain
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 448 aa, synthetic construct
  source 1..448: note = Tisotumab heavy chain
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 214 aa, synthetic construct
  source 1..214: note = Tisotumab light chain
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 447 aa, synthetic construct
  source 1..447: note = Mirvetuximab heavy chain
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 218 aa, synthetic construct
  source 1..218: note = Mirvetuximab light chain
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 451 aa, synthetic construct
  source 1..451: note = Sacituzumab heavy chain
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 214 aa, synthetic construct
  source 1..214: note = Sacituzumab light chain
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 446 aa, synthetic construct
  source 1..446: note = Vobramitamab heavy chain
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 215 aa, synthetic construct
  source 1..215: note = Vobramitamab light chain
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 449 aa, synthetic construct
  source 1..449: note = Onartuzumab heavy chain
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 98 - protein, 220 aa, synthetic construct
  source 1..220: note = Onartuzumab light chain
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 227 aa, synthetic construct
  source 1..227: note = Onartuzumab Fc chain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 457 aa, synthetic construct
  source 1..457: note = Olaratumab heavy chain
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 214 aa, synthetic construct
  source 1..214: note = Olaratumab light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 447 aa, synthetic construct
  source 1..447: note = Rovalpituzumab heavy chain
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 214 aa, synthetic construct
  source 1..214: note = Rovalpituzumab light chain
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 118 aa, synthetic construct [sequence omitted]

SEQ ID NO: 105 - protein, 108 aa, synthetic construct
  source 1..108: note = VL Atezolizumab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 106 - protein, 121 aa, synthetic construct
  source 1..121: note = VH Durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 107 - protein, 109 aa, synthetic construct
  source 1..109: note = VL Durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKR

SEQ ID NO: 108 - protein, 120 aa, synthetic construct
  source 1..120: note = VH Cosibelimab
EVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSS

SEQ ID NO: 109 - protein, 112 aa, synthetic construct
  source 1..112: note = VL Cosibelimab
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVL

SEQ ID NO: 110 - protein, 120 aa, synthetic construct
  source 1..120: note = VH Margetuximab
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 111 - protein, 108 aa, synthetic construct
  source 1..108: note = VL Margetuximab
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKR

SEQ ID NO: 112 - protein, 119 aa, synthetic construct [sequence omitted]

SEQ ID NO: 113 - protein, 108 aa, synthetic construct
  source 1..108: note = VL Pertuzumab
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 114 - protein, 122 aa, synthetic construct
  source 1..122: note = VH Enoblituzumab
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 115 - protein, 108 aa, synthetic construct
  source 1..108: note = VL Enoblituzumab
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKR

SEQ ID NO: 116 - protein, 121 aa, synthetic construct [sequence omitted]

SEQ ID NO: 117 - protein, 108 aa, synthetic construct
  source 1..108: note = VL Necitumumab
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKR

SEQ ID NO: 118 - protein, 119 aa, synthetic construct [sequence omitted]

SEQ ID NO: 119 - protein, 108 aa, synthetic construct
  source 1..108: note = VL Panitumumab
DIQMTQSPSSLSASVGDRVTITCOASODISNYLNWYQQKPGKAPKLLIYDASNLETGVPSRFSGSGSGTDFTFTISSLQPEDIATYFCQHFDHLPIAFGGGTKVEIKR

SEQ ID NO: 120 - protein, 125 aa, synthetic construct [sequence omitted]

SEQ ID NO: 121 - protein, 107 aa, synthetic construct [sequence omitted]

SEQ ID NO: 122 - protein, 119 aa, synthetic construct
  source 1..119: note = VH Amivantamab cMet-binding
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 123 - protein, 107 aa, synthetic construct
  source 1..107: note = VL Amivantamab cMet-binding
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 124 - protein, 118 aa, synthetic construct
  source 1..118: note = VH Zolbetuximab
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 125 - protein, 114 aa, synthetic construct
  source 1..114: note = VL Zolbetuximab
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKR

SEQ ID NO: 126 - protein, 113 aa, synthetic construct [sequence omitted]

SEQ ID NO: 127 - protein, 114 aa, synthetic construct
  source 1..114: note = VL Dinutuximab
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKR

SEQ ID NO: 128 - protein, 119 aa, synthetic construct
  source 1..119: note = VH Naxitamab
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSS

SEQ ID NO: 129 - protein, 105 aa, synthetic construct
  source 1..105: note = VL Naxitamab
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKR

SEQ ID NO: 130 - protein, 117 aa, synthetic construct
  source 1..117: note = VH Enfortumab
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 131 - protein, 108 aa, synthetic construct
  source 1..108: note = VL Enfortumab
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR

SEQ ID NO: 132 - protein, 119 aa, synthetic construct
  source 1..119: note = VH Farletuzumab
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 133 - protein, 111 aa, synthetic construct
  source 1..111: note = VL Farletuzumab
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKR

SEQ ID NO: 134 - protein, 118 aa, synthetic construct
  source 1..118: note = VH Tisotumab
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 135 - protein, 108 aa, synthetic construct
  source 1..108: note = VL Tisotumab
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKR

SEQ ID NO: 136 - protein, 118 aa, synthetic construct
  source 1..118: note = VH Mirvetuximab
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 137 - protein, 112 aa, synthetic construct
  source 1..112: note = VL Mirvetuximab
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 138 - protein, 121 aa, synthetic construct
  source 1..121: note = VH Sacituzumab
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 139 - protein, 108 aa, synthetic construct
  source 1..108: note = VL Sacituzumab
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 140 - protein, 117 aa, synthetic construct
  source 1..117: note = VH Vobramitamab
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 141 - protein, 109 aa, synthetic construct
  source 1..109: note = VL Vobramitamab
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKR

SEQ ID NO: 142 - protein, 119 aa, synthetic construct [sequence omitted]

SEQ ID NO: 143 - protein, 114 aa, synthetic construct
  source 1..114: note = VL Onartuzumab
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 144 - protein, 124 aa, synthetic construct
  source 1..124: note = VH Sibrotuzumab
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 145 - protein, 113 aa, synthetic construct
  source 1..113: note = VL Sibrotuzumab
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 146 - protein, 127 aa, synthetic construct
  source 1..127: note = VH Olaratumab
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSS

SEQ ID NO: 147 - protein, 108 aa, synthetic construct
  source 1..108: note = VL Olaratumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIKR

SEQ ID NO: 148 - protein, 118 aa, synthetic construct
  source 1..118: note = VH Rovalpituzumab
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 149 - protein, 108 aa, synthetic construct
  source 1..108: note = VL Rovalpituzumab
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKR

SEQ ID NO: 150 - protein, 10 aa, synthetic construct
  source 1..10: note = Atezolizumab CDR-H1
GFTFSDSWIH

SEQ ID NO: 151 - protein, 18 aa, synthetic construct
  source 1..18: note = Atezolizumab CDR-H2
AWISPYGGSTYYADSVKG

SEQ ID NO: 152 - protein, 9 aa, synthetic construct
  source 1..9: note = Atezolizumab CDR-H3
RHWPGGFDY

SEQ ID NO: 153 - protein, 11 aa, synthetic construct
  source 1..11: note = Atezolizumab CDR-L1
RASQDVSTAVA

SEQ ID NO: 154 - protein, 7 aa, synthetic construct
  source 1..7: note = Atezolizumab CDR-L2
SASFLYS

SEQ ID NO: 155 - protein, 9 aa, synthetic construct
  source 1..9: note = Atezolizumab CDR-L3
QQYLYHPAT

SEQ ID NO: 156 - protein, 5 aa, synthetic construct
  source 1..5: note = Avelumab CDR-H1
SYIMM

SEQ ID NO: 157 - protein, 17 aa, synthetic construct
  source 1..17: note = Avelumab CDR-H2
SIYPSGGITFYADTVKG

SEQ ID NO: 158 - protein, 11 aa, synthetic construct
  source 1..11: note = Avelumab CDR-H3
IKLGTVTTVDY

SEQ ID NO: 159 - protein, 14 aa, synthetic construct
  source 1..14: note = Avelumab CDR-L1
TGTSSDVGGYNYVS

SEQ ID NO: 160 - protein, 7 aa, synthetic construct
  source 1..7: note = Avelumab CDR-L2
DVSNRPS

SEQ ID NO: 161 - protein, 10 aa, synthetic construct
  source 1..10: note = Avelumab CDR-L3
SSYTSSSTRV

SEQ ID NO: 162 - protein, 10 aa, synthetic construct
  source 1..10: note = Durvalumab CDR-H1
GFTFSRYWMS

SEQ ID NO: 163 - protein, 17 aa, synthetic construct
  source 1..17: note = Durvalumab CDR-H2
NIKQDGSEKYYVDSVKG

SEQ ID NO: 164 - protein, 12 aa, synthetic construct
  source 1..12: note = Durvalumab CDR-H3
EGGWFGELAFDY

SEQ ID NO: 165 - protein, 12 aa, synthetic construct
  source 1..12: note = Durvalumab CDR-L1
RASQRVSSSYLA

SEQ ID NO: 166 - protein, 7 aa, synthetic construct
  source 1..7: note = Durvalumab CDR-L2
DASSRAT

SEQ ID NO: 167 - protein, 9 aa, synthetic construct
  source 1..9: note = Durvalumab CDR-L3
QQYGSLPWT

SEQ ID NO: 168 - protein, 1058 aa, synthetic construct
  source 1..1058: note = AVC-016 HC1
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 169 - protein, 589 aa, synthetic construct
  source 1..589: note = AVC-016 HC2
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 170 - protein, 214 aa, synthetic construct; identical to SEQ ID NO: 94
  source 1..214: note = AVC-016 LC

SEQ ID NO: 171 - protein, 1050 aa, synthetic construct
  source 1..1050: note = AVC_017 HC1
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 172 - protein, 581 aa, synthetic construct
  source 1..581: note = AVC-017 HC2
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 173 - protein, 220 aa, synthetic construct; identical to SEQ ID NO: 82
  source 1..220: note = AVC-017 LC

SEQ ID NO: 174 - protein, 1055 aa, synthetic construct
  source 1..1055: note = AVC-018 HC1
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 175 - protein, 586 aa, synthetic construct
  source 1..586: note = AVC-018 HC2
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 176 - protein, 218 aa, synthetic construct; identical to SEQ ID NO: 92
  source 1..218: note = AVC-018 LC

SEQ ID NO: 177 - protein, 1055 aa, synthetic construct
  source 1..1055: note = AVC-019 HC1
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 178 - protein, 586 aa, synthetic construct
  source 1..586: note = AVC-019 HC2
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 179 - protein, 214 aa, synthetic construct
  source 1..214: note = AVC-019 LC
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 1056 aa, synthetic construct
  source 1..1056: note = AVC-020 HC1
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 181 - protein, 587 aa, synthetic construct
  source 1..587: note = AVC-020 HC2
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 182 - protein, 214 aa, synthetic construct; identical to SEQ ID NO: 4
  source 1..214: note = AVC-020 LC

SEQ ID NO: 183 - protein, 1057 aa, synthetic construct
  source 1..1057: note = AVC-021 HC1
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 184 - protein, 588 aa, synthetic construct
  source 1..588: note = AVC-021 HC2
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 185 - protein, 216 aa, synthetic construct; identical to SEQ ID NO: 10
  source 1..216: note = AVC-021 LC

(19) United States
(12) Patent Application Publication
(10) Pub. No.: US 2026/0103539 A1
(43) Pub. Date: Apr. 16, 2026

(54) Multispecific antigen binding proteins for tumor-targeting of NK cells and use thereof

(71) Applicant: Avidicure IP B.V., Amsterdam (NL)

(72) Inventor: Robert Heinz Edward Friesen, Breukelen (NL)

(73) Assignee: Avidicure IP B.V., Amsterdam (NL)

(21) Appl. No.: 19/111,337

(22) PCT Filed: Sep. 15, 2023
(86) PCT No.: PCT/EP2023/075441

§ 371 Date: Mar. 13, 2025

(30) Foreign Application Priority Data
Sep. 15, 2022 (EP) 22195902.6
Sep. 15, 2022 (EP) 22195904.2

(51) Int. Cl. C07K 16/30; A61K 35/17; A61K 40/11; A61K 40/15; A61P 35/00; C07K 14/54; C07K 14/705; C07K 19/00; C12N 5/0783

(52) U.S. Cl. CPC C07K 16/30; A61K 35/17; A61K 40/11; A61K 40/15; A61P 35/00; C07K 14/54; C07K 14/70596; C07K 19/00; C12N 5/0646; C07K 2317/31; C07K 2317/52; C07K 2317/55; C12N 2502/30

(57) ABSTRACT

The present invention relates to multispecific antigen binding proteins that comprise antigen-binding regions specific for a tumor-associated antigen (TAA) and NK cell-activating cytokines. The NK cell-activating cytokine preferably is at least one of an interleukin 21 receptor (IL21R) agonist and a 4-1BB agonist. The multispecific antigen binding protein can further comprise an antigen-binding region that has affinity for a surface antigen expressed on NK cells, e.g CD16A. The multispecific antigen binding proteins of the invention specifically redirect and activate NK cells to lyse targeted tumor cells. The invention further relates to the use of such multispecific antigen binding proteins in the treatment of cancer, preferably a cancer expressing the TAA.

### FIELD OF THE INVENTION

The present invention relates to the field of medicine, in particular to the fields of oncology, immunology and immunotherapy of tumors. Specifically, the invention relates to multispecific antigen binding proteins that specifically redirect and activate NK cells to lyse targeted tumor cells. The invention further relates to the use of such multispecific antigen binding proteins in the treatment of cancer.

### BACKGROUND OF THE INVENTION

Immunotherapy of cancer is revolutionizing the treatment of cancers. Cancer immunotherapies are desirable because they are highly specific and can facilitate destruction of a tumor by inducing the recognition and elimination of tumor cells by the patient's own immune system. Recent advances have focused on generating or unleashing tumor antigen-specific T cell responses. They were based on the use of immune checkpoint inhibitors targeting inhibitory pathways, or bispecific T cell engagers and chimeric antigen receptor (CAR) T cells targeting a tumor antigen. Despite these outstanding breakthroughs, the clinical benefit has been limited to a subset of patients and certain tumor types, highlighting the need for alternative strategies.

One such alternative approach is to exploit the antitumor activity of natural killer (NK) cells. NK cells are a component of the innate immune system and make up approximately 15% of circulating lymphocytes. NK cells infiltrate virtually all tissues and were originally characterized by their ability to kill tumor cells effectively without the need for prior sensitization. NK cells provide an efficient immunosurveillance mechanism by which undesired cells such as tumor cells or virally-infected cells can be eliminated. The biological properties of NK cells include the expression of surface antigens including CD16, CD56 and/or CD57, the absence of the α/β or γ/δ TCR complex on the cell surface; the ability to recognize and kill cells that fail to express “self” MHC/HLA antigens by the activation of specific cytolytic enzymes, the ability to kill tumor cells or other diseased cells that express stress ligands for NK activating receptors, and the ability to release protein molecules called cytokines that stimulate or inhibit the immune response. Activated NK cells kill target cells by means similar to cytotoxic T cells, i.e., via cytolytic granules that contain perforin and granzymes as well as via death receptor pathways. Activated NK cells also secrete inflammatory cytokines such as IFN-γ and chemokines that promote the recruitment of other leukocytes to the target tissue. NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD 16 receptors on their surface. The overall sensitivity of NK cells to activation depends on the sum of stimulatory and inhibitory signals.

Strategies based on the recruitment of cytotoxic NK cells are currently being developed. It is expected that treatments based on NK cells are likely to be safer than T-cell treatments, given the absence of graft-versus-host reactions in patients receiving infusions of allogeneic NK cells. Furthermore, unlike chimeric antigen receptor (CAR) T cells, the administration of allogenic CAR-engineered NK cells is not associated with the development of neurotoxicity, cytokine release syndrome (CRS), or graft-versus-host disease, nor does CAR-NK cell infusion increase circulating inflammatory cytokine concentrations above baseline levels. NK cell-based immunotherapies may be less likely to cause these adverse events because the spectrum of cytokines produced by these cells is different from that secreted by T cells. Moreover, due to their inherent ability to discriminate healthy cells from malignant cells, NK cells have a reduced likelihood of killing healthy cells that express the targeted tumor associated antigen (TAA) than T-cells.

More recently, multifunctional antibodies called natural killer cell engagers (NKCEs) have been developed, which simultaneously target tumor-associated antigens (TAAs), and activate receptors on endogenous NK cells. NKCEs are designed to strengthen the interaction between the NK cell and targeted tumor cell and to increase NK cell effector functions towards the tumor cell. A number of NKCEs that are currently in development for clinical application is reviewed by Demaria et al. (Eur. J. Immunol. 2021. 51: 1934-1942).

There remains a need in the art for improved multispecific antigen binding proteins for targeted engagement of NK cells with new and additional functionalities, particularly those that provide therapeutic advantages over existing NKCEs.

### SUMMARY OF THE INVENTION

In a first aspect, the invention relates to a multispecific antigen binding protein comprising: a) a first antigen-binding region that specifically binds a tumor associated antigen (TAA); b) a second antigen-binding region that has affinity for a surface antigen expressed on natural killer (NK) cells; and, c) an NK cell-activating cytokine that is at least one of: i) an interleukin 21 receptor (IL21R) agonist; and, ii) a 4-1BB agonist.

In one embodiment, the first antigen-binding region in the multispecific antigen binding protein comprises at least one immunoglobulin-derived antigen-binding region. The immunoglobulin-derived antigen-binding region can comprise or consist of a Fab or an immunoglobulin single variable domain (ISVD). Preferably, the first antigen-binding region in the multispecific antigen binding protein is a human or humanized antigen-binding region.

In one embodiment, the multispecific antigen binding protein further comprises a third antigen-binding region that specifically binds a TAA or NK cell activating receptor, wherein preferably the third antigen-binding region that specifically binds a TAA can be as defined above for the first antigen-binding region that specifically binds a TAA.

In one embodiment, the multispecific antigen binding protein is a protein wherein the first and third antigen-binding regions bind the same TAA or at least two different TAAs. In one embodiment, the first and third antigen-binding regions are identical.

In one embodiment, the multispecific antigen binding protein is a protein wherein the TAA (that is bound by at least one of the first and third antigen-binding regions) is selected from the group consisting of: Her2 (ErbB2/Neu), Receptor Tyrosine Kinase-like Orphan Receptor 1 (ROR1), Crypto, CD2, CD4, CD20, CD30, CD19, CD38, CD40, CD47, Glycoprotein NMB, CanAg, CD22 (Siglec2), CD33 (Siglec3), CD79, CD123, CD138, CD171, CTLA-4 (CD152), PD1, PSCA, L1-CAM, EpCAM, PSMA (prostate specific membrane antigen), BCMA, TROP2, STEAP1, CD52, CD56, CD80, CD70, E-selectin, EphB2, EPHA4, Melanotransferrin, Mud 6, TMEFF2, Killer Ig-Like Receptor, Killer Ig-Like Receptor 3DL2 (KIR3DL2), B7.1, B7.2, B7-H3, B7-H4, B7-H6, PD-L1, IL-6 receptor, IL-1 accessory protein, MAGE, MART-1/Melan-A, gp100, MICA, MICB, adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), NaPi2b, TYRP1, nectin-4, a UL16-binding protein (ULBP), a RAET1 protein, carcinoembryonic antigen (CEA), CEACAM5, etv6, aml1, prostate specific antigen (PSA), T-cell receptor/CD3-zeta chain, MAGE-A3, a GAGE-tumor antigen, anti-Müllerian hormone Type II receptor, delta-like ligand 3 (DLL3), delta-like ligand 4 (DLL4), DR5, NTRKR1 (EC 2.7.10.1), SLAMF7, TRAILR1, TRAILR2, BAGE, RAGE, LAGE-1, NAG, GnT-V, MUM-1, CDK4, MUC1, MUC1-C, VEGF, VEGFR2, Angiopoietin-2, PDGF, TGF-alpha, EGF, EGF receptor (EGFR/ERBB1), HER-3/ERBB3, HER-4/ERBB4, a heterodimeric receptor comprised of at least one HER subunit, gastrin releasing peptide receptor antigen, cMET, integrin receptors, α5P3 integrins, α5p1 integrins, αIIbp3-integrins, PDGF alpha receptor, PDGF beta receptor, sVE-cadherin, IL-8 receptor, hCG, IL-6 receptor, IL-1 Accessory Protein, CSF1R, α-fetoprotein, mesothelin, Isoform 2 of Claudin-18 (Claudin 18.2), folate receptor alpha (FRα, FOLR1), tissue factor (TF, CD142), P-cadherin, E-cadherin, α-catenin, β-catenin and γ-catenin, Plexin-A1, TNFRSF10B, AXL, EDNRB, OLR1, ADAM12, PLAUR, CCR4, CCR6, p120ctn, PRAME, NY-ESO-1, cdc27, CDCP1, adenomatous polyposis coli protein (APC), fodrin, Connexin 37, Ig-idiotype, p15, gp75, a GM2 ganglioside, a GD2 ganglioside, a human papillomavirus protein, imp-1, P1A, EBV-encoded nuclear antigen (EBNA)-I, brain glycogen phosphorylase, SSX-1, SSX-2 (HOM-MEL-40), SSX-1, SSX-4, SSX-5, SCP-1 CT-7, c-erbB-2, FcRL5/FcRH5, Flt3, muc16, muc17, mmp9, FAP, Lewis-Y, EGFRvIII, GPC3, GPRC5D, gpA33, 5T4, SSTR2, CD73, CD25, CD45, and CD133.

In one embodiment, the multispecific antigen binding protein is a protein wherein at least one of the first and third antigen-binding regions comprises a combination of complementarity-determining regions (CDRs) CDR-H1, CDR-H2, CDR-H3, CDR-L1, CDR-L2 and CDR-L3 selected from the group consisting of: a) the CDR-H1, CDR-H2 and CDR-H3 sequences as comprised in SEQ ID NO: 1, and the CDR-L1, CDR-L2 and CDR-L3 sequences as comprised in SEQ ID NO: 2 (tarstuzumab); b) the CDR-H1 (SEQ ID NO: 152), CDR-H2 (SEQ ID NO: 153) and CDR-H3 (SEQ ID NO: 154) sequences as comprised in SEQ ID NO: 59, and the CDR-L1 (SEQ ID NO: 155), CDR-L2 (SEQ ID NO: 156) and CDR-L3 (SEQ ID NO: 157) sequences as comprised in SEQ ID NO: 60 ((atezolizumab); c) the CDR-H1 (SEQ ID NO: 158), CDR-H2 (SEQ ID NO: 159) and CDR-H3 (SEQ ID NO: 160) sequences as comprised in SEQ ID NO: 9, and the CDR-L1 (SEQ ID NO: 161), CDR-L2 (SEQ ID NO: 162) and CDR-L3 (SEQ ID NO: 163) sequences as comprised in SEQ ID NO: 10 (avelumab); d) the CDR-H1 (SEQ ID NO: 164), CDR-H2 (SEQ ID NO: 165) and CDR-H3 (SEQ ID NO: 166) sequences as comprised in SEQ ID NO: 61, and the CDR-L1 (SEQ ID NO: 167), CDR-L2 (SEQ ID NO: 168) and CDR-L3 (SEQ ID NO: 169) sequences as comprised in SEQ ID NO: 62 (durvalumab); e) the CDR-H1, CDR-H2 and CDR-H3 sequences as comprised in SEQ ID NO: 3, and the CDR-L1, CDR-L2 and CDR-L3 sequences as comprised in SEQ ID NO: 4; f the CDR-H1, CDR-H2 and CDR-H3 sequences as comprised in SEQ ID NO: 5, and the CDR-L1, CDR-L2 and CDR-L3 sequences as comprised in SEQ ID NO: 6; g) the CDR-H1, CDR-H2 and CDR-H3 sequences as comprised in SEQ ID NO: 7, and the CDR-L1, CDR-L2 and CDR-L3 sequences as comprised in SEQ ID NO: 8; h) the CDR-H1, CDR-H2 and CDR-H3 sequences as comprised in SEQ ID NO: 63, and the CDR-L1, CDR-L2 and CDR-L3 sequences as comprised in SEQ ID NO: 64 (cosibelimab); i) the CDR-H1, CDR-H2 and CDR-H3 sequences as comprised in SEQ ID NO: 65, and the CDR-L1, CDR-L2 and CDR-L3 sequences as comprised in SEQ ID NO: 66 (margetuximab); j) the CDR-H1, CDR-H2 and CDR-H3 sequences as comprised in SEQ ID NO: 67, and the CDR-L1, CDR-L2 and CDR-L3 sequences as comprised in SEQ ID NO: 68 (pertuzumab); k) the CDR-H1, CDR-H2 and CDR-H3 sequences as comprised in SEQ ID NO: 69, and the CDR-L1, CDR-L2 and CDR-L3 sequences as comprised in SEQ ID NO: 70 (enoblituzumab); l) the CDR-H1, CDR-H2 and CDR-H3 sequences as comprised in SEQ ID NO: 71, and the CDR-L1, CDR-L2 and CDR-L3 sequences as comprised in SEQ ID NO: 72 (necitumumab); m) the CDR-H1, CDR-H2 and CDR-H3 sequences as comprised in SEQ ID NO: 73, and the CDR-L1, CDR-L2 and CDR-L3 sequences as comprised in SEQ ID NO: 74 (panitumumab); n) the CDR-H1, CDR-H2 and CDR-H3 sequences as comprised in SEQ ID NO: 75, and the CDR-L1, CDR-L2 and CDR-L3 sequences as comprised in SEQ ID NO: 76 (amivantamab EGFR-binding); o) the CDR-H1, CDR-H2 and CDR-H3 sequences as comprised in SEQ ID NO: 77, and the CDR-L1, CDR-L2 and CDR-L3 sequences as comprised in SEQ ID NO: 78 (amivantamab cMet-binding); p) the CDR-H1, CDR-H2 and CDR-H3 sequences as comprised in SEQ ID NO: 79, and the CDR-L1, CDR-L2 and CDR-L3 sequences as comprised in SEQ ID NO: 80 (zolbetuximab); q) the CDR-H1, CDR-H2 and CDR-H3 sequences as comprised in SEQ ID NO: 81, and the CDR-L1, CDR-L2 and CDR-L3 sequences as comprised in SEQ ID NO: 82 (dinutuximab); r) the CDR-H1, CDR-H2 and CDR-H3 sequences as comprised in SEQ ID NO: 83, and the CDR-L1, CDR-L2 and CDR-L3 sequences as comprised in SEQ ID NO: 84 (naxitamab); s) the CDR-H1, CDR-H2 and CDR-H3 sequences as comprised in SEQ ID NO: 85, and the CDR-L1, CDR-L2 and CDR-L3 sequences as comprised in SEQ ID NO: 86 (enfortumab); t) the CDR-H1, CDR-H2 and CDR-H3 sequences as comprised in SEQ ID NO: 87, and the CDR-L1, CDR-L2 and CDR-L3 sequences as comprised in SEQ ID NO: 88 (farletuzumab); u) the CDR-H1, CDR-H2 and CDR-H3 sequences as comprised in SEQ ID NO: 89, and the CDR-L1, CDR-L2 and CDR-L3 sequences as comprised in SEQ ID NO: 90 (tisotumab); v) the CDR-H1, CDR-H2 and CDR-H3 sequences as comprised in SEQ ID NO: 91, and the CDR-L1, CDR-L2 and CDR-L3 sequences as comprised in SEQ ID NO: 92 (mirvetuximab); w) the CDR-H1, CDR-H2 and CDR-H3 sequences as comprised in SEQ ID NO: 93, and the CDR-L1, CDR-L2 and CDR-L3 sequences as comprised in SEQ ID NO: 94 (sacituzumab); x) the CDR-H1, CDR-H2 and CDR-H3 sequences as comprised in SEQ ID NO: 95, and the CDR-L1, CDR-L2 and CDR-L3 sequences as comprised in SEQ ID NO: 96 (vobramitamab); y) the CDR-H1, CDR-H2 and CDR-H3 sequences as comprised in SEQ ID NO: 97, and the CDR-L1, CDR-L2 and CDR-L3 sequences as comprised in SEQ ID NO: 98 (Onartuzumab); z) the CDR-H1, CDR-H2 and CDR-H3 sequences as comprised in SEQ ID NO: 144, and the CDR-L1, CDR-L2 and CDR-L3 sequences as comprised in SEQ ID NO: 145 (sibrotuzumab) aa) the CDR-H1, CDR-H2 and CDR-H3 sequences as comprised in SEQ ID NO: 100, and the CDR-L1, CDR-L2 and CDR-L3 sequences as comprised in SEQ ID NO: 101 (olaratumab); and, ab) the CDR-H1, CDR-H2 and CDR-H3 sequences as comprised in SEQ ID NO: 102, and the CDR-L1, CDR-L2 and CDR-L3 sequences as comprised in SEQ ID NO: 103 (rovalpituzumab). Preferably, the multispecific antigen binding protein is a protein wherein at least one of the first and third antigen-binding regions comprises a combination of variable light (V_{L}) and variable heavy (V_{H}) domains selected from the group consisting of: a) the V_{H} sequence as comprised in SEQ ID NO: 1 and the V_{L} sequence as comprised in SEQ ID NO: 2; b) the VH sequence as comprised in SEQ ID NO: 3 and the V_{L} sequence as comprised in SEQ ID NO: 4; c) the V_{H} sequence as comprised in SEQ ID NO: 5 and the V_{L} sequence as comprised in SEQ ID NO: 6; d) the V_{H} sequence as comprised in SEQ ID NO: 7 and the V_{L} sequence as comprised in SEQ ID NO: 8; and, e) the V_{H} sequence as comprised in SEQ ID NO: 9 and the V_{L} sequence as comprised in SEQ ID NO: 10.

In one embodiment, the multispecific antigen binding protein is a protein wherein the second antigen-binding region comprises or consists of i) an immunoglobulin Fc region or ii) an antigen-binding region that specifically binds a surface antigen expressed on NK cells, wherein preferably, the surface antigen expressed on NK cells is an NK cell activating receptor. Preferably, the Fc region is a dimeric Fc region. In one embodiment, the Fc region is an Fc region that binds to CD16A.

In one embodiment, the Fc region is an Fc region that is modified to reduce or enhance affinity for CD16A, relative to a corresponding wild-type Fc region. In one embodiment, the Fc region is an Fc region that is modified to reduce or enhance NK cell activation through CD16A binding, relative to a corresponding wild-type Fc region.

In one embodiment, the multispecific antigen binding protein is a protein wherein the second antigen-binding region comprises or consists of an antigen-binding region that specifically binds the surface antigen expressed on NK cells, specifically binds an NK cell activating receptor selected from the group consisting of: NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, CRACC, 4-1BB, OX40, CRTAM, CD27, PSGL1, CD96, CD100, CEACAM1, CD59, PD-L1, Tim3 and NTB-A. In one embodiment, the second antigen-binding region activates the NK cell activating receptor.

In one embodiment, the multispecific antigen binding protein is a protein wherein the third antigen-binding region comprises or consists of an antigen-binding region that specifically binds an NK cell activating receptor selected from the group consisting of: NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, CRACC, 4-1BB, OX40, CRTAM, CD27, PSGL1, CD96, CD100, CEACAM1, CD59, PD-L1, Tim3 and NTB-A. In one embodiment, the third antigen-binding region activates the NK cell activating receptor.

In one embodiment, the multispecific antigen binding protein is a protein wherein the IL21R agonist comprises or consist of an IL21 polypeptide or an agonistic antigen-binding region that specifically binds IL21R. In one embodiment, a multispecific antigen binding protein as described herein comprises an IL21R agonist that is an IL21 polypeptide comprising an amino acid sequence with at least 50, 55, 60, 65, 70, 75, 80, 85, 90, 95, 96, 97, 98, 99, or 100% sequence identity to SEQ ID NO: 38, and preferably having an IL21R agonist activity as defined herein, and/or preferably having an affinity for the IL21R as defined herein. In one embodiment, the IL21 polypeptide is an IL21 mutein that is modified to reduce or enhance affinity for IL21R, relative to a corresponding wild type IL21 polypeptide. For example, an IL21 mutein that has reduced affinity for IL21R, relative to a corresponding wild type IL21 polypeptide, can be an IL21 mutein that has a mutation in one or more amino acids selected from the group consisting of, I16, I66, I18, K72, K73, K75, K77, L13, P78, Q12, Q19, R5, R65, R76, R9, S70, S80, V69 and Y23. In one embodiment, the multispecific antigen binding protein is a multispecific antigen binding protein that has an IL21R agonist-valency that is higher than one.

In one embodiment, the multispecific antigen binding protein is a protein wherein the 4-1 BB agonist comprises or consists of at least one 4-1 BB ligand (4-1 BBL) extracellular domain (ECD) or at least one agonistic antigen-binding region that specifically binds 4-1BB. In one embodiment, a multispecific antigen binding protein as described herein comprises a 4-1 BB agonist comprising at least one 4-1 BBL ECD comprising an amino acid sequence with at least 50, 55, 60, 65, 70, 75, 80, 85, 90, 95, 96, 97, 98, 99, or 100% sequence identity to SEQ ID NO: 37, and preferably having an 4-1 BB agonist activity as defined herein, and/or preferably having an affinity for 4-1 BB as defined herein. In one embodiment, the 4-1 BBL ECD is a mutein that is modified to reduce, enhance affinity, improve stability or improve expression for 4-1BB, relative to a corresponding wild type 4-1BBL ECD. In one embodiment, the 4-1 BB agonist comprises or consists of a fusion protein comprising three 4-1 BBL ECD monomers fused together in a single polypeptide chain, and wherein, optionally, the three 4-1BBL ECD monomers are connected by polypeptide linkers. In one embodiment, the multispecific antigen binding protein is a multispecific antigen binding protein that has a 4-1BB agonist-valency that is higher than one.

In one embodiment, the multispecific antigen binding protein is a multispecific antigen binding protein comprising at least one IL21R agonist and at least one 4-1BB agonist.

In one embodiment, the multispecific antigen binding protein is a multispecific antigen binding protein further comprising an NK cell-activating cytokine selected from the group consisting of: an IL15 receptor agonist, an IL2 receptor agonist, a type I interferon (IFN-1) agonist, an IL12 receptor agonist and an IL18 receptor agonist

In one embodiment, the multispecific antigen binding protein is a multispecific antigen binding protein wherein at least one of the first and third antigen-binding regions that specifically binds a TAA is conjugated to the second antigen-binding region that has affinity for a surface antigen expressed on NK cells. In one embodiment, at least one polypeptide chain in the at least one of the first and third antigen-binding regions forms a single polypeptide chain with at least one polypeptide chain of the second antigen-binding region. Preferably, the single polypeptide chain comprises in an N- to C-terminal order: i) at least one polypeptide chain in the at least one of the first and third antigen-binding region; ii) optionally a flexible linker; and iii) the second antigen-binding region.

Preferably, the second antigen-binding region is a dimeric Fc region, wherein each of the two polypeptide chains of the dimeric Fc region is linked to a CH1 domain, each of which CH1 domains is linked to an immunoglobulin-derived antigen-binding region that specifically binds a TAA, whereby the two immunoglobulin-derived antigen-binding regions can bind the same TAA, or whereby the two immunoglobulin-derived antigen-binding region can each bind a different TAA. In a preferred embodiment, the multispecific antigen binding protein is a protein comprising a dimeric Fc region, wherein each of the two Fc polypeptide chains is operably linked to a Fab that specifically binds a TAA.

In one embodiment, the multispecific antigen binding protein is a multispecific antigen binding protein wherein at least one of the NK cell-activating cytokines, is conjugated to the at least one antigen-binding region that specifically binds a TAA, or to the second antigen-binding region. In one embodiment, at least one of the NK cell-activating cytokines forms a single polypeptide chain with at least one of: i) at least one polypeptide chain in at least one of the first and third antigen-binding regions; and, ii) at least one polypeptide chain in the second antigen-binding region; wherein optionally, a flexible linker is present between the agonist and the at least one polypeptide chain in the region defined in i) or ii). In one embodiment, at least one of the NK cell-activating cytokines forms a single polypeptide chain with at least one of: i) a light chain in at least one of the two Fabs that specifically bind a TAA; and, ii) at least one of the two Fc chains in the dimeric Fc region; wherein optionally, a flexible linker is present between the agonist and the light chain defined in i) or the Fc chain defined in ii). In one embodiment, at least one of the NK cell-activating cytokines is fused to at least one of: i) the N-terminus of the light chain in at least one of the two Fabs that specifically bind a TAA, optionally through a flexible linker; ii) the C-terminus of the light chain in at least one of the two Fabs that specifically bind a TAA, optionally through a flexible linker; iii) the N-terminus of the heavy chain in at least one of the two Fabs that specifically bind a TAA; and, iv) the C-terminus of the heavy chain in at least one of the two Fc chains in the dimeric immunoglobulin Fc domain, optionally through a flexible linker. In one embodiment, at least one of the NK cell-activating cytokines is present on at least one or on both sides of the immunoglobulin structure.

In one embodiment, the multispecific antigen binding protein is a multispecific antigen binding protein wherein the multispecific antigen binding protein is heterodimeric with respect to at least one of i) the first and third antigen-binding regions; and ii) at least one fused NK cell-activating cytokine, and wherein the dimeric Fc region comprises different first and a second polypeptide chains comprising knob-into-hole modifications promoting association of the first and the second polypeptide chains of the Fc region.

In one embodiment, the multispecific antigen binding protein is a multispecific antigen binding protein wherein the multispecific antigen binding protein has at least one biological activity selected from: a) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are not brought into contact with the multispecific antigen binding protein; and, b) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are brought into contact with a conventional human IgG1 monoclonal antibody that has the same TAA-specific antigen-binding regions as the multispecific antigen binding protein.

In one embodiment, the multispecific antigen binding protein is a multispecific antigen binding protein wherein ex vivo expansion of donor NK cells by co-culturing with the multispecific antigen binding protein as described herein, produces a population of expanded NK cells having one or more features selected from: a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells); b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells; d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and, e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells, and wherein, preferably the NK cells are co-cultured with tumor cells expressing a TAA specifically bound by the multispecific antigen binding protein.

In a second aspect, the invention pertains to a pharmaceutical composition comprising a multispecific antigen binding protein as described herein, and a pharmaceutically acceptable carrier.

In a third aspect, the invention relates to an ex vivo method for expansion of NK cells, the method comprising the step of contacting an NK cell with a multispecific antigen binding protein as described herein, or with the pharmaceutical composition comprising the protein, wherein preferably the expanded NK cells have one or more features selected from: a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells); b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells; d) the secretion of at least one cytokine of TNF-α, IFN-γ, IL-2 and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells, and wherein, preferably the method comprising the further step of co-culturing the NK cells with tumor cells expressing a TAA specifically bound by the multispecific antigen binding protein.

In one embodiment, the multispecific antigen binding protein causes an increase in Glut1, Glut3, CD71, and/or CD98 expression, mitochondrial mass, glycolytic rate, ratio of glycolytic rate to oxidative phosphorylation rate, metabolic fuel flexibility among glucose, glutamine, and/or fatty acids, whereby preferably, the increase is at least a factor 0.05 higher as compared to the increase achieved with the same effector:target cell ratio, with the same NK cells and target cells that are not brought into contact with the multispecific antigen binding protein; b) the multispecific antigen binding protein causes an increase in at least one NK cell activity selected from Glut1, Glut3, CD71, or CD98 expression, mitochondrial mass, glycolytic rate, ratio of glycolytic rate to oxidative phosphorylation rate, metabolic fuel flexibility among glucose, glutamine, and fatty acids, whereby preferably, the increase is at least a factor 0.05 higher as compared to the increase achieved with the same effector:target cell ratio, with the same NK cells and target cells that are brought into contact with a conventional human IgG1 monoclonal antibody that has the same TAA-specific antigen-binding regions as the multispecific antigen binding protein.

In one embodiment ex vivo expansion of donor NK cells by co-culturing with the multispecific antigen binding protein as described herein, produces a population of expanded NK cells having one or more features selected from: a) mitochondrial mass or the expression of at least one nutrient transporter of Glut1, Glut3, CD71, or CD98 that is at least 0.001, 0.002, 0.005, 0.01, 0.02, 0.05, 0.1, 0.2, 0.5, 1.0, 2.0, 5.0, 10.0, or 20 fold the mitochondrial mass or expression of the nutrient transporter on expanded NK cells obtained by ex vivo expansion by co-culturing with irradiated K562 feeder cells modified to express membrane bound IL-21 (mbIL-21) and 4-1BB ligand (FC21 feeder cells); b) the glycolytic rate is at least 0.01, 0.02, 0.05, 0.1, 0.2, 0.5, 1.0, 2.0, 5.0, 10.0, or 20.0 fold the glycolytic rate of expanded NK cells obtained by ex vivo expansion by co-culturing with irradiated K562 feeder cells modified to express membrane bound IL-21 (mbIL-21) and 4-1BB ligand (FC21 feeder cells); c) the ratio of glycolytic rate to oxidative phosphorylation (OxPhos) rate is at least 0.01, 0.02, 0.05, 0.1, 0.2, 0.5, 1.0, 2.0, 5.0, 10.0, or 20.0 fold the ratio of glycolytic rate to OxPhos rate of expanded NK cells obtained by ex vivo expansion by co-culturing with irradiated K562 feeder cells modified to express membrane bound IL-21 (mbIL-21) and 4-1BB ligand (FC21 feeder cells); d) metabolic fuel flexibility among glucose, glutamine, and fatty acids is at least 0.01, 0.02, 0.05, 0.1, 0.2, 0.5, 1.0, 2.0, 5.0, or 10.0 fold the metabolic fuel flexibility among glucose, glutamine, and fatty acids of expanded NK cells obtained by ex vivo expansion by co-culturing with irradiated K562 feeder cells modified to express membrane bound IL-21 (mbIL-21) and 4-1BB ligand (FC21 feeder cells).

In a fourth aspect, the invention pertains to a multispecific antigen binding protein as described herein, pharmaceutical composition comprising the protein, or an ex vivo expanded NK cell obtained in the above method, optionally in combination with the multispecific antigen binding protein, for use as a medicament.

In a fifth aspect, the invention relates to a multispecific antigen binding protein as described herein, a pharmaceutical composition comprising the protein, or an ex vivo expanded NK cell obtained in the above method, optionally in combination with the multispecific antigen binding protein, for use in the treatment of a cancer, preferably a cancer comprising tumor cells expressing the TAA. In one embodiment, the invention relates to a multispecific antigen binding protein as described herein, or a pharmaceutical composition comprising the protein for use in the treatment of a cancer, preferably a cancer comprising tumor cells expressing the TAA, wherein the multispecific antigen binding protein or the composition is used in combination with an adoptive transfer of immune cells, wherein preferably the immune cells are selected from T cells and NK cells. In one embodiment, the invention relates to the multispecific antigen binding protein, the composition comprising the protein, or the ex vivo expanded NK cell, optionally in combination with the multispecific antigen binding protein, for the above uses, wherein at least one of: a) the multispecific antigen binding protein and/or the ex vivo expanded NK cell is administered as a neoadjuvant therapy before a primary therapy comprising at least one of surgery and radiation therapy of the cancer; and, b) the multispecific antigen binding protein and/or the ex vivo expanded NK cell is administered as an adjuvant therapy after a primary therapy comprising at least one of surgery and radiation therapy of the cancer.

In a sixth aspect, the invention relates to method for enhancing anti-tumor activity of an NK cell in a subject, the method comprising the step of administering to the subject a multispecific antigen binding protein as described herein, pharmaceutical composition comprising the protein, an ex vivo expanded NK cell obtained in the above method, optionally in combination with the multispecific antigen binding protein, or a combination of the multispecific antigen binding protein and an immune cell selected from T cells and NK cells. In one embodiment of the method, the subject has cancer, preferably a cancer comprising tumor cells expressing the TAA.

In a seventh aspect, the invention relates to a nucleic acid molecule comprising one or more nucleotide sequences encoding a polypeptide chain of a multispecific antigen binding protein as described herein. Preferably, the nucleic acid molecule is a nucleic acid molecule wherein the one or more nucleotide sequences are operably linked to regulatory sequences for expression of the one or more polypeptide chains in a host cell.

In an eighth aspect, the invention relates to a host cell comprising a nucleic acid molecule as defined above.

In a ninth aspect, the invention relates to a method for producing a multispecific antigen binding protein as described herein, the method comprising culturing a host cell as defined above, such that one or more nucleotide sequences are expressed, and the multispecific antigen binding protein is produced. Preferably, the method further comprises the steps of: recovery of the multispecific antigen binding protein, and, optionally, formulation of the multispecific antigen binding protein with a pharmaceutically acceptable carrier.

### DESCRIPTION OF THE INVENTION

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein.

Although any methods and materials similar or equivalent to those described herein can be used in the practice for testing of the present invention, the preferred materials and methods are described herein.

“A,” “an,” and “the”: these singular form terms include plural referents unless the content clearly dictates otherwise. The indefinite article “a” or “an” thus usually means “at least one”. Thus, for example, reference to “a cell” includes a combination of two or more cells, and the like.

“About” and “approximately”: these terms, when referring to a measurable value such as an amount, a temporal duration, and the like, is meant to encompass variations of ±20% or ±10%, more preferably ±5%, even more preferably 1%, and still more preferably ±0.1% from the specified value, as such variations are appropriate to perform the disclosed methods. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: The term “and/or” refers to a situation wherein one or more of the stated cases may occur, alone or in combination with at least one of the stated cases, up to with all of the stated cases.

“Comprising”: this term is construed as being inclusive and open ended, and not exclusive. Specifically, the term and variations thereof mean the specified features, steps or components are included. These terms are not to be interpreted to exclude the presence of other features, steps or components.

“Exemplary”: this term means “serving as an example, instance, or illustration,” and should not be construed as excluding other configurations disclosed herein.

As used herein “cancer” and “cancerous”, refer to or describe the physiological condition in mammals that is typically characterized by unregulated cell growth. Cancer is also referred to as malignant neoplasm.

As used herein, “in combination with” is intended to refer to all forms of administration that provide a first drug together with a further (second, third) drug. The drugs may be administered simultaneous, separate or sequential and in any order. Drugs administered in combination have biological activity in the subject to which the drugs are delivered.

As used herein “simultaneous” administration refers to administration of more than one drug at the same time, but not necessarily via the same route of administration or in the form of one combined formulation. For example, one drug may be provided orally whereas the other drug may be provided intravenously during a patient's visit to a hospital. Separate includes the administration of the drugs in separate form and/or at separate moments in time, but again, not necessarily via the same route of administration. Sequentially indicates that the administration of a first drug is followed, immediately or in time, by the administration of the second drug.

A used herein “compositions”, “products” or “combinations” useful in the methods of the present disclosure include those suitable for various routes of administration, including, but not limited to, intravenous, subcutaneous, intradermal, subdermal, intranodal, intratumoral, intramuscular, intraperitoneal, oral, nasal, topical (including buccal and sublingual), rectal, vaginal, aerosol and/or parenteral or mucosal application. The compositions, formulations, and products according to the disclosure invention normally comprise the drugs (alone or in combination) and one or more suitable pharmaceutically acceptable excipients.

As used herein, “an effective amount” is meant the amount of an agent required to ameliorate the symptoms of a disease relative to an untreated patient. The effective amount of active agent(s) used to practice the present invention for therapeutic treatment of a cancer varies depending upon the manner of administration, the age, body weight, and general health of the subject. Ultimately, the attending physician or veterinarian will decide the appropriate amount and dosage regimen. Such amount is referred to as an “effective” amount. Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

As used herein, the phrase “NK cells” refers to a sub-population of lymphocytes that is involved in innate immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to recognize and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (2000, edited by Campbell KS and Colonna M). Humana Press, pp. 219-238).

“Sequence identity” is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The terms “sequence identity” or “sequence similarity” means that two (poly)peptide or two nucleotide sequences, when optimally aligned, preferably over the entire length (of at least the shortest sequence in the comparison) and maximizing the number of matches and minimizes the number of gaps such as by the programs ClustalW (1.83), GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). A preferred multiple alignment program for aligning protein sequences of the invention is ClustalW (1.83) using a blosum matrix and default settings (Gap opening penalty:10; Gap extension penalty: 0.05). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred. Alternatively, percentage similarity or identity may be determined by searching against public databases, using algorithms such as FASTA, BLAST, etc.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes.

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues.

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P |
| | and T |
| Flexible residues | Q, T, K, S, G, P, D, E, and R |

The term “agent” refers generally to any entity which is normally not present or not present at the levels being administered to a cell, tissue or subject. An agent can be a compound or a composition. An agent can e.g. be selected from the group consisting of: polynucleotides, polypeptides, small molecules, (multispecific) antigen binding proteins, such as antibodies and functional fragments thereof.

The term “antigen-binding domain” or “antigen-binding region” refers to the portion of an antigen-binding protein that is capable of specifically binding to an antigen or epitope. In one embodiment, the antigen-binding region is an immunoglobulin-derived antigen-binding region, e.g. comprising both an antibody light chain variable region (V_{L}) and an antibody heavy chain variable region (V_{H}). Examples of such antigen-binding regions include single-chain Fv (scFv), single-chain antibody, Fv, single-chain Fv2 (scFv2), Fab, and Fab′. In one embodiment, the antigen-binding region is an immunoglobulin-derived antigen-binding region from a single domain antibody consisting only of heavy chains and devoid of light chains as are known e.g. from camelids, wherein the antigen-binding site is present on, and formed by, the single variable domain (also referred to as an “immunoglobulin single variable domain” or “ISVD”). Examples of such ISVDs include the single variable domains of camelid heavy chain antibodies (V_{H}Hs), also referred to as nanobodies, domain antibodies (dAbs), and single domains derived from shark antibodies (IgNAR domains). In other embodiments, an antigen-binding region comprises a non-immunoglobulin-derived domain capable of specifically binding to an antigen or epitope, such as DARPpins; Affilins; anticalins, etc.

The term “antibody” herein is used in the broadest sense and specifically includes full-length monoclonal antibodies, polyclonal antibodies, multispecific antibodies (e.g. bispecific antibodies), and antibody fragments and derivatives, so long as they exhibit the desired biological and/or immunological activity. Various techniques relevant to the production of antibodies are provided in, e.g., Harlow, et al., Antibodies: A Laboratory Manual, Cold Spring Harbor Laboratory Press, Cold Spring Harbor, N.Y., (1988). An antibody can be human and/or humanized. “Humanized” forms of non-human (e.g., rodent) antibodies are chimeric antibodies that contain minimal sequence derived from the non-human antibody.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or m (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” comprises a portion of a full-length antibody, e.g. the antigen-binding or variable regions thereof. Examples of antibody fragments include Fab, Fab′, F(ab)2, F(ab′)2, F(ab)s, Fv (typically the V_{H} and V_{L} domains of a single arm of an antibody), single-chain Fv (scFv), dsFv, Fd fragments (typically the V_{H} and CH1 domain), and dAb (typically a V_{H} domain) fragments; V_{H}, V_{L}, V_{H}H, and V-NAR domains; minibodies, diabodies, triabodies, tetrabodies, and kappa bodies (see, e.g.. III et al., Protein Eng 1997;10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, N.Y., pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Various types of antibody fragments have been described or reviewed in, e.g.. Heiliger and Hudson, Nat Biotechnol 2005; 23, 1126-1136; WO2005/040219, US20050238646 and US20020161201. Antibody fragments can be made by various techniques, including but not limited to proteolytic digestion of an intact antibody as well as production by recombinant host cells (e.g. CHO, E. coli or phage), as described herein.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow and Lane, “Antibodies: A Laboratory Manual,” Cold Spring Harbor Laboratory Press, N.Y. (1988); Hammerling et al., in: “Monoclonal Antibodies and T-Cell Hybridomas,” Elsevier, N.Y. (1981), pp. 563-681 (both of which are incorporated herein by reference in their entireties).

The term “monospecific” antibody as used herein denotes that the antibody-part of a multispecific antigen binding protein as described herein, has one or more antigen-binding sites each of which bind to the same epitope of the same antigen. The term “bispecific” means that the antibody-part of a multispecific antigen binding protein as described herein, has at least two antigen-binding sites that are able to specifically bind to at least two distinct antigenic determinants. Typically, a bispecific antigen binding molecule comprises two antigen-binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” or “valency” as used within the current application denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen. Methods for producing and screening for specific antibodies using hybridoma technology are routine and well known in the art. In a non-limiting example, mice can be immunized with an antigen of interest or a cell expressing such an antigen. Once an immune response is detected, e.g., antibodies specific for the antigen are detected in the mouse serum, the mouse spleen is harvested and splenocytes isolated. The splenocytes are then fused by well-known techniques to any suitable myeloma cells. Hybridomas are selected and cloned by limiting dilution. The hybridoma clones are then assayed by methods known in the art for cells that secrete antibodies capable of binding the antigen. Ascites fluid, which generally contains high levels of antibodies, can be generated by inoculating mice intraperitoneally with positive hybridoma clones.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The sequences of the framework regions of different light or heavy chains are relatively conserved within a species. The framework region of an antibody, which is the combined framework regions of the constituent light and heavy chains, serves to position and align the CDRs in three dimensional space.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991, Sequences of Proteins of Immunological Interest, 5th Ed. Public Health Service, National Institutes of Health, Bethesda, MD, USA) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, an FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “framework” or “FR” residues as used herein refers to the region of an antibody variable domain exclusive of those regions defined as CDRs. Each antibody variable domain framework can be further subdivided into the contiguous regions separated by the CDRs (FR1, FR2, FR3 and FR4).

The term “constant region” as defined herein refers to an antibody-derived constant region that is encoded by one of the light or heavy chain immunoglobulin constant region genes. By “constant light chain” or “light chain constant region” as used herein is meant the region of an antibody encoded by the kappa (Cκ) or lambda (Cλ) light chains. The constant light chain typically comprises a single domain, and as defined herein refers to positions 108-214 of Cκ or Cλ, wherein numbering is according to the EU index (Kabat et al., 1991, supra).

The term “constant heavy chain” or “heavy chain constant region” as used herein refers to the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

Papain digestion of intact antibodies produces two identical antigen-binding fragments, called “Fab” fragments containing each the heavy- and light-chain variable domains and also the constant domain of the light chain and the first constant domain (CH1) of the heavy chain. “Fab” fragments can also be recombinantly produced by methods known in the art. As used herein, Thus, the term “Fab fragment” “or “Fab region” refers to an antibody fragment comprising a light chain fragment comprising a VL domain and a constant domain of a light chain (CL), and a VH domain and a first constant domain (CH1) of a heavy chain. Fab may refer to this region in isolation, or this region in the context of a polypeptide, multispecific antigen binding protein or antigen-binding region, or any other embodiments as outlined herein. Fab′ fragments differ from Fab fragments by the addition of a few residues at the carboxy terminus of the heavy chain CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-combining sites (two Fab fragments) and a part of the Fc region.

The term “single-chain Fv” or “scFv” as used herein refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds.

Springer-Verlag, N.Y., pp. 269-315 (1994).

“Scaffold antigen-binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen-binding protein is selected from the group consisting of CTLA-4 (Evibody), Lipocalins (Anticalin), monobodies, centyrins, kunitz domains, knottins, fynomers, lipocalins, a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a Kunitz type domain of human protease inhibitors, microbodies such as the proteins from the knottin family, peptide aptamers and fibronectin (adnectin).

CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001).

Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size and are derived from lipocalins. For further details see Biochim Biophys Acta 1482: 337-350 (2000), U.S. Pat. No. 7,250,297B1 and US20070224633.

An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 17, 455-462 (2004) and EP1641818A1.

Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007).

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33-residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

A single-domain antibody is an antibody fragment consisting of a single monomeric variable antibody domain. The first single variable domains were derived from the variable domain of the antibody heavy chain from camelids (nanobodies or V_{H}H fragments). Furthermore, the term single variable domain antibody includes an autonomous human heavy chain variable domain (aVH) or V_{NAR} fragments derived from sharks.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to 25 amino acids without affecting the overall fold of the microprotein. For further details of engineered knottin domains, see WO2008098796.

The term “Fv” or “Fv fragment” or “Fv region” as used herein refers to a polypeptide that comprises the V_{H} and V_{L} domains of a single antibody.

The term “Fc” or “Fc region”, as used herein refers to the polypeptide comprising the constant region of an antibody excluding the first constant region immunoglobulin domain. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides herein include but are not limited to antibodies, Fc fusions and Fc fragments. Also, Fc regions according to the invention include variants containing at least one modification that alters (enhances or diminishes) an Fc associated effector function. Also, Fc regions according to the invention include chimeric Fc regions comprising different portions or domains of different Fc regions, e.g., derived from antibodies of different isotype or species. Fc thus refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cy 3 (CH3) and the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference).

Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc region, and the hole modification comprises the amino acid substitutions T366S, L368A and Y407V in the other one of the two subunits of the Fc domain. In a further specific embodiment, the subunit of the Fc region comprising the knob modification additionally comprises the amino acid substitution S354C, and the subunit of the Fc region comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). The numbering is according to EU index of Kabat et al, Sequences of Proteins of Immunological Interest, 5th Ed. Public Health Service, National Institutes of Health, Bethesda, MD, 1991.

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcaRI (CD89). A particular activating Fc receptor is human FcγRIIIa (see UniProt accession no. P08637, version 141), also referred to as CD16 or CD16A. In humans, CD16 consists of two isoforms, CD16A and CD16B, encoded by two highly homologous genes. CD16A is a transmembrane protein expressed by lymphocytes and some monocytes, whereas CD16B is linked to the plasma membrane via a GPI anchor and primarily expressed by neutrophils. Therefore, when reference is made herein to CD16 in the context of expression on NK cells herein, usually CD16A is meant unless otherwise indicated.

By “variable region” as used herein is meant the region of an antibody that comprises one or more Ig domains substantially encoded by any of the V_{L} (including Vκ and Vλ) and/or V_{H} genes that make up the light chain (including κ and λ) and heavy chain immunoglobulin genetic loci respectively. A light or heavy chain variable region (V_{L} or V_{H}) comprise four conserved framework regions (FRs) and three hypervariable regions (HVRs). See, e.g., Kindt et al., Kuby Immunology, 6th ed., W. H. Freeman and Co., page 91 (2007). A single V_{H} or V_{L} domain may be sufficient to confer antigen-binding specificity.

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the V_{H} (H1, H2, H3), and three in the V_{L} (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (HI), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) Hypervariable regions (HVRs) are also referred to as complementarity determining regions (CDRs), and these terms are used herein interchangeably in reference to portions of the variable region that form the antigen-binding regions. This particular region has been described by Kabat et al., U.S. Dept. of Health and Human Services, “Sequences of Proteins of Immunological Interest” (1983) and by Chothia et al., J. Mol. Biol. 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table A as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE A
CDR defintions¹

| CDR | Kabat | Chotia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table A is according to the numbering conventions set forth by Kabat et al. (see below).
²“AbM” with a lowercase “b” as used in Table A refers to the CDRs as defined by Oxford Molecular's “AbM” antibody modeling software.

Kabat et aL. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDRL2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

As used herein, the term “affinity matured” in the context of antigen binding molecules (e.g., antibodies) refers to an antigen-binding molecule that is derived from a reference antigen-binding molecule, e.g., by mutation, binds to the same antigen, preferably binds to the same epitope, as the reference antibody; and has a higher affinity for the antigen than that of the reference antigen-binding molecule. Affinity maturation generally involves modification of one or more amino acid residues in one or more CDRs of the antigen-binding molecule. Typically, the affinity matured antigen-binding molecule binds to the same epitope as the initial reference antigen-binding molecule.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG1, IgG2, IgG3, IgG4, IgA1, and IgA2. The heavy chain constant domains that correspond to the different classes of immunoglobulins are called α, δ, ε, γ, and m respectively.

A “blocking” antibody or an “antagonist” antibody is one which inhibits or reduces biological activity of the antigen it binds. Preferred blocking antibodies or antagonist antibodies substantially or completely inhibit the biological activity of the antigen. An “agonist antibody”, as used herein, is an antibody which mimics at least one of the functional activities of a polypeptide of interest.

The term “specifically binds” refers to the number of different types of antigens or antigenic determinants to which a particular antigen-binding region or antigen-binding protein can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein. Alternatively, the affinity can also be expressed as the affinity constant (K_{A}), which is 1/K_{D}. Affinity can be determined in a manner known per se, depending on the specific combination of antigen-binding protein and antigen of interest. Avidity is herein understood to refer to the strength of binding of a target molecule with multiple binding sites by a larger complex of binding agents, i.e. the strength of binding of multivalent binding. Avidity is related to both the affinity between an antigenic determinant and its antigen-binding site on the antigen-binding protein and the valency, i.e. the number of binding sites present on the antigen-binding protein. Affinity, on the other hand refers to simple monovalent receptor ligand systems.

Typically, an antigen-binding region of a multispecific antigen binding protein of the invention thereof will specifically bind its target molecule (antigen) with a dissociation constant (K_{D}) of about 10⁻⁶ to 10⁻¹² M or less, and preferably 10⁻⁸ to 10⁻¹² M or less, and/or with a binding affinity of at least 10⁻⁶ M or 10⁻⁷ M, preferably at least 10⁻⁸ M, more preferably at least 10⁻⁹ M, such as at least 10⁻¹⁰ 10⁻¹¹, 10⁻¹² M or less. Any K_{D} value greater than 10⁻⁴ M (i.e. less than 100 μM) is generally considered to indicate non-specific binding. Thus, an antigen-binding region that “specifically binds” an antigen, is an antigen-binding domain that binds the antigen with a K_{D} value of no more than 10⁻⁴ M, as may be determined as herein described below. Preferably, an antigen-binding region of a multispecific antigen binding protein of the invention will specifically bind to the target molecule with an affinity less than 800, 400, 200, 100 50, 10 or 5 nM, more preferably less than 1 nM, such as less than 500, 200, 100, 50, 10 or 5 pM. A variety of methods of measuring binding affinity are known in the art, any of which can be used for purposes of the present invention (see e.g. Harlow, et al., Antibodies: A Laboratory Manual, Cold Spring Harbor Laboratory Press, Cold Spring Harbor, N.Y., 1988), Coligan et al., eds.. Current Protocols in Immunology, Greene Publishing Assoc, and Wiley Interscience, N.Y., (1992, 1993), and Muller, Meth. Enzymol. 92:589-601 (1983)). Specific illustrative embodiments are described in the following.

A “K_{D}” or “K_{D} value” can be measured by using an ELISA as described in the Examples herein or by using surface plasmon resonance assays using a BIAcore™-2000 or a BIAcore™-3000 (BIAcore, Inc., Piscataway, NJ) In an exemplary method, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μg/ml (˜0.2 μM) before injection at a flow rate of 5 μl/minute to achieve approximately 10 response units (RU) of coupled protein. Following the injection of antigen, 1M ethanolamine is injected to block unreacted groups. For kinetics measurements, two-fold serial dilutions of the antibody or Fab (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (K_{D}) is calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-Aminco spectrophotometer (ThermoSpectronic) with a stir red cuvette.

The term “humanized antibody” or “humanized immunoglobulin” refers to an immunoglobulin comprising a human framework, at least one and preferably all complementarity determining regions (CDRs) from a non-human antibody, and in which any constant region present is substantially identical to a human immunoglobulin constant region, i.e., at least about 85%, at least 90%, and at least 95% identical. Hence, all parts of a humanized immunoglobulin, except possibly the CDRs, are substantially identical to corresponding parts of one or more native human immunoglobulin sequences. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos. 5,530,101; 5,585,089; 5,693,761; 5,693,762; 6,180,370 (each of which is incorporated by reference in its entirety). Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489 498 (1991); Studnicka et al., Prot. Eng. 7:805 814 (1994); Roguska et al., Proc. Natl. Acad. Sci. 91:969 973 (1994), and chain shuffling (U.S. Pat. No. 5,565,332), all of which are hereby incorporated by reference in their entireties.

One class of antigen-binding regions for use in the invention comprises immunoglobulin single variable domains (ISVDs) with an amino acid sequence that corresponds to the amino acid sequence of a naturally occurring single variable domain, but that has been “humanized”, i.e. by replacing one or more amino acid residues in the amino acid sequence of said naturally occurring single variable domain sequence by one or more of the amino acid residues that occur at the corresponding position(s) in a V_{H} domain from a conventional 4-chain antibody from a human being. This can be performed in a manner known per se, which will be clear to the skilled person, for example on the basis of the prior art on humanization including e.g. Jones et al. (Nature 321:522-525, 1986); Riechmann et al., (Nature 332:323-329, 1988); Presta (Curr. Op. Struct. Biol. 2:593-596, 1992), Vaswani and Hamilton (Ann. Allergy, Asthma and Immunol., 1:105-115 1998); Harris (Biochem. Soc. Transactions, 23:1035-1038, 1995); Hurle and Gross (Curr. Op. Biotech., 5:428-433, 1994), and specific prior art relating to humanization of V_{H}Hs such as e.g. Vincke et al. (2009, J. Biol. Chem. 284:3273-3284). Again, it should be noted that such humanized single variable domains of the invention can be obtained in any suitable manner known per se and thus are not strictly limited to polypeptides that have been obtained using a polypeptide that comprises a naturally occurring single variable domain as a starting material.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in V_{H} (or V_{L}): FR1-H1 (L1)-FR2-H2(L2)-FR3-H3(L3)-FR4.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

As an alternative to humanization, human antibodies can be generated. By “human antibody” is meant an antibody containing entirely human light and heavy chains as well as constant regions, produced by any of the known standard methods. For example, transgenic animals (e.g., mice) are available that are capable, upon immunization, of producing a full repertoire of human antibodies in the absence of endogenous immunoglobulin production. For example, it has been described that the homozygous deletion of the antibody heavy-chain joining region PH gene in chimeric and germ-line mutant mice results in the complete inhibition of endogenous antibody production. Transfer of the human germ-line immunoglobulin gene array in such germ line mutant mice will result in the production of human antibodies after immunization. See, e.g., Jakobovits et al., Proc. Nat. Acad. Sci. USA, 90:255 1 (1993); Jakobovits et al., Nature, 362:255-258 (1993). Alternatively, phage display technology (McCafferty et al., Nature 348:552-553 (1990)) can be used to produce human antibodies and antibody fragments in vitro, from immunoglobulin variable (V) domain gene repertoires from donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-57 1 (1993). Human antibodies may also be generated by in vitro activated B cells or SCID mice with its immune system reconstituted with human cells. Once a human antibody is obtained, its coding DNA sequences can be isolated, cloned and introduced into an appropriate expression system i.e. a cell line, preferably from a mammal, which subsequently express and liberate it into a culture media from which the antibody can be isolated.

The term “tumor associated antigen” (TAA) as used herein means any antigen including but not limited to a protein, glycoprotein, ganglioside, carbohydrate, lipid that is associated with cancer. Such antigen can be expressed on malignant cells or in the tumor microenvironment such as on tumor-associated blood vessels, extracellular matrix, mesenchymal stroma, or immune infiltrates. Expressly included in the term TAA are homologues of a wild-type TAA that differs therefrom as a result of tumor-specific mutations (which can be patient-specific or shared) and that result in altered amino acid sequences, i.e. so-called neoantigens.

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or expression construct” refer to nucleic acid molecules that are capable of effecting expression of a nucleotide sequence or gene in host cells or host organisms compatible with such expression vectors or constructs. These expression vectors typically include regulatory sequence elements that are operably linked to the nucleotide sequence to be expressed to effect its expression. Such regulatory elements usually at least include suitable transcription regulatory sequences and optionally, 3′ transcription termination signals. Additional elements necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention whereas an expression construct will usually integrate in the host cell's genome for it to be maintained. Techniques for the introduction of nucleic acid into cells are well established in the art and any suitable technique may be employed, in accordance with the particular circumstances. For eukaryotic cells, suitable techniques may include calcium phosphate transfection, DEAE-Dextran, electroporation, liposome-mediated transfection and transduction using retrovirus or other virus, e.g. adenovirus, AAV, lentivirus or vaccinia. For microbial, e.g. bacterial, cells, suitable techniques may include calcium chloride transformation, electroporation and transfection using bacteriophage. The introduced nucleic acid may be on an extra-chromosomal vector within the cell or the nucleic acid may be integrated into the genome of the host cell. Integration may be promoted by inclusion of sequences within the nucleic acid or vector which promote recombination with the genome, in accordance with standard techniques.

The introduction may be followed by expression of the nucleic acid to produce the encoded fusion protein. In some embodiments, host cells (which may include cells actually transformed although more likely the cells will be descendants of the transformed cells) may be cultured in vitro under conditions for expression of the nucleic acid, so that the encoded fusion protein polypeptide is produced, when an inducible promoter is used, expression may require the activation of the inducible promoter.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence.

Operably linked means that the DNA sequences being linked are typically contiguous and, where necessary to join two protein encoding regions, contiguous and in reading frame.

The terms “protein” or “polypeptide” are used interchangeably and refer to molecules consisting of a chain of amino acids, without reference to a specific mode of action, size, 3-dimensional structure or origin.

The term “signal peptide” (sometimes referred to as signal sequence) is a short peptide (usually 16-30 amino acids long) present at the N-terminus of the majority of newly synthesized proteins that are destined towards the secretory pathway. At the end of the signal peptide there is usually a stretch of amino acids that is recognized and cleaved by signal peptidase either during or after completion of translocation (from the cytosol into the secretory pathway, i.e. ER) to generate a free signal peptide and a mature protein. Signal peptides are extremely heterogeneous, and many prokaryotic and eukaryotic signal peptides are functionally interchangeable even between different species however the efficiency of protein secretion may depend on the signal peptide. Suitable signal peptides are generally known in the art e.g. from Kǎll et al. (2004 J. Mol. Biol. 338: 1027-1036) and von Heijne (1985, J Mol Biol. 184 (1): 99-105).

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature.

Heterologous nucleic acids or proteins are not endogenous to the cell into which it is introduced but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

### DETAILED DESCRIPTION OF THE INVENTION

The present invention arises in part from the observation that multispecific antigen binding proteins, which bind to a tumor-associated antigen of interest, and which comprise an NK cell-activating cytokines that triggers at least one of the NK cell's interleukin 21 receptor and 4-1 BB, are capable of inducing hyper-functionality in NK cells, which includes their proliferation, resistance to the tumor microenvironment, their enhanced capability to mediate lysis of the target cell and the prolongation of these capabilities (FIG. 1). Such capabilities can be even further enhanced if the multispecific antigen binding protein comprises a domain that has affinity for a surface antigen expressed on NK cells, such as an Fc domain, allowing the multispecific antigen binding protein to induce a tumor-specific Antibody-dependent cellular cytotoxicity by hyper-functional NK cells.

### A Multispecific Antigen Binding Protein

In a first aspect, the invention pertains to a multispecific antigen binding protein. In one embodiment, the multispecific antigen binding protein comprises at least one first antigen-binding region that specifically binds to a tumor associated antigen (TAA) and the multispecific antigen binding protein comprises an NK cell-activating cytokine. The NK cell-activating cytokine preferably is at least one of i) an interleukin 21 receptor (IL21R) agonist; and, ii) a 4-1 BB agonist. Binding of the multi-specific binding protein to a TAA on a tumor cell allows the NK cell-activating cytokine(s) to activate the NK cell in the vicinity of the tumor cell. In one embodiment, the multispecific antigen binding protein further comprises a second antigen-binding region that has affinity for a surface antigen expressed on NK cells. The second antigen-binding region thus brings the NK cell into proximity of the tumor cell, where the NK cell is activated towards killing of the tumor cell.

Thus, in one embodiment, the multispecific antigen binding protein comprises: a) at least one first antigen-binding region that specifically binds a TAA; b) a second antigen-binding region that has affinity for a surface antigen expressed on NK cells; and, c) an NK cell-activating cytokine that is at least one of: i) an interleukin 21 receptor (IL21R) agonist; and, ii) a 4-1 BB agonist.

### Tumor Associated Antigens

In one embodiment, a multispecific antigen binding protein described herein comprises a first antigen-binding region that specifically binds a TAA. The multispecific antigen binding protein can further comprise another, i.e. third antigen-binding region that also specifically binds a TAA, or that can specifically bind to an NK cell activating receptor. The first and third antigen-binding regions can specifically bind the same TAA or they can each bind a different TAA.

An antigen-binding region as used in a multispecific antigen binding protein described herein can be derived from any of a variety of immunoglobulin or non-immunoglobulin scaffolds, for example affibodies based on the Z-domain of staphylococcal protein A, engineered Kunitz domains, monobodies or adnectins based on the 10th extracellular domain of human fibronectin I11, anticalins derived from lipocalins, DARPins (designed ankyrin repeat domains), Affilins, multimerized LDLR-A module, avimers or cysteine-rich knottin peptides. See, e.g., Gebauer and Skerra (2009) Current Opinion in Chemical Biology 13:245-255, the disclosure of which is incorporated herein by reference.

In a preferred embodiment, an antigen-binding region as used in a multispecific antigen binding protein described herein comprises or consists of an immunoglobulin variable region. Such immunoglobulin variable regions can comprise or consist of variable domains that are commonly derived from antibodies (immunoglobulin chains), e.g. in the form of associated V_{L} and V_{H} domains found on two polypeptide chains, such as present in a Fab. Alternatively, immunoglobulin variable domains can comprise or consist of a single chain antigen-binding domain such as a scFv, a V_{H} domain, a V_{L} domain, or an immunoglobulin single variable domain (ISVD) such as a dAb, a V-NAR domain or a V_{H}H domain. An immunoglobulin variable region to be used in a multispecific antigen binding protein described herein can be a human or humanized immunoglobulin variable region or an immunoglobulin single variable domain as herein defined above.

In one embodiment, the antigen-binding region that specifically binds a TAA is an antigen-binding region derived from immunoglobulin or non-immunoglobulin scaffolds as defined above.

Preferably, the antigen-binding region that specifically binds a TAA comprises or consists of at least one immunoglobulin variable domain. More preferably, the antigen-binding region that specifically binds a TAA comprises or consists of a Fab that specifically binds a TAA or an immunoglobulin single variable domain (ISVD) that specifically binds a TAA. In one embodiment, the antigen-binding region that specifically binds a TAA is an antigen-binding region that binds the TAA with a K_{D} value of no more than 10⁻⁴ M, as may be determined as herein described above.

In one embodiment, the antigen-binding region that specifically binds a TAA comprises or consists of a human or humanized immunoglobulin variable region or immunoglobulin single variable region as herein defined above.

In one embodiment, a multispecific antigen binding protein as described herein comprises two antigen-binding regions that specifically bind a TAA, i.e. a first and a third antigen-binding region. In a multispecific antigen binding protein that comprises two antigen-binding regions that specifically bind a TAA, the two antigen-binding regions can bind one and the same TAA or they can bind at least two different TAAs. In one embodiment of a multispecific antigen binding protein that comprises two antigen-binding regions that specifically bind a TAA, the two antigen-binding regions are identical. Thus, as regards the two antigen-binding regions that specifically bind a TAA, a multispecific antigen binding protein as described herein can be a homodimeric or a heterodimeric antigen binding protein.

As used herein, the term tumor-associated antigen (TAA) refers to an antigen that is differentially expressed by cancer/tumor cells as compared to normal, i.e. non-tumoral cells.

Alternatively, a TAA can be an antigen that is expressed by non-tumoral cells (e.g. immune cells) having a pro-tumoral effect (e.g. an immunosuppressive effect), and can thereby be exploited in order to target cancer cells. A TAA can thus be any antigen that potentially stimulates apparently tumor-specific immune responses. Some of these antigens are encoded, although not necessarily expressed, or expressed at lower levels or less frequently, by normal cells. These antigens can be characterized as those which are normally silent (i.e., not expressed) in normal cells, those that are expressed only at certain stages of differentiation and those that are temporally expressed such as embryonic and fetal antigens. Other TAAs are encoded by mutant cellular genes, such as oncogenes (e.g., activated ras oncogene), suppressor genes (e.g., mutant p53), fusion proteins resulting from internal deletions or chromosomal translocations, including neo-antigens. Still other TAAs antigens can be encoded by viral genes such as those carried on RNA and DNA tumor viruses. Still other TAAs can be expressed on immune cells capable of contributing to or mediating a pro-tumoral effect, e.g. cell that contributes to immune evasion, a monocyte or a macrophage, optionally a suppressor T cell, regulatory T cell, or myeloid-derived suppressor cell.

The TAAs are usually normal cell surface antigens which are either overexpressed or expressed at abnormal times or are expressed by a targeted population of cells. Ideally the target TAA is expressed only on proliferative cells (e.g., tumor cells) or pro-tumoral cells (e.g. immune cells having an immunosuppressive effect), however this is rarely observed in practice. As a result, target antigens are in many cases selected on the basis of differential expression between proliferative/disease tissue and healthy tissue.

Examples of TAAs include: Receptor Tyrosine Kinase-like Orphan Receptor 1 (ROR1), Crypto, CD2, CD4, CD20, CD30, CD19, CD38, CD40, CD47, Glycoprotein NMB, CanAg, Her2 (ErbB2/Neu), a Siglec family member, for example CD22 (Siglec2) or CD33 (Siglec3), CD79, CD123, CD138, CD171, CTLA-4 (CD152), PD1, PSCA, L1-CAM, EpCAM, PSMA (prostate specific membrane antigen), BCMA, TROP2, STEAP1, CD52, CD56, CD80, CD70, E-selectin, EphB2, EPHA4, Melanotransferrin, Mud 6 and TMEFF2. Examples of TAAs also include Immunoglobulin superfamily (IgSF) proteins such as cytokine receptors, Killer-Ig Like Receptor, CD28 family proteins, for example, Killer-Ig Like Receptor 3DL2 (KIR3DL2), B7.1, B7.2, B7-H3, B7-H4, B7-H6, PD-L1, IL-6 receptor. Examples of TAAs further include MAGE, MART-1/Melan-A, gp100, major histocompatibility complex class I-related chain A and B polypeptides (MICA and MICB), adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), NaPi2b, TYRP1, nectins (e.g. nectin-4), major histocompatibility complex class I-related chain A and B polypeptides (MICA and MICB), proteins of the UL16-binding protein (ULBP) family, proteins of the retinoic acid early transcript-1 (RAET1) family, carcinoembryonic antigen (CEA) and its immunogenic epitopes CAP-1 and CAP-2, CEACAM5, etv6, amll, prostate specific antigen (PSA), T-cell receptor/CD3-zeta chain, MAGE-family of tumor antigens, e.g. MAGE-A3, GAGE-family of tumor antigens, anti-Müllerian hormone Type II receptor, delta-like ligand 3 (DLL3), delta-like ligand 4 (DLL4), DR5, ROR1 (also known as Receptor Tyrosine Kinase-Like Orphan Receptor 1 or NTRKR1 (EC 2.7.10.1), SLAMF7, TRAILR1, TRAILR2, BAGE, RAGE, LAGE-1, NAG, GnT-V, MUM-1, CDK4, MUC family, e.g. MUC1 or MUC1-C, VEGF, VEGF receptors, Angiopoietin-2, PDGF, TGF-alpha, EGF, EGF receptor (EGFR/ERBB1), members of the human EGF-like receptor family, e.g., HER-2/neu, HER-3/ERBB3, HER-4/ERBB4 or a heterodimeric receptor comprised of at least one HER subunit, gastrin releasing peptide receptor antigen, cMET, integrin receptors, α5β3 integrins, α5β1 integrins, αIIbβ3-integrins, PDGF beta receptor, sVE-cadherin, IL-8 receptor, hCG, IL-6 receptor, IL-1 accessory Protein, CSF1R (tumor-associated monocytes and macrophages), α-fetoprotein, mesothelin, Isoform 2 of Claudin-18 (Claudin 18.2), folate receptor alpha (FRα, FOLR1), tissue factor (TF, CD142), P-cadherin, E-cadherin, α-catenin, β-catenin and γ-catenin, Plexin-A1, TNFRSF10B, AXL, EDNRB, OLR1, ADAM12, PLAUR, CCR6, p120ctn, PRAME, NY-ESO-1, cdc27, CDCP1, adenomatous polyposis coli protein (APC), fodrin, Connexin 37, Ig-idiotype, p15, gp75, GM2 and GD2 gangliosides, viral products such as human papillomavirus proteins, imp-1, P1A, EBV-encoded nuclear antigen (EBNA)-I, brain glycogen phosphorylase, SSX-1, SSX-2 (HOM-MEL-40), SSX-1, SSX-4, SSX-5, SCP-1 and CT-7, c-erbB-2, FcRL5/FcRH5, Flt3, muc16, muc17, mmp9, FAP, Lewis-Y, EGFRvIII, GPC3, GPRC5D, gpA33, 5T4, SSTR2, CD73, CD25, CD45, and CD133, although this is not intended to be exhaustive.

Thus, in one embodiment, a multispecific antigen binding protein as described herein comprises at least one antigen-binding region that specifically binds to a TAA selected from the group consisting of: Her2 (ErbB2/Neu), Receptor Tyrosine Kinase-like Orphan Receptor 1 (ROR1), Crypto, CD2, CD4, CD20, CD30, CD19, CD38, CD40, CD47, Glycoprotein NMB, CanAg, CD22 (Siglec2), CD33 (Siglec3), CD79, CD123, CD138, CD171, CTLA-4 (CD152), PD1, PSCA, L1-CAM, EpCAM, PSMA (prostate specific membrane antigen), BCMA, TROP2, STEAP1, CD52, CD56, CD80, CD70, E-selectin, EphB2, EPHA4, Melanotransferrin, Mud 6, TMEFF2, Killer Ig-Like Receptor, Killer Ig-Like Receptor 3DL2 (KIR3DL2), B7.1, B7.2, B7-H3, B7-H4, B7-H6, PD-L1, IL-6 receptor, IL1 accessory Protein, MAGE, MART-1/Melan-A, gp100, MICA, MICB, adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), NaPi2b, TYRP1, nectin-4, a UL16-binding protein (ULBP), a RAET1 protein, carcinoembryonic antigen (CEA), CEACAM5, etv6, aml1, prostate specific antigen (PSA), T-cell receptor/CD3-zeta chain, MAGE-A3, a GAGE-tumor antigen, anti-Müllerian hormone Type II receptor, delta-like ligand 3 (DLL3), delta-like ligand 4 (DLL4), DR5, NTRKR1 (EC 2.7.10.1), SLAMF7, TRAILR1, TRAILR2, BAGE, RAGE, LAGE-1, NAG, GnT-V, MUM-1, CDK4, MUC1, MUC1-C, VEGF, VEGFR2, Angiopoietin-2, PDGF, TGF-alpha, EGF, EGF receptor (EGFR/ERBB1), HER-3/ERBB3, HER-4/ERBB4, a heterodimeric receptor comprised of at least one HER subunit, gastrin releasing peptide receptor antigen, cMET, integrin receptors, α5β3 integrins, α5β1 integrins, αIIbβ3-integrins, PDGF alpha receptor, PDGF beta receptor, sVE-cadherin, IL-8 receptor, hCG, IL-6 receptor, CSF1R, α-fetoprotein, mesothelin (MSLN), Isoform 2 of Claudin-18 (Claudin 18.2, CLDN18)), folate receptor alpha (FRα, FOLR1), tissue factor (TF, CD142), P-cadherin, E-cadherin, α-catenin, β-catenin and γ-catenin, Plexin-A1, TNFRSF10B, AXL, EDNRB, OLR1, ADAM12, PLAUR, CCR4, CCR6, p120ctn, PRAME, NY-ESO-1, cdc27, CDCP1, adenomatous polyposis coli protein (APC), fodrin, Connexin 37, Ig-idiotype, p15, gp75, a GM2 ganglioside, a GD2 ganglioside, a human papillomavirus protein, imp-1, P1A, EBV-encoded nuclear antigen (EBNA)-I, brain glycogen phosphorylase, SSX-1, SSX-2 (HOM-MEL-40), SSX-1, SSX-4, SSX-5, SCP-1 CT-7, c-erbB-2, FcRL5/FcRH5, Flt3, muc16, muc17, mmp9, FAP, Lewis-Y, EGFRvIII, GPC3, GPRC5D, gpA33, 5T4, SSTR2, CD73, CD25, CD45, and CD133.

In one embodiment, a multispecific antigen binding protein as described herein comprises at least one antigen-binding region that is obtained/obtainable from a cytotoxic monoclonal antibody against a TAA as is known in the art. In one embodiment, the at least one antigen-binding region at least comprises the six CDR sequences that are obtained/obtainable from a monoclonal antibody against a TAA as is known in the art. In one embodiment, the at least one antigen-binding region at least comprises the variable light (V_{L}) domain and variable heavy (V_{H}) domain sequences that are obtained/obtainable from a monoclonal antibody against a TAA as is known in the art. Examples of such monoclonal antibodies against TAAs include: trastuzumab (to HER2), pertuzumab (to HER2), margetuximab (to HER2), rituximab (to CD20), tositumomab (to CD20), ibritumomab (to CD20), obinutuzumab (to CD20), ofatumumab (to CD20), alemtuzumab (to CD52), blinatumomab (to CD19), inebilizumab (to CD19), tafasitamab (to CD19), daratumumab (to CD38), isatuximab (to CD38), polatuzumab (to CD79b), talacotuzumab (to CD123), dinutuximab (to GD2), naxitamab (to GD2), bevacizumab (to VEGF-A), elotuzumab (to SLAMF7), enfortumab (to nectin-4) sacituzumab (to TROP2), mogamulizumab (to CCR4), ipilimumab (to CTLA-4), tremelimumab (to CTLA-4), durvalumab (to PD-L1), pidilizumab (to PD-1), pembrolizumab (to PD-1), nivolumab (to PD-1), cemiplimab (to PD-1), avelumab (to PD-L1), atezolizumab(to PD-L1), cosibelimab (to PD-L1), cetuximab (to EGFR), necitumumab (to EGFR), panitumumab (to EGFR), amivantamab (bispecific to EGFR and cMet), onartuzumab (monovalent to cMet), olaratumab (to PDGFRα), enoblituzumab (to B7-H3), vobramitamab (to B7-H3), zolbetuximab (to isoform 2 of Claudin-18), mirvetuximab (to folate receptor alpha, FRα), farletuzumab (to folate receptor alpha, FRα), tisotumab (tissue factor, CD142), rovalpituzumab (to DLL3), omburtamab (to B7-H3) and ramucirumab (to VEGFR2).

In one embodiment therefore, a multispecific antigen binding protein as described herein comprises a combination of complementarity-determining regions (CDRs) CDR-H1, CDR-H2, CDR-H3, CDR-L1, CDR-L2 and CDR-L3 selected from the group consisting of: a) the CDR-H1 (SEQ ID NO: 24), CDR-H2 (SEQ ID NO: 25) and CDR-H3 (SEQ ID NO: 26) sequences as comprised in SEQ ID NO: 1, and the CDR-L1 (SEQ ID NO: 27), CDR-L2 (SEQ ID NO: 28) and CDR-L3 (SEQ ID NO: 29) sequences as comprised in SEQ ID NO: 2 (trastuzumab); b) the CDR-H1 (SEQ ID NO: 152), CDR-H2 (SEQ ID NO: 153) and CDR-H3 (SEQ ID NO: 154) sequences as comprised in SEQ ID NO: 59, and the CDR-L1 (SEQ ID NO: 155), CDR-L2 (SEQ ID NO: 156) and CDR-L3 (SEQ ID NO: 157) sequences as comprised in SEQ ID NO: 60 (atezolizumab); c) the CDR-H1 (SEQ ID NO: 158), CDR-H2 (SEQ ID NO: 159) and CDR-H3 (SEQ ID NO: 160) sequences as comprised in SEQ ID NO: 9, and the CDR-L1 (SEQ ID NO: 161), CDR-L2 (SEQ ID NO: 162) and CDR-L3 (SEQ ID NO: 163) sequences as comprised in SEQ ID NO: 10 (avelumab); d) the CDR-H1 (SEQ ID NO: 164), CDR-H2 (SEQ ID NO: 165) and CDR-H3 (SEQ ID NO: 166) sequences as comprised in SEQ ID NO: 61, and the CDR-L1 (SEQ ID NO: 167), CDR-L2 (SEQ ID NO: 168) and CDR-L3 (SEQ ID NO: 169) sequences as comprised in SEQ ID NO: 62 (durvalumab); e) the CDR-H1, CDR-H2 and CDR-H3 sequences as comprised in SEQ ID NO: 3, and the CDR-L1, CDR-L2 and CDR-L3 sequences as comprised in SEQ ID NO: 4 (cetuximab); f) the CDR-H1, CDR-H2 and CDR-H3 sequences as comprised in SEQ ID NO: 5, and the CDR-L1, CDR-L2 and CDR-L3 sequences as comprised in SEQ ID NO: 6 (rituximab); g) the CDR-H1, CDR-H2 and CDR-H3 sequences as comprised in SEQ ID NO: 7, and the CDR-L1, CDR-L2 and CDR-L3 sequences as comprised in SEQ ID NO: 8 (daratumab); h) the CDR-H1, CDR-H2 and CDR-H3 sequences as comprised in SEQ ID NO: 63, and the CDR-L1, CDR-L2 and CDR-L3 sequences as comprised in SEQ ID NO: 64 (cosibelimab); i) the CDR-H1, CDR-H2 and CDR-H3 sequences as comprised in SEQ ID NO: 65, and the CDR-L1, CDR-L2 and CDR-L3 sequences as comprised in SEQ ID NO: 66 (margetuximab); j) the CDR-H1, CDR-H2 and CDR-H3 sequences as comprised in SEQ ID NO: 67, and the CDR-L1, CDR-L2 and CDR-L3 sequences as comprised in SEQ ID NO: 68 (pertuzumab); k) the CDR-H1, CDR-H2 and CDR-H3 sequences as comprised in SEQ ID NO: 69, and the CDR-L1, CDR-L2 and CDR-L3 sequences as comprised in SEQ ID NO: 70 (enoblituzumab); I) the CDR-H1, CDR-H2 and CDR-H3 sequences as comprised in SEQ ID NO: 71, and the CDR-L1, CDR-L2 and CDR-L3 sequences as comprised in SEQ ID NO: 72 (necitumumab); m) the CDR-H1, CDR-H2 and CDR-H3 sequences as comprised in SEQ ID NO: 73, and the CDR-L1, CDR-L2 and CDR-L3 sequences as comprised in SEQ ID NO: 74 (panitumumab); n) the CDR-H1, CDR-H2 and CDR-H3 sequences as comprised in SEQ ID NO: 75, and the CDR-L1, CDR-L2 and CDR-L3 sequences as comprised in SEQ ID NO: 76 (amivantamab EGFR-binding); o) the CDR-H1, CDR-H2 and CDR-H3 sequences as comprised in SEQ ID NO: 77, and the CDR-L1, CDR-L2 and CDR-L3 sequences as comprised in SEQ ID NO: 78 (amivantamab cMet-binding); p) the CDR-H1, CDR-H2 and CDR-H3 sequences as comprised in SEQ ID NO: 79, and the CDR-L1, CDR-L2 and CDR-L3 sequences as comprised in SEQ ID NO: 80 (zolbetuximab); q) the CDR-H1, CDR-H2 and CDR-H3 sequences as comprised in SEQ ID NO: 81, and the CDR-L1, CDR-L2 and CDR-L3 sequences as comprised in SEQ ID NO: 82 (dinutuximab); r) the CDR-H1, CDR-H2 and CDR-H3 sequences as comprised in SEQ ID NO: 83, and the CDR-L1, CDR-L2 and CDR-L3 sequences as comprised in SEQ ID NO: 84 (naxitamab); s) the CDR-H1, CDR-H2 and CDR-H3 sequences as comprised in SEQ ID NO: 85, and the CDR-11, CDR-L2 and CDR-L3 sequences as comprised in SEQ ID NO: 86 (enfortumab); t) the CDR-H1, CDR-H2 and CDR-H3 sequences as comprised in SEQ ID NO: 87, and the CDR-L1, CDR-L2 and CDR-L3 sequences as comprised in SEQ ID NO: 88 (farletuzumab); u) the CDR-H1, CDR-H2 and CDR-H3 sequences as comprised in SEQ ID NO: 89, and the CDR-L1, CDR-L2 and CDR-L3 sequences as comprised in SEQ ID NO: 90 (tisotumab); v) the CDR-H1, CDR-H2 and CDR-H3 sequences as comprised in SEQ ID NO: 91, and the CDR-L1, CDR-L2 and CDR-L3 sequences as comprised in SEQ ID NO: 92 (mirvetuximab); w) the CDR-H1, CDR-H2 and CDR-H3 sequences as comprised in SEQ ID NO: 93, and the CDR-L1, CDR-L2 and CDR-L3 sequences as comprised in SEQ ID NO: 94 (sacituzumab); x) the CDR-H1, CDR-H2 and CDR-H3 sequences as comprised in SEQ ID NO: 95, and the CDR-L1, CDR-L2 and CDR-L3 sequences as comprised in SEQ ID NO: 96 (vobramitamab); y) the CDR-H1, CDR-H2 and CDR-H3 sequences as comprised in SEQ ID NO: 97, and the CDR-L1, CDR-L2 and CDR-L3 sequences as comprised in SEQ ID NO: 98 (Onartuzumab); z) the CDR-H1, CDR-H2 and CDR-H3 sequences as comprised in SEQ ID NO: 144, and the CDR-L1, CDR-L2 and CDR-L3 sequences as comprised in SEQ ID NO: 145 (sibrotuzumab) aa) the CDR-H1, CDR-H2 and CDR-H3 sequences as comprised in SEQ ID NO: 100, and the CDR-L1, CDR-L2 and CDR-L3 sequences as comprised in SEQ ID NO: 101 (olaratumab); and, ab) the CDR-H1, CDR-H2 and CDR-H3 sequences as comprised in SEQ ID NO: 102, and the CDR-L1, CDR-L2 and CDR-L3 sequences as comprised in SEQ ID NO: 103 (rovalpituzumab).

In one embodiment, a multispecific antigen binding protein as described herein comprises a combination of variable heavy (V_{H}) and variable light (V_{L}) domains selected from the group consisting of: a) the V_{H} sequence as comprised in SEQ ID NO: 39 and the V_{L} sequence as comprised in SEQ ID NO: 40 (trastuzumab); b) the V_{H} sequence as comprised in SEQ ID NO: 41 and the V_{L} sequence as comprised in SEQ ID NO: 42 (cetuximab); c) the V_{H} sequence as comprised in SEQ ID NO: 43 and the V_{L} sequence as comprised in SEQ ID NO: 44 (rituximab); d) the V_{H} sequence as comprised in SEQ ID NO: 45 and the V_{L} sequence as comprised in SEQ ID NO: 46 (daratumab); e) the V_{H} sequence as comprised in SEQ ID NO: 47 and the V_{L} sequence as comprised in SEQ ID NO: 48 (avelumab); f the V_{H} sequence as comprised in SEQ ID NO: 104 and the V_{L} sequence as comprised in SEQ ID NO: 105 (atezolizumab); g) the V_{H} sequence as comprised in SEQ ID NO: 106 and the V_{L} sequence as comprised in SEQ ID NO: 107 (durvalumab); h) the V_{H} sequence as comprised in SEQ ID NO: 108 and the V_{L} sequence as comprised in SEQ ID NO: 109 (cosibelimab); i) the V_{H} sequence as comprised in SEQ ID NO: 110 and the V_{L} sequence as comprised in SEQ ID NO: 111 (margetuximab); j) the V_{H} sequence as comprised in SEQ ID NO: 112 and the V_{L} sequence as comprised in SEQ ID NO: 113 (pertuzumab); k) the V_{H} sequence as comprised in SEQ ID NO: 114 and the V_{L} sequence as comprised in SEQ ID NO: 115 (enoblituzumab); I) the V_{H} sequence as comprised in SEQ ID NO: 116 and the V_{L} sequence as comprised in SEQ ID NO: 117 (necitumumab); m) the V_{H} sequence as comprised in SEQ ID NO: 118 and the V_{L} sequence as comprised in SEQ ID NO: 119 (panitumumab); n) the V_{H} sequence as comprised in SEQ ID NO: 120 and the V_{L} sequence as comprised in SEQ ID NO: 121 (amivantamab EGFR-binding); o) the V_{H} sequence as comprised in SEQ ID NO: 122 and the V_{L} sequence as comprised in SEQ ID NO: 123 (amivantamab cMet-binding); p) the V_{H} sequence as comprised in SEQ ID NO: 124 and the V_{L} sequence as comprised in SEQ ID NO: 125 (zolbetuximab); q) the V_{H} sequence as comprised in SEQ ID NO: 126 and the V_{L} sequence as comprised in SEQ ID NO: 127 (dinutuximab); r) the V_{H} sequence as comprised in SEQ ID NO: 128 and the V_{L} sequence as comprised in SEQ ID NO: 129 (naxitamab); s) the V_{H} sequence as comprised in SEQ ID NO: 130 and the V_{L} sequence as comprised in SEQ ID NO: 131 (enfortumab); t) the V_{H} sequence as comprised in SEQ ID NO: 132 and the V_{L} sequence as comprised in SEQ ID NO: 133 (farletuzumab); u) the V_{H} sequence as comprised in SEQ ID NO: 134 and the V_{L} sequence as comprised in SEQ ID NO: 135 (tisotumab); v) the V_{H} sequence as comprised in SEQ ID NO: 136 and the V_{L} sequence as comprised in SEQ ID NO: 137 (mirvetuximab); w) the V_{H} sequence as comprised in SEQ ID NO: 138 and the V_{L} sequence as comprised in SEQ ID NO: 139 (sacituzumab); x) the V_{H} sequence as comprised in SEQ ID NO: 140 and the V_{L} sequence as comprised in SEQ ID NO: 141 (vobramitamab); y) the V_{H} sequence as comprised in SEQ ID NO: 142 and the V_{L} sequence as comprised in SEQ ID NO: 143 (onartuzumab); z) the V_{H} sequence as comprised in SEQ ID NO: 144 and the V_{L} sequence as comprised in SEQ ID NO: 145 (sibrotuzumab); aa) the V_{H} sequence as comprised in SEQ ID NO: 146 and the V_{L} sequence as comprised in SEQ ID NO: 147 (olaratumab); ab) the V_{H} sequence as comprised in SEQ ID NO: 148 and the V_{L} sequence as comprised in SEQ ID NO: 149 (rovalpituzumab); and ac) the V_{H} sequence as comprised in SEQ ID NO: 177 and the V_{L} sequence as comprised in SEQ ID NO: 179 (omburtamab).

In one embodiment, a multispecific antigen binding protein as described herein comprises a combination of heavy and light chains selected from the group consisting of: a) a heavy chain comprising the amino acid sequence of SEQ ID NO: 1 and a light chain comprising the amino acid sequence of SEQ ID NO: 2 (trastuzumab); b) a heavy chain comprising the amino acid sequence of SEQ ID NO: 3 and a light chain comprising the amino acid sequence of SEQ ID NO: 4 (cetuximab); c) a heavy chain comprising the amino acid sequence of SEQ ID NO: 5 and a light chain comprising the amino acid sequence of SEQ ID NO: 6 (rituximab); d) a heavy chain comprising the amino acid sequence of SEQ ID NO: 7 and a light chain comprising the amino acid sequence of SEQ ID NO: 8 (daratumumab); e) a heavy chain comprising the amino acid sequence of SEQ ID NO: 9 and a light chain comprising the amino acid sequence of SEQ ID NO: 10 (avelumab); f) a heavy chain comprising the amino acid sequence of SEQ ID NO: 59 and a light chain comprising the amino acid sequence of SEQ ID NO: 60 (atezolizumab); g) a heavy chain comprising the amino acid sequence of SEQ ID NO: 61 and a light chain comprising the amino acid sequence of SEQ ID NO: 62 (durvalumab); h) a heavy chain comprising the amino acid sequence of SEQ ID NO: 63 and a light chain comprising the amino acid sequence of SEQ ID NO: 64 (cosibelimab); i) a heavy chain comprising the amino acid sequence of SEQ ID NO: 65 and a light chain comprising the amino acid sequence of SEQ ID NO: 66 (margetuximab); j) a heavy chain comprising the amino acid sequence of SEQ ID NO: 67 and a light chain comprising the amino acid sequence of SEQ ID NO: 68 (pertuzumab); k) a heavy chain comprising the amino acid sequence of SEQ ID NO: 69 and a light chain comprising the amino acid sequence of SEQ ID NO: 70 (enoblituzumab); I) a heavy chain comprising the amino acid sequence of SEQ ID NO: 71 and a light chain comprising the amino acid sequence of SEQ ID NO: 72 (necitumumab); m) a heavy chain comprising the amino acid sequence of SEQ ID NO: 73 and a light chain comprising the amino acid sequence of SEQ ID NO: 74 (panitumumab); n) a heavy chain comprising the amino acid sequence of SEQ ID NO: 75 and a light chain comprising the amino acid sequence of SEQ ID NO: 76 (amivantamab EGFR-binding); o) a heavy chain comprising the amino acid sequence of SEQ ID NO: 77 and a light chain comprising the amino acid sequence of SEQ ID NO: 78 (amivantamab cMet-binding); p) a heavy chain comprising the amino acid sequence of SEQ ID NO: 79 and a light chain comprising the amino acid sequence of SEQ ID NO: 80 (zolbetuximab); q) a heavy chain comprising the amino acid sequence of SEQ ID NO: 81 and a light chain comprising the amino acid sequence of SEQ ID NO: 82 (dinutuximab); r) a heavy chain comprising the amino acid sequence of SEQ ID NO: 83 and a light chain comprising the amino acid sequence of SEQ ID NO: 84 (naxitamab); s) a heavy chain comprising the amino acid sequence of SEQ ID NO: 85 and a light chain comprising the amino acid sequence of SEQ ID NO: 86 (enfortumab); t) a heavy chain comprising the amino acid sequence of SEQ ID NO: 87 and a light chain comprising the amino acid sequence of SEQ ID NO: 88 (farletuzumab); u) a heavy chain comprising the amino acid sequence of SEQ ID NO: 89 and a light chain comprising the amino acid sequence of SEQ ID NO: 90 (tisotumab); v) a heavy chain comprising the amino acid sequence of SEQ ID NO: 91 and a light chain comprising the amino acid sequence of SEQ ID NO: 92 (mirvetuximab); w) a heavy chain comprising the amino acid sequence of SEQ ID NO: 93 and a light chain comprising the amino acid sequence of SEQ ID NO: 94 (treptavidi); x) a heavy chain comprising the amino acid sequence of SEQ ID NO: 95 and a light chain comprising the amino acid sequence of SEQ ID NO: 96 (vobramitamab); y) a heavy chain comprising the amino acid sequence of SEQ ID NO: 97 and a light chain comprising the amino acid sequence of SEQ ID NO: 98, and optionally an Fc chain comprising the amino acid sequence of SEQ ID NO: 99 (onartuzumab); z) a heavy chain comprising the amino acid sequence of SEQ ID NO: 100 and a light chain comprising the amino acid sequence of SEQ ID NO: 101 (olaratumab); and aa) a heavy chain comprising the amino acid sequence of SEQ ID NO: 102 and a light chain comprising the amino acid sequence of SEQ ID NO: 103 (rovalpituzumab).

An Antigen-Binding Region with Affinity for a Surface Antigen Expressed on NK Cells

In one embodiment, a multispecific antigen binding protein as described herein, can further comprise a second antigen-binding region, which is an antigen-binding region that has affinity for a surface antigen expressed on NK cells. Thus, the presence of the second antigen-binding region with affinity for a surface antigen expressed on NK cells in the multispecific antigen binding protein is optional.

In one embodiment of the multispecific antigen binding protein, the second antigen-binding region that has affinity for a surface antigen expressed on NK cells comprises or consists of an immunoglobulin Fc region, or at least a portion thereof that binds the type III Fcγ receptor (FcγRIIIa) as expressed on (human) NK cells, also referred to herein as CD16A. In one embodiment, the immunoglobulin Fc region at least comprises at least one of a CH2 and CH3 domain. In one embodiment, the immunoglobulin Fc region at least comprises at least one of a CH2 and CH3 domain and a hinge region. In one embodiment, the immunoglobulin Fc region comprises or consists of a hinge region and a CH2 and CH3 domain. In one embodiment, the immunoglobulin Fc region is a dimeric Fc region or at least a portion thereof that binds CD16A.

In one embodiment, an Fc region or portion thereof that binds CD16A be a wild-type region or portion thereof.

In one embodiment, an Fc region or portion thereof that binds CD16A can be modified to enhance or reduce its binding affinity to CD16A. Within the Fc region, CD16A binding is mediated by the hinge region and the CH2 domain. For example, within human IgG1, the interaction with CD16 is primarily focused on amino acid residues D265-E269, N297-T299, A327-1332, L 234-S239, and carbohydrate residue N-acetyl-D-glucosamine in the CH2 domain (see, Sondermann et al., 2000 Nature, 406(6793):267-273). Based on the known domains, mutations can be selected to enhance or reduce the binding affinity to CD16A, such as by using phage-displayed libraries or yeast surface-displayed cDNA libraries or can be designed based on the known three-dimensional structure of the interaction. In one embodiment, the Fc region or portion is IgG2.

Thus, in one embodiment, where a multispecific antigen binding protein is intended to have increased affinity for CD16A, an Fc region or portion thereof that binds CD16A, can comprise a modification to increase affinity for CD16A. Thus, an Fc region or portion thereof that binds CD16A, can comprise one or more amino acid modifications (e.g. amino acid substitutions, deletions, insertions) which increase binding to (human) CD16A and optionally another receptor such as FcRn. Typical modifications include modified human IgG1-derived constant regions comprising at least one amino acid modification (e.g. substitution, deletions, insertions), and/or altered types of glycosylation, e.g., hypofucosylation. A modification can, for example, increase binding of an Fc region to FcγRIIIa (CD16A) on NK cells. Examples of modifications are provided in U.S. Pat. No. 10,577,419, the disclosure of which is incorporated herein by reference. Specific mutations (in IgG1 Fc regions) which enhance FcγRIIIa (CD16A) binding, include E333A, S239D/1332E and S239D/A330L/1332E.

In one embodiment, the multispecific antigen binding protein comprises an Fc region or portion thereof that binds CD16A comprising at least one amino acid modification (for example, 1, 2, 3, 4, 5, 6, 7, 8, 9, or more amino acid modifications) relative to a wild-type Fc region, such that the molecule has enhanced binding affinity for (human) CD16A relative to a molecule comprising a wild-type Fc region, optionally wherein the variant Fc region comprises a substitution at any one or more of positions 239, 298, 330, 332, 333 and/or 334 (e.g. S239D, S298A, A330L, 1332E, E333A and/or K334A substitutions), optionally wherein the variant Fc region comprises a substitution at residues S239 and 1332, e.g. a S239D and 1332E substitution (Kabat EU numbering).

In one embodiment, the multispecific antigen binding protein comprises an Fc region or portion thereof that binds CD16A comprising altered glycosylation patterns that increase binding affinity for (human) CD16A. Such carbohydrate modifications can be accomplished by, for example, by expressing a nucleic acid encoding the multispecific protein in a host cell with altered glycosylation machinery. Cells with altered glycosylation machinery are known in the art and can be used as host cells in which to express recombinant antibodies to thereby produce an antibody with altered glycosylation. See, for example. Shields, R. L. et al. (2002) J. Biol. Chem. 277:26733-26740; Umana et al. (1999) Nat. Biotech. 17:176-1, as well as, European Patent No: EP 1,176,195; WO 06/133148; WO 03/035835; WO 99/54342, each of which is incorporated herein by reference in its entirety. In one embodiment, the multispecific antigen binding protein comprises one or more hypofucosylated constant regions. Such a multispecific antigen binding protein can comprise an amino acid alteration or cannot comprise an amino acid alteration and/or may be expressed or synthesized or treated under conditions that result in hypofucosylation. In one embodiment, in a composition comprising the multispecific antigen binding protein described herein, at least 20, 30, 40, 50, 60, 75, 85, 90, 95% or substantially all of the multispecific antigen binding protein have a constant region comprising a core carbohydrate structure (e.g. complex, hybrid and high mannose structures) which lacks fucose. In one embodiment, provided is a multispecific antigen binding protein which is free of N-linked glycans comprising a core carbohydrate structure having fucose.

The core carbohydrate will preferably be a sugar chain at Asn297.

In one embodiment, the multispecific antigen binding protein comprising an Fc region or portion thereof that binds CD16A that is modified to have increased binding affinity for CD16A, has a binding affinity for human CD16A that is at least 1, 2 or 3 log greater than that of a conventional or wild-type human IgG1 antibody, e.g., as assessed by surface plasmon resonance.

In another embodiment, where a multispecific antigen binding protein is intended to have reduced affinity for CD16A, a CH2 and/or CH3 domain, an Fc region or portion thereof that binds CD16A, can comprise a modification to decrease affinity for CD16A. For example, CH2 mutations in a dimeric Fc region protein at reside N297 (Kabat numbering) can eliminate CD16A binding.

Other modification in the Fc region that reduce or eliminate binding to CD16A include the L234A/L235A, also known as “LALA” modifications. Modification of the Fc region that reduce or eliminate its binding to CD16A can be useful in a multispecific antigen binding protein to reduce or avoid NK cell fratricide. The lack of NK cell fratricide can be an advantageous feature for the multispecific antigen binding protein described herein. NK cell cross-linking with NK cells or other immune cells is expected to reduce therapeutic efficacy of NK cell-engagement. Most importantly, cross-linking of a NK cell with one or more NK cells or other immune cells through bivalent or multivalent interactions with FcRy or in combination with a second immune cell antigen (e.g. NKp46, NKG2D, NKp30, SLAMF7 or CD38) can cause immune cell activation. This might lead to induction of target cell-driven fratricide or immune cell killing (e.g. NK-NK cell lysis), ultimately resulting in efficient NK cell depletion in vivo, as previously described for a CD16-directed murine lgG antibody (3G8), the CD38-directed antibody daratumumab and other approaches (Choi et al 2008 Immunology 124 (2) 215-22; DOI: 10.111 I/j.1365-2567.2007.02757.x; Yoshida 2010 Front. Microbiol 1: 128 DOI: 10.3389/fmicb.2010.00128; Wang et al 2018 Clin Cancer Res, 24(16): 4006-4017; DOI: 10.1158/1078-0432.CCR-17-3117; His et al 2008; Nakamura 2013 PNAS; 110(23) 9421-9426; DOI: 10.1073/pnas.1300140110; Breman et al 2018 Front Immunol, 12(9)2940; DOI: 10.3389/fimmu.2018.02940).

The person of skill in the art will appreciate that other configurations for modification of Fc regions can be implemented. For example, substitutions into human IgG1 or IgG2 residues at positions 233-236 and IgG4 residues at positions 327, 330 and 331 were shown to greatly reduce binding to Fcγ receptors and thus ADCC and CDC. Furthermore, Idusogie et aL. (2000) J. Immunol. 164(8):4178-84 demonstrated that alanine substitution at different positions, including K322, significantly reduced complement activation.

In one embodiment, the multispecific antigen binding protein comprises an Fc region or portion thereof that binds CD16A that is modified to have reduced binding affinity for CD16A, has a binding affinity for human CD16A that is at least 1, 2 or 3 log less than that of a conventional or wild-type human IgG1 antibody, e.g., as assessed by surface plasmon resonance.

In one embodiment, the multispecific antigen binding protein comprises an Fc region that has an amino acid sequence having at least 85, 90, 91, 92, 93, 94, 95, 96, 97, 98, 99 or 100% amino acid identity with an Fc region in at least one of SEQ ID NOs: 1, 3, 5, 7, 9, 11-19 and 23, and preferably having one or more of the above structural and/or functional features.

In one embodiment of the multispecific antigen binding protein, the second antigen-binding region that has affinity for a surface antigen expressed on NK cells comprises or consists of an antigen-binding region that specifically binds a surface antigen expressed on NK cells. The surface antigen expressed on NK cells preferably is an NK cell activating receptor. The (second) antigen-binding region can be an antigen-binding region as described herein above. The (second) antigen-binding region preferably specifically binds NK cell activating receptor selected from the group consisting of: NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, CRACC, 4-1BB, OX40, CRTAM, CD27, PSGL1, CD96, CD100, CEACAM1, and NTB-A, of which NKp46, NKp30, NKG2D, CD16A, CD59, PD-L1, Tim3 and SLAMF7 are preferred.

In one embodiment, the multispecific antigen binding protein is a protein wherein the third antigen-binding region comprises or consists of an antigen-binding region that specifically binds an NK cell activating receptor selected from the group consisting of: NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, CRACC, 4-1BB, OX40, CRTAM, CD27, PSGL1, CD96, CD100, CEACAM1, CD59, PD-L1, Tim3 and NTB-A. In one embodiment, the third antigen-binding region activates the NK cell activating receptor.

“NKp46” refers to a protein or polypeptide encoded by an Ncr1 gene or by a cDNA prepared from such a gene. NKp46 has also been designated as NCR1, CD335 (cluster of differentiation, NKP46, NK-p46, and LY94. Any naturally occurring isoform, allele, ortholog or variant is encompassed by the term NKp46 polypeptide (e.g., an NKp46 polypeptide 90%, 95%, 98% or 99% identical to SEQ ID NO: 50, or a contiguous sequence of at least 20, 30, 50, 100 or 200 amino acid residues thereof). The 304 amino acid residue sequence of human NKp46 (isoform a) is shown in in SEQ ID NO: 50, which corresponds to NCBI accession number NP_004820, the disclosure of which is incorporated herein by reference. The human NKp46 mRNA sequence is described in NCBI accession number NM_004829, the disclosure of which is incorporated herein by reference.

“NKp44” refers to a protein or polypeptide encoded by an Ncr2 gene or by a cDNA prepared from such a gene. NKp44 has also been designated as NCR2, CD336 (cluster of differentiation 336), NKP44, NK-p44, LY95, and dJ149M18.1. Any naturally occurring isoform, allele, ortholog or variant is encompassed by the term NKp44 polypeptide (e.g., an NKp44 polypeptide 90%, 95%, 98% or 99% identical to SEQ ID NO: 51, or a contiguous sequence of at least 20, 30, 50, 100 or 200 amino acid residues thereof). The 276 amino acid residue sequence of human NKp46 is shown in SEQ ID NO: 51, which corresponds to NCBI accession number NP_004819, the disclosure of which is incorporated herein by reference. The human NKp46 mRNA sequence is described in NCBI accession number NM_004828, the disclosure of which is incorporated herein by reference.

“NKp30” refers to a protein or polypeptide encoded by an Ncr3 gene or by a cDNA prepared from such a gene. NKp30 has also been designated as NCR3 and CD337 (cluster of differentiation 337). Any naturally occurring isoform, allele, ortholog or variant is encompassed by the term NKp30 polypeptide (e.g., an NKp30 polypeptide 90%, 95%, 98% or 99% identical to SEQ ID NO:52, or a contiguous sequence of at least 20, 30, 50, 100 or 200 amino acid residues thereof). The 201 amino acid residue sequence of human NKp30 is shown in below in SEQ ID NO: 52, which corresponds to NCBI accession number NP_667341, the disclosure of which is incorporated herein by reference.

The human NKp30 mRNA sequence is described in NCBI accession number NM_147130, the disclosure of which is incorporated herein by reference.

“NKG2D” is an activating receptor (transmembrane protein) belonging to the NKG2 family of C-type lectin-like receptors. NKG2D is encoded by KLRK1 gene in humans. NKG2D recognizes induced-self proteins from MIC and RAET1/ULBP families which appear on the surface of stressed, malignant transformed, and infected cells. “NKG2D” refers to a protein or polypeptide encoded by a KLRK1 gene or by a cDNA prepared from such a gene. NKG2D has also been designated as KLRK1, CD314 (cluster of differentiation 314), D12S2489E, KLR, NKG2-D, natural killer group 2D, killer cell lectin-like receptor K1, killer cell lectin like receptor K1. Any naturally occurring isoform, allele, ortholog or variant is encompassed by the term NKG2D polypeptide (e.g., an NKG2D polypeptide 90%, 95%, 98% or 99% identical to SEQ ID NO: 53, or a contiguous sequence of at least 20, 30, 50, 100 or 200 amino acid residues thereof). The 216 amino acid residue sequence of human NKG2D is shown in SEQ ID NO: 53, which corresponds to NCBI accession number NP_001186734, the disclosure of which is incorporated herein by reference. The human NKG2D mRNA sequence is described in NCBI accession number NM_007360, the disclosure of which is incorporated herein by reference.

“DNAM-1” is a ˜65 kDa glycoprotein expressed on the surface of amongst others NK cells. It is a member of the immunoglobulin superfamily containing 2 Ig-like domains of the V-set. DNAM-1 mediates cellular adhesion to other cells bearing its ligands, CD112 and CD155, and cross-linking DNAM-1 with antibodies causes cellular activation. “DNAM-1” refers to a protein or polypeptide encoded by a CD226 gene or by a cDNA prepared from such a gene. DNAM-1 has also been designated as CD226 (cluster of differentiation 226), DNAM-1, DNAM1, PTA1 and TIiSA1. Any naturally occurring isoform, allele, ortholog or variant is encompassed by the term DNAM-1 polypeptide (e.g., an DNAM-1 polypeptide 90%, 95%, 98% or 99% identical to SEQ ID NO: 54, or a contiguous sequence of at least 20, 30, 50, 100 or 200 amino acid residues thereof). The 336 amino acid residue sequence of human DNAM-1 is shown in SEQ ID NO: 54, which corresponds to NCBI accession number NP_006557, the disclosure of which is incorporated herein by reference. The human DNAM-1 mRNA sequence is described in NCBI accession number NM_006566, the disclosure of which is incorporated herein by reference.

As indicated above, “CD16A” is an immunoglobulin gamma Fc region receptor (FcγRIIIa) that is expressed on NK cells and through which NK cells recognize IgG that is bound to the surface of a pathogen-infected or TAA-expressing target cell. Any naturally occurring isoform, allele, ortholog or variant is encompassed by the term CD16A polypeptide (e.g., an CD16A polypeptide 90%, 95%, 98% or 99% identical to SEQ ID NO: 55, or a contiguous sequence of at least 20, 30, 50, 100 or 200 amino acid residues thereof). The 254 amino acid residue sequence of human CD16A is shown in SEQ ID NO: 55, which corresponds to UniProt accession no. P08637, the disclosure of which is incorporated herein by reference.

“SLAMF7” is a protein that in humans is encoded by the human SLAMF7 gene. Isoform 1 SLAMF7 mediates NK cell activation through a SH2D1A-independent extracellular signal-regulated ERK-mediated pathway. SLAMF7 has also been designated as CD319 (cluster of differentiation 319), 19A, CRACC, and CS1. Any naturally occurring isoform, allele, ortholog or variant is encompassed by the term SLAMF7 polypeptide (e.g., a SLAMF7 polypeptide 90%, 95%, 98% or 99% identical to SEQ ID NO: 56, or a contiguous sequence of at least 20, 30, 50, 100 or 200 amino acid residues thereof). The 335 amino acid residue sequence of human SLAMF7 is shown in SEQ ID NO: 56, which corresponds to UniProt accession no. Q9NQ25-1, the disclosure of which is incorporated herein by reference.

In one embodiment of a multispecific antigen binding protein described herein, the antigen-binding region that specifically binds an NK cell activating receptor is an agonistic antigen-binding region that activates the NK cell receptor. As used herein, an antigen-binding region that has “agonist” activity at an NK cell activating receptor is an agent that can cause or increase “signaling by the NK cell activating receptor”. “Signaling by the NK cell activating receptor” refers to an ability of an NK cell activating receptor to activate or transduce an intracellular signaling pathway.

Changes in NK cell activating receptor-signaling activity can be measured, for example, by assays designed to measure changes in NK cell activating receptor-signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of NK cell activating receptor-sensitive promoters and enhancers, or indirectly by a downstream effect mediated by the NK cell activating receptor polypeptide (e.g. activation of specific cytolytic machinery in NK cells). Reporter genes can be naturally occurring genes (e.g. monitoring cytokine production) or they can be genes artificially introduced into a cell. Other genes can be placed under the control of such regulatory elements and thus serve to report the level of NK cell activating receptor-signaling activity.

Many examples of monoclonal antibodies against NK cell activating receptor have been described in the art. Anti-NKp46 monoclonal antibodies are described WO 2011/086179, WO 2016/209021 and in Gauthier et al. (2019, Cell 177, 1701-1713) or in WO 2016/207278, such as NKp46-1, -2, -3, -4, -6 or -9. Antigen-binding regions that specifically bind NKp46, including their variable domain and CDR sequences, are for example described in WO 2016/207278 and include heavy and light chain sequences of SEQ ID NO.'s: 57 and 58, respectively. Anti-NKG2D monoclonal antibodies described WO 2009/077483, WO 2018/148447, WO 2019/157366, WO 2018/148445, WO 2018/152518 and WO 2019/195409, which include the heavy and light chain sequences of SEQ ID NO.'s: 16 and 20, respectively. Monoclonal antibodies against NKG2A are e.g. described in WO 2008/009545, WO 2009/092805, WO 2016/032334, WO 2020/094071 and WO 2020/102501. Monoclonal antibodies against NKp30 are e.g. described in WO 2020/172605. Monoclonal antibodies against DNAM-1 are e.g. described in WO 2013/140787. Examples of anti-SLAMF7 monoclonal antibodies include Elotuzumab and others described in US2018208653.

Monoclonal antibodies against 4-1BB (CD137) are e.g. described in WO 2005/035584, WO 2006/088464 and US2006188439. Monoclonal antibodies against OX40 are e.g. described in WO 2007/062245, US2010136030, US2019100596, WO 2013/008171 and WO 2013/028231.

Monoclonal antibodies against CD96 are e.g. described in WO 2019/091449. Monoclonal antibodies against CD160 are e.g. described in US2012003224 and US2013122006. Monoclonal antibodies against KIR2DS1-5 are e.g. described in WO 2016/031936.

### NK Cell-Activating Cytokines

A multispecific antigen binding protein as described herein, thus further comprises at least one NK cell-activating cytokine. In one embodiment, NK cell-activating cytokine that is at least one of: i) an interleukin 21 receptor (IL21R) agonist; and, ii) a 4-1BB agonist. In one embodiment, the multispecific antigen binding protein at least comprises an IL21R agonist. In one embodiment, the multispecific antigen binding protein at least comprises a 4-1BB agonist. And, in one embodiment, the multispecific antigen binding protein at least comprises both an IL21R agonist and a 4-1BB agonist. In further embodiments, the multispecific antigen binding protein as described herein can comprises further NK cell-activating cytokines, in addition to at least one of an IL21R agonist and a 4-1 BB agonist. Such further NK cell-activating cytokines can be selected from the group consisting of an IL15 receptor agonist, an IL2 receptor agonist, a type I interferon (IFN-1) agonist, an IL12 receptor agonist and an IL18 receptor agonist, as further detailed below.

Thus, in one embodiment, the multispecific antigen binding protein as described herein at least comprises an interleukin 21 receptor (IL21R) agonist.

Interleukin 21 (IL21) is a protein that in humans is encoded by the/L21 gene (Entrez Gene ID: 59067). IL21 is a cytokine that has potent regulatory effects on cells of the immune system, including natural killer (NK) cells and which induces cell division/proliferation in its target cells. Amino acid sequences for human IL21 precursor (including its signal sequence) are described in NCBI accession numbers NP_001193935 and NP_068575, the disclosures of which are incorporated herein by reference. IL21 (mature/processed) comprises amino acids 30-153 of NP_001193935 or amino acids 30-162 of NP_068575 (i.e. SEQ ID NO: 38). IL21 exerts its effects on target cells through the IL-21 receptor (IL21R) is expressed on the surface of T, B and NK cells. IL21R is similar in structure to the receptors for othertype I cytokines like IL-2R or IL-15 and requires dimerization with the common gamma chain (γc) in order to bind IL-21. IL21R is encoded in humans by the IL21R gene (Entrez Gene ID: 50615). Amino acid sequences for human IL21R are described in NCBI accession numbers NP_068570, NP_851564 and NP_851565, the disclosures of which are incorporated herein by reference.

As used herein, an “IL21R agonist” is an agent that has “agonist” activity at the IL21 receptor, which means that the agent that can cause or increase “IL21R signaling”. “IL21R signaling” refers to an ability of IL21R, e.g. when expressed on the surface of T, B and NK cells and triggered by its natural ligand IL21, to activate or transduce an intracellular signaling pathway. The “natural ligand IL21” is herein understood as a human wild type IL21 comprising or consisting of an amino acid sequence as indicated above. When bound to IL-21, the IL-21 receptor acts through the Jak/STAT pathway, utilizing Jak1 and Jak3 and a STAT3 homodimer to activate its target genes. IL21R agonist activity, i.e. changes in IL21R signaling activity, can be measured, for example, by assays designed to measure changes in the IL21R signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of IL21R-sensitive promoters and enhancers, or indirectly by a downstream effect mediated by IL21R (e.g. activation of specific cytolytic machinery in NK cells). A suitable cell-based assay for biological activity of an IL21R agonist, is e.g. described in Maurer et al. (mAbs. 2012; 4(1): 69-83.), wherein a murine pre-B-cell line is transfected with both the human IL21R and a STAT-responsive luciferase reporter gene. IL21R agonist activity can be determined using this cell line by measuring the level of STAT3 phosphorylation using anti-pSTAT3 antibody-conjugated beads and/or by detecting luciferase luminescence, upon contacting the cell line with an IL21R agonist. The natural ligand IL21 can serve as a positive control in an assay for IL21R agonist activity and can also be used as a reference for the amount of IL21R agonist activity of a given non-natural IL21R agonist, such as a multispecific antigen binding protein as described herein comprising an IL21R agonist.

In one embodiment, a multispecific antigen binding protein as described herein comprises an IL21R agonist that has reduced IL21R agonist activity as compared to human wild type IL21. In one embodiment, the IL21R agonist has an IL21R agonist activity that is a factor 2, 5, 10, 20, 50, 100, 200, 500, 1000, 10000, or 100000 less than that of human wild type IL21.

In one embodiment, a multispecific antigen binding protein as described herein comprises an IL21R agonist that has enhanced IL21R agonist activity as compared to human wild type 1L21. In one embodiment, the IL21R agonist has an IL21R agonist activity that is a factor 2, 5, 10, 20, 50, 100, 200, 500, 1000, 10000, or 100000 higher than that of human wild type IL21.

In one embodiment, a multispecific antigen binding protein as described herein comprises an IL21R agonist that is an IL21 polypeptide comprising an amino acid sequence with at least 50, 55, 60, 65, 70, 75, 80, 85, 90, 95, 96, 97, 98, 99, or 100% sequence identity to SEQ ID NO: 38, and preferably having an IL21R agonist activity as defined above, and/or preferably having an affinity for the IL21R as defined below.

In one embodiment, a multispecific antigen binding protein as described herein comprises an IL21R agonist of which the affinity for the IL21R is reduced or enhanced as compared to human wild type IL21. The affinity of an IL21R agonist of the affinity for the IL21R can be assayed using methods generally known in the art, such as surface plasmon resonance.

In one embodiment, a multispecific antigen binding protein as described herein comprises an IL21R agonist that has reduced affinity for IL21R as compared to human wild type IL21. In one embodiment, the affinity of the IL21R agonist for IL21R is a factor 2, 5, 10, 20, 50, 100, 200, 500 or 1000 less than that of human wild type IL21.

In one embodiment, a multispecific antigen binding protein as described herein comprises an IL21R agonist that has enhanced affinity for IL21R as compared to human wild type IL21. In one embodiment, the affinity of the IL21R agonist for IL21R is a factor 2, 5, 10, 20, 50, 100, 200, 500 or 1000 higher than that of human wild type IL21.

In one embodiment, a multispecific antigen binding protein as described herein comprises an IL21R agonist that is IL21 or a fragment thereof that has IL21R agonist activity. Preferably, the IL21R agonist is human IL21 or a fragment thereof that has IL21R agonist activity. In one embodiment, the IL21R agonist is an IL21 mutein with reduced affinity for IL21R as compared to human wild type IL21. IL21 muteins with reduced affinity for IL21R as compared to human wild type IL21 are described in Shen et al. (Front Immunol. 2020; 11: 832). Thus, in one embodiment, the IL21R agonist is an IL21 mutein with a mutation (i.e. amino acid substitution, deletion or insertion) of one or more amino acids selected from the group consisting of 116, 166, 18, K72, K73, K75, K77, L13, P78, Q12, Q19, R5, R65, R76, R9, S70, S80, V69 and Y23 (amino acid positions referring to position in SEQ ID NO: 38 or a corresponding position in an IL-21 allelic variant). Preferably, the IL21R agonist is an IL21 mutein comprising one or more amino acid substitutions selected from the group consisting of 18A, K72D, K73A, K75D, K77D, L13D, P78D, Q12A, Q19D, R5A, R65D, R76A, R9A, S70E, S80G, V69D, Y23D, 116E, 166G, 18D, K72G, K73D, K75G, K77G, P79D, Q12D, R5D, R65G, R76D, R9D, S70G, S80P, V69G, 166P, 18E, K72P, K73E, K75P, K77P, Q12E, R5E, R65P, R76E, R9E, S70P, V69P, 18G, K73G, Q12N, R5G, R76G, R9G, S70Y, 18N, K73H, Q12S, R5H, R76H, R9H, 18S, K731, Q12T, R51, R761, R91, K73N, Q12V, R5K, R76K, R9K, K73P, R5L, R76L, R9L, K73Q, R5M, R76M, R9M, K73S, R5N, R76N, R9N, K73V, R5Q, R76P, R9Q, R5S, R76Q, R9S, R5T, R76S, R9T, R5V, R76T, R9V, R5Y, R76V, R9Y and R76Y.

In one embodiment, the IL21R agonist is an IL21 mutein that has an affinity for human IL21R-Fc in Table 2 of Shen et al. (2020; supra) between 0.028 and 0.099 nM. In one embodiment, the IL21R agonist is an IL21 mutein that has an affinity for human IL21R-Fc in Table 2 of Shen et al. (2020; supra) between 0.10 and 0.29 nM. In one embodiment, the IL21R agonist is an IL21 mutein that has an affinity for human IL21R-Fc in Table 2 of Shen et al. (2020; supra) between 0.30 and 0.99 nM. In one embodiment, the IL21R agonist is an IL21 mutein that has an affinity for human IL21R-Fc in Table 2 of Shen et al. (2020; supra) between 1.0 and 2.9 nM. In one embodiment, the IL21R agonist is an IL21 mutein that has an affinity for human IL21R-Fc in Table 2 of Shen et al. (2020; supra) >2.9 nM.

In one embodiment, a multispecific antigen binding protein as described herein comprises an IL21R agonist that is an antigen-binding region that specifically binds IL21R and that has IL21R agonist activity. The antigen-binding region can be an antigen-binding region as described herein above.

In one embodiment, a multispecific antigen binding protein as described herein comprises more than one IL21R agonist as described above. Thus, in one embodiment, the multispecific antigen binding protein has an IL21R agonist-valency that is higher than one. The IL21R agonist-valency of a multispecific antigen binding protein can for example be 2, 3, 4, 5, 6, 7, 8, 9, 10, 11, 12, 13, 14, 15 or more.

In one embodiment, the multispecific antigen binding protein as described herein at least comprises a 4-1 BB agonist.

4-1BB is a member of the tumor necrosis factor receptor family. Its alternative names are tumor necrosis factor receptor superfamily member 9 (TNFRSF9), CD137 and induced by lymphocyte activation (ILA). 4-1 BB is encoded by the TNFRSF9 gene (Entrez Gene ID: 3604). An amino acid sequence for human 4-1BB is described in NCBI accession numbers NP_001552, the disclosure of which is incorporated herein by reference. 4-1 BB is known as a co-stimulatory immune checkpoint molecule. 4-1 BB is expressed by activated T cells of both the CD4+ and CD8+ lineages, as well as on activated NK cells. NK cells with increased 4-1 BB expression are known to be highly active against target cells (e.g. tumor cells) expressing 4-1 BB ligand. 4-1 BB ligand (4-1 BBL), also known as TNFSF9 or CD137L, is a protein that in humans is encoded by the TNFSF9 gene (Entrez Gene ID: 8744). An amino acid sequence for human 4-1BBL is described in NCBI accession numbers NP_003802, the disclosure of which is incorporated herein by reference. The 4-11BB/4-1BBL complex consists of three monomeric 4-1 BBs bound to a trimeric 4-1BBL. Each 4-1BB monomer binds to two 4-1 BBLs via cysteine-rich domains (CRDs). The interaction between 4-1 BB and the second 4-1 BBL is required to stabilize their interactions.

As used herein, an “4-1 BB agonist” is an agent that has “agonist” activity at the 4-1 BB, which means that the agent that can cause or increase “4-1 BB signaling”. “4-1 BB signaling” refers to an ability of 4-1 BB, e.g. when expressed on the surface of T, B and NK cells and triggered by its natural ligand 4-1BBL, to activate or transduce an intracellular signaling pathway. The “natural 4-1BB ligand” is herein understood as the extracellular domain (ECD) of a human wild type 4-1BBL comprising or consisting of an amino acid sequence from position 71 to 254 of the amino acid sequence of human 4-1 BBL (i.e. SEQ ID NO: 37). A 4-1 BBL extracellular domain (ECD) is herein thus understood as a polypeptide comprising or consisting of an amino acid sequence from positions 71 to 254 of human 4-1 BBL, or a fragment thereof having 4-1 BB agonist activity.

4-1BB agonist activity, i.e. changes in 4-1BB signaling activity, can be measured, for example, by assays designed to measure changes in the 4-1BB signaling pathways, e.g. by monitoring phosphorylation of signal transduction components, assays to measure the association of certain signal transduction components with other proteins or intracellular structures, or in the biochemical activity of components such as kinases, or indirectly by a downstream effect mediated by 4-1 BB (e.g. production of specific cytokines). A suitable cell-based assay for in vitro biological activity of a 4-1BB agonist, is e.g. described in Zhang et al. (Clin Cancer Res,2007; 13(9): 2758-2767), using measurement of IL-2 production from splenocytes aseptically removed from BALB/c mice in microtiter plates precoated with an anti-CD3 monoclonal antibody (145-11C clone). Other suitable cell-based assays for in vitro biological activity of a 4-1BB agonist, are described in WO2016/075278, Example 6 (see e.g. Example 6.1). The natural 4-1BB ligand, a 4-1BBL ECD trimer as described by Fellermeier et al. (Oncoimmunol. 2016, 5(11): e1238540), e.g. a 4-1BBL ECD trimer comprising the amino acid sequence of SEQ ID NO: 36, or an anti-CD137 agonist antibody (such the antibody 2A, Epstein et al., Tumor necrosis imaging and treatment of solid tumors. In: V. P. Torchilin, editor. Handbook of targeted delivery of imaging agents, Vol. 16. Boca Raton: CRCPress; 1995. p. 259.) can serve as a positive control in an assay for 4-1BB agonist activity and can also be used as a reference for the amount of 4-1BB agonist activity of a given non-natural 4-1BB agonist, such as a multispecific antigen binding protein as described herein comprising a 4-1 BB agonist.

In one embodiment, a multispecific antigen binding protein as described herein comprises a 4-1 BB agonist that has reduced 4-1 BB agonist activity as compared to human wild type 4-1 BBL or the anti-4-1BB agonist antibody 2A. In one embodiment, the 4-1BB agonist has a 4-1BB agonist activity that is a factor 2, 5, 10, 20, 50, 100, 200, 500 or 1000 less than that of the ECD of human wild type 4-1BBL or the anti-4-1BB agonist antibody 2A.

In one embodiment, a multispecific antigen binding protein as described herein comprises a 4-1 BB agonist that has enhanced 4-1 BB agonist activity as compared to human wild type 4-1 BBL.

In one embodiment, the 4-1 BB agonist has a 4-1 BB agonist activity that is a factor 2, 5, 10, 20, 50, 100, 200, 500 or 1000 higher than that of the ECD of human wild type 4-1 BBL or the anti-4-1 BB agonist antibody 2A.

In one embodiment, a multispecific antigen binding protein as described herein comprises a 4-1BB agonist comprising at least one 4-1BBL ECD comprising an amino acid sequence with at least 50, 55, 60, 65, 70, 75, 80, 85, 90, 95, 96, 97, 98, 99, or 100% sequence identity to SEQ ID NO: 37, and preferably having an 4-1 BB agonist activity as defined above, and/or preferably having an affinity for 4-1 BB as defined below.

In one embodiment, a multispecific antigen binding protein as described herein comprises a 4-1 BB agonist of which the affinity for the 4-1 BB is reduced or enhanced as compared to the ECD of human wild type 4-1 BBL. The affinity of a 4-1 BB agonist of the affinity for the 4-1 BB can be assayed using methods generally known in the art, such as surface plasmon resonance.

In one embodiment, a multispecific antigen binding protein as described herein comprises a 4-1 BB agonist that has reduced affinity for 4-1 BB as compared to human wild type 4-1 BBL. In one embodiment, the affinity of the 4-1 BB agonist for 4-1 BB is a factor 2, 5, 10, 20, 50, 100, 200, 500 or 1000 less than that of the ECD of human wild type 4-1 BBL or the anti-4-1 BB agonist antibody 2A.

In one embodiment, a multispecific antigen binding protein as described herein comprises a 4-1BB agonist that has enhanced affinity for 4-1BB as compared to human wild type 4-1BBL. In one embodiment, the affinity of the 4-1 BB agonist for 4-1 BB is a factor 2, 5, 10, 20, 50, 100, 200, 500 or 1000 higher than that of the ECD of human wild type 4-1 BBL or the anti-4-1 BB agonist antibody 2A.

In one embodiment, a multispecific antigen binding protein as described herein comprises a 4-1 BB agonist that comprises or consists of the ECD of 4-1 BBL or a fragment thereof that has 4-1 BB agonist activity. Preferably, the 4-1 BB agonist is human 4-1 BBL or a fragment thereof that has 4-1BB agonist activity. In one embodiment, the 4-1BB agonist is a mutein of the ECD of 4-1BBL with reduced affinity for 4-1 BB as compared to the ECD of human wild type 4-1 BBL or the anti-4-1 BB agonist antibody 2A.

In one embodiment, a multispecific antigen binding protein as described herein comprises a 4-1BB agonist that comprises or consists of a fusion protein comprising three 4-1BBL ECD monomers fused together in a single polypeptide chain, as e.g. described in Fellermeier et al. (2016, supra). In one embodiment, the three 4-1 BBL ECD monomers are connected by polypeptide linkers.

In one embodiment, the three 4-1BBL ECD monomers are connected by polypeptide linkers selected from the group consisting of (GGGGS)₄, GGGSGGG, GGSGGGGSGG and G, of which (GGGGS)₄ preferred. Other suitable flexible polypeptide linker(s) are described herein below. In one embodiment, a multispecific antigen binding protein as described herein comprises a 4-1BB agonist that comprises or consists of a fusion protein comprising three 4-1BBL ECD monomers fused together in a single polypeptide chain, e.g. comprising the amino acid sequence of SEQ ID NO: 36.

In one embodiment, a multispecific antigen binding protein as described herein comprises a 4-1BB agonist that comprises three 4-1BBL ECD monomers that are present in more than one polypeptide chain of the multispecific antigen binding protein. For example, two 4-1BBL ECD monomers can be fused together in a single polypeptide chain, optionally connected together through polypeptide linker as described above, that is part of a first polypeptide chain of the multispecific antigen binding protein and the third 4-1BBL ECD monomer is part of a second polypeptide chain of the multispecific antigen binding protein as e.g. described in WO2016/075278.

The first and second polypeptide chains of the multispecific antigen binding protein can be the chains comprising the heavy and light chains, respectively, or vice versa, whereby preferably the 4-1 BBL ECDs are fused to the N-termini of the variable domains. Alternatively, the first and second polypeptide chains of the multispecific antigen binding protein can be the two chains comprising the two heavy chains, whereby preferably the 4-1 BBL ECDs are fused to the C-termini of the constant domains.

In one embodiment, a multispecific antigen binding protein as described herein comprises a 4-1 BB agonist that comprises a antigen-binding region that specifically binds 4-1 BB and that has 4-1BB agonistic activity. Antibodies against 4-1BB are e.g. described in WO2005035584, WO2006088464, US2006188439.

In one embodiment, a multispecific antigen binding protein as described herein comprises more than one 4-1BB agonist as described above. Thus, in one embodiment, the multispecific antigen binding protein has a 4-1 BB agonist-valency that is higher than one. The 4-1 BB agonist-valency of a multispecific antigen binding protein can for example be 2, 3, 4, 5, 6, 7, 8, 9, 10, 11, 12, 13, 14, 15 or more.

In a preferred embodiment, a multispecific antigen binding protein as described herein comprises both at least one IL21R agonist as herein described above and at least one 4-1BB agonist as herein described above.

In further embodiments, the multispecific antigen binding protein as described herein can comprise, in addition to at least one of an IL21R agonist and a 4-1 BB agonist, a further NK cell-activating cytokines selected from the group consisting of an IL15 receptor agonist, an IL2 receptor agonist, an IL12 receptor agonist and an IL18 receptor agonist. In one embodiment, the IL15 receptor agonist is an IL15 polypeptide or an agonistic antigen-binding region that specifically binds the IL15 receptor. In one embodiment, the IL2 receptor agonist is an IL2 polypeptide or an agonistic antigen-binding region that specifically binds the IL2 receptor. In one embodiment, the IL12 receptor agonist is an IL12 polypeptide or an agonistic antigen-binding region that specifically binds the IL12 receptor. In one embodiment, the IL18 receptor agonist is an IL18 polypeptide or an agonistic antigen-binding region that specifically binds the IL18 receptor.

### Structure of the Multispecific Antigen Binding Protein

In one embodiment, in a multispecific antigen binding protein as described herein, the at least one of the first and third antigen-binding regions that specifically binds a TAA is conjugated to the second antigen-binding region that has affinity for a surface antigen expressed on NK cells.

Preferably, the at least one of the first and third antigen-binding regions that specifically binds a TAA is conjugated to at least one polypeptide chain of the second antigen-binding region. The conjugation of the two domains/regions is understood to mean that they are covalently linked to each other. The two domains/regions can be chemically cross-linked to each other, using a cross-linker for linking two proteinaceous molecules, as are well-known in the art. There are a number of commercially available crosslinking reagents for preparing protein or peptide bioconjugates. Many of these crosslinkers allow dimeric homo- or heteroconjugation of biological molecules through free amine or sulfhydryl groups in protein side chains. Other crosslinking methods involve coupling through carbohydrate groups with hydrazide moieties. For cross-linking a TAA binding regions to a NK cell-binding region it is preferred that cross-linkers with heterofunctional specificity are used. In one embodiment, the cross-linker comprises a flexible spacer, to provide flexibility or freedom of motion of the two regions with respect to each other.

In one embodiment, however, the at least one of the first and third antigen-binding regions that specifically binds a TAA is conjugated to the second antigen-binding region that has affinity for a surface antigen expressed on NK cells by being comprised in a single polypeptide chain. As an antigen-binding region can comprise two polypeptide chains, such as a V_{H} and a V_{L} chain, in one embodiment, at least one polypeptide chain in an antigen-binding region that specifically binds a TAA forms a single polypeptide chain with at least one polypeptide chain of the second antigen-binding region. Likewise, as also the second antigen-binding region that has affinity for a surface antigen expressed on NK cells can comprise two polypeptide chains, such as a dimeric Fc region of an antibody, in one embodiment, at least one polypeptide chain of the second antigen-binding region forms a single polypeptide chain with the at least one polypeptide chain in an antigen-binding region that specifically binds a TAA.

Thus, in one embodiment, a multispecific antigen binding protein as described herein comprises a single polypeptide chain that comprises in an N- to C-terminal order: i) at least one polypeptide chain in the at least one of the first and third antigen-binding region that specifically binds a TAA; ii) optionally a flexible linker; and iii) (at least one polypeptide chain of) the second antigen-binding region that has affinity for a surface antigen expressed on NK cells. The flexible linker can be an immunoglobulin hinge region or can be linker as described herein below.

In one embodiment, the domain that has affinity for a surface antigen expressed on NK cells is a dimeric immunoglobulin Fc region, wherein each of the two polypeptide chains of the Fc region is linked to a CH1 domain, each of which CH1 domains is linked to an immunoglobulin variable region that specifically binds a TAA. The dimeric immunoglobulin Fc region preferably is a dimer of an Fc region as herein described above. The immunoglobulin variable region can be an scFv, a V_{H} domain, a V_{L} domain, or an immunoglobulin single variable domain (ISVD) such as a dAb, a V-NAR domain or a V_{H}H domain. In one embodiment, the immunoglobulin variable region that is linked to the CH1 domain is a V_{H} domain that is paired with a V_{L} domain linked to a Cκ or Cλ domain. In this embodiment, preferably, the V_{H} and V_{L} domains together specifically bind the TAA.

In one embodiment, the two immunoglobulin variable regions bind the same TAA, or wherein the two immunoglobulin variable regions each bind a different TAA. With respect to the specificity for a TAA, a multispecific antigen binding protein as described herein can thus be homodimeric, with two identical immunoglobulin variable regions that both bind the same TAA. Alternatively, a multispecific antigen binding protein as described herein can thus be heterodimeric with respect to the specificity for TAAs, wherein each of the two immunoglobulin variable regions each bind a different TAA. In embodiments, wherein multispecific antigen binding protein is bispecific with respect to the TAAs, it is preferred that one of the two immunoglobulin variable regions is an immunoglobulin single variable domain, while the other immunoglobulin variable region is not. Next, the assembly of heterodimeric antibody heavy chains can be accomplished by expressing two different antibody heavy chain sequences in the same cell, which may lead the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, U.S. Ser. No. 14/830,336 and WO2019/195409. For example, mutations can be made in the CH3 domain based on human IgG1 and incorporating distinct pairs of amino acid substitutions within a first polypeptide and a second polypeptide that allow these two chains to selectively heterodimerize with each other. For example, CH3 domains which comprise amino acid substitutions, wherein the CH3 domain interface of the antibody Fc region is mutated to create altered charge polarity across the Fc dimer interface such that co-expression of electrostatically matched Fc chains supports favorable attractive interactions thereby promoting desired Fc heterodimer formation, whereas unfavorable repulsive charge interactions suppress unwanted Fc homodimer formation.

In one embodiment, a “knob-into-holes” approach is used in which the CH3 domain interface of the antibody Fc region is mutated so that the antibodies preferentially form heterodimers (further including the attached light chains). These mutations create altered charge polarity across the Fc dimer interface such that coexpression of electrostatically matched Fc chains support favorable attractive interactions thereby promoting desired Fc heterodimer formation, whereas unfavorable repulsive charge interactions suppress unwanted Fc homodimer formation. For example, one heavy chain comprises a T366W substitution and the second heavy chain comprises a T366S, L368A and Y407V substitution, see, e.g. Ridgway et al (1996) Protein Eng., 9, pp. 617-621; Atwell (1997) J. MoL. Biol., 270, pp. 26-35; and WO2009/089004, the disclosures of which are incorporated herein by reference. In another approach, one heavy chain comprises a F405L substitution, and the second heavy chain comprises a K409R substitution, see, e.g., Labrijn et al. (2013) Proc. Nat/. Acad. Sci. U.S.A., 110, pp. 5145-5150. In another approach, one heavy chain comprises T350V, L351Y, F405A, and Y407V substitutions and the second heavy chain comprises T350V, T366S, K392L, and T394W substitutions, see, e.g. Von Kreudenstein et al., (2013) mAbs 5:646-654. In another approach, one heavy chain comprises both K409D and K392D substitutions and the second heavy chain comprises both D399K and E356K substitutions, see, e.g. Gunasekaran et al., (2010) J. Biol. Chem. 285:19637-19646. In another approach, one heavy chain comprises D221E, P228E and L368E substitutions and the second heavy chain comprises D221R, P228R, and K409R substitutions, see, e.g. Strop et al., (2012) J. MoL. Biol. 420: 204-219. In another approach, one heavy chain comprises S364H and F405A substitutions and the second heavy chain comprises Y349Tand, T394F substitutions, see, e.g. Moore et al., (2011) mAbs 3: 546-557. In another approach, one heavy chain comprises a H435R substitution, and the second heavy chain optionally may or may not comprise a substitution, see, e.g. U.S. Pat. No. 8,586,713. When such hetero-multimeric antibodies have Fc regions derived from a human IgG2 or IgG4, the Fc regions of these antibodies can be engineered to contain amino acid modifications that permit CD16 binding. In some embodiments, the antibody may comprise mammalian antibody-type N-linked glycosylation at residue N297 (Kabat EU numbering).

In one preferred embodiment, a multispecific antigen binding protein as described herein comprises a dimeric immunoglobulin Fc region that is a dimer of an Fc region as herein described above, wherein each of the two Fc polypeptide chains is operably linked to a Fab that specifically binds a TAA. Apart from the presence of the NK cell-activating cytokine(s), a multispecific antigen binding protein comprising such a dimeric Fc linked to two Fabs thus forms an immunoglobulin structure, such as a conventional IgG immunoglobulin.

A multispecific antigen binding protein as described herein further comprises at least one NK cell-activating cytokine. In one embodiment, the at least one of the NK cell-activating cytokine is conjugated to the at least one of the first and a third antigen-binding regions that specifically binds a TAA, or to the second antigen-binding region that has affinity for a surface antigen expressed on NK cells. As indicated above conjugation of two proteinaceous entities is understood to mean that they are covalently linked to each other, which can be done by chemical cross-linking, using cross-linkers for linking two proteinaceous molecules, as are well-known in the art, which cross-linker can comprise a flexible spacer.

In one embodiment, however, the at least one NK cell-activating cytokine forms a single polypeptide chain with at least one of: i) at least one polypeptide chain in the at least one of the first and a third antigen-binding regions that specifically binds a TAA; and, ii) (at least one polypeptide chain in) the second antigen-binding region that has affinity for a surface antigen expressed on NK cells. In one embodiment, a flexible linker (as described below) is present between the agonist and the region defined in i) or ii).

In one embodiment, the at least one NK cell-activating cytokine forms a single polypeptide chain with at least one of: i) a light chain in at least one of the two Fabs that specifically bind a TAA; and, ii) at least one of the two Fc chains in the dimeric immunoglobulin Fc region. In one embodiment, a flexible linker (as described below) is present between the agonist and the light chain defined in i) or the Fc chain defined in ii).

In one embodiment, the at least one NK cell-activating cytokine is fused to at least one of: i) the N-terminus of the light chain in at least one of the two Fabs that specifically bind a TAA, optionally through a flexible linker; ii) the C-terminus of the light chain in at least one of the two Fabs that specifically bind a TAA, optionally through a flexible linker; iii) the N-terminus of the heavy chain in at least one of the two Fabs that specifically bind a TAA; and, iv) the C-terminus of the heavy chain in at least one of the two Fc chains in the dimeric immunoglobulin Fc region, optionally through a flexible linker, whereby the flexible linker can be as described below.

In one embodiment, wherein the multispecific antigen binding protein comprises a (homo- or hetero) dimeric antigen binding protein as described above, the dimer can comprise at least one NK cell-activating cytokine on only one of the two monomers in the dimer, or the dimer can comprise at least one NK cell-activating cytokine on each (i.e. both) of the two monomers in the dimer. Thus, in one embodiment, wherein the multispecific antigen binding protein comprises an immunoglobulin structure, at least one of the NK cell-activating cytokine can be present on at least one or on both sides of the immunoglobulin structure. In embodiments wherein at least one NK cell-activating cytokine is present on each of the two monomers in the dimer or immunoglobulin structure, the multispecific antigen binding protein can for example comprises an IL21R agonist on both monomers, a 4-1 BB agonist on both monomers or, an IL21R agonist on a first monomer and a 4-1BB agonist on a second monomer. It is understood that when the multispecific antigen binding protein comprises heterodimeric heavy chains, the “knob-into-hole” technology as described above can be applied, wherein the CH3 domain of the first chain is modified to have a “protuberance” (“knob”) and the second chain is modified to have a corresponding “cavity” (“hole”).

Thus, in one embodiment, a multispecific antigen binding protein as described herein is heterodimeric with respect to at least one of i) the first and third antigen-binding regions; and ii) at least one fused NK cell-activating cytokine, the dimeric Fc region comprises different first and a second polypeptide chains comprising knob-into-hole modifications promoting association of the first and the second polypeptide chains of the Fc region.

Suitable linker-amino acid sequences for linking the various functional domains and regions in a multispecific antigen binding protein as described herein, are known in the art (e.g. from Chen et al., 2013, Adv Drug Deliv Rev. 65(10): 1357-1369). Linker amino acid sequence can be rigid but are usually flexible. Flexible linkers are usually applied when the joined domains require a certain degree of movement or interaction. They are generally composed of small, non-polar (e.g. Gly) or polar (e.g. Ser or Thr) amino acids. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. Preferred flexible linkers have sequences consisting primarily of stretches of Gly and Ser residues (“GS” linker). An example of preferred (and widely used) flexible linker has the sequence of (GGGGS)ₙ (SEQ ID NO: 30). By adjusting the copy number “n”, the length of this GS linker can be optimized to achieve appropriate separation of the functional domains, or to maintain necessary inter-domain interactions. Specific examples of GS linkers include (GGGGS)₄ (SEQ ID NO: 31), GGGSGGG (SEQ ID NO: 32), GGSGGGGSGG (SEQ ID NO: 33) and G. Besides the GS linkers, many other flexible linkers have been designed for recombinant fusion proteins. These flexible linkers are also rich in small or polar amino acids such as Gly and Ser, but can contain additional amino acids such as Thr and Ala to maintain flexibility, as well as polar amino acids such as Lys and Glu to improve solubility, such as e.g. the flexible linkers KESGSVSSEQLAQFRSLD (SEQ ID NO: 34) and EGKSSGSGSESKST (SEQ ID NO: 35), that have been applied for the construction of a bioactive scFvs.

In one embodiment, a multispecific antigen binding protein as described herein is a multispecific antigen binding protein as exemplified herein, such as e.g. AVC-001, AVC-002, AVC-003, AVC-004, AVC-007 or AVC-008 (see Tables 1.1.1 and 1.1.2) or a derivative thereof wherein the trastuzumab variable heavy (V_{H}) and variable light (V_{L}) domains are replaced by variable heavy (V_{H}) and variable light (V_{L}) domains from another monoclonal antibody against a TAA, for example, from a monoclonal antibody against a TAA known in the art as herein described above.

In one embodiment, a multispecific antigen binding protein as described herein is a multispecific antigen binding protein comprising: a) a first heavy chain of a cytotoxic monoclonal antibody against a TAA as described above, wherein preferably a first NK cell activating cytokine is fused to the C-terminus of the first heavy chain, optionally through a flexible linker; b) a second heavy chain of the cytotoxic monoclonal antibody against the TAA, wherein preferably a second NK cell activating cytokine is fused to the C-terminus of the second heavy chain, optionally through a flexible linker; c) a first light chain and a second light chain of the cytotoxic monoclonal antibody against the TAA as described above, wherein optionally the first or the second NK cell activating cytokine is fused to the C-terminus of the light chain, optionally through a flexible linker, wherein preferably the amino acid sequences of the Fc regions of the first and second heavy chains comprising knob-into-hole modifications promoting association of the first and the second heavy chains. In one embodiment, the first NK cell activating cytokine is a 4-1BB agonist as described above, preferably a fusion protein comprising three 4-1BBL ECD monomers fused together in a single polypeptide chain as described above, or a single 4-1BBL ECD monomer as described above. In one embodiment, the second NK cell activating cytokine is an IL21R agonist as described above, preferably an IL21R polypeptide as described above. In one embodiment, cytotoxic monoclonal antibody against a TAA is trastuzumab.

In one embodiment, the multispecific antigen binding protein (AVC-001) comprises: a) a first heavy chain comprising an amino acid sequence with at least 95, 96, 97, 98, 99 or 100% sequence identity to SEQ ID NO: 11; b) a second heavy chain comprising an amino acid sequence with at least 95, 96, 97, 98, 99 or 100% sequence identity to SEQ ID NO: 12; and c) a first light chain and a second light chain comprising an amino acid sequence with at least 95, 96, 97, 98, 99 or 100% sequence identity to SEQ ID NO: 2.

In one embodiment, the multispecific antigen binding protein (AVC-002) comprises: a) a first heavy chain comprising an amino acid sequence with at least 95, 96, 97, 98, 99 or 100% sequence identity to SEQ ID NO: 13; b) a second heavy chain comprising an amino acid sequence with at least 95, 96, 97, 98, 99 or 100% sequence identity to SEQ ID NO: 12; and c) a first light chain and a second light chain comprising an amino acid sequence with at least 95, 96, 97, 98, 99 or 100% sequence identity to SEQ ID NO: 2.

In one embodiment, the multispecific antigen binding protein (AVC-003) comprises: a) a first heavy chain comprising an amino acid sequence with at least 95, 96, 97, 98, 99 or 100% sequence identity to SEQ ID NO: 14; b) a second heavy chain comprising an amino acid sequence with at least 95, 96, 97, 98, 99 or 100% sequence identity to SEQ ID NO: 12; and c) a first light chain and a second light chain comprising an amino acid sequence with at least 95, 96, 97, 98, 99 or 100% sequence identity to SEQ ID NO: 2.

In one embodiment, the multispecific antigen binding protein (AVC-004) comprises: a) a first heavy chain comprising an amino acid sequence with at least 95, 96, 97, 98, 99 or 100% sequence identity to SEQ ID NO: 11; b) a second heavy chain comprising an amino acid sequence with at least 95, 96, 97, 98, 99 or 100% sequence identity to SEQ ID NO: 15; and c) a first light chain and a second light chain comprising an amino acid sequence with at least 95, 96, 97, 98, 99 or 100% sequence identity to SEQ ID NO: 2.

In one embodiment, the multispecific antigen binding protein (AVC-007) comprises: a) a first and second heavy chain comprising an amino acid sequence with at least 95, 96, 97, 98, 99 or 100% sequence identity to SEQ ID NO: 18; and b) a first light chain and a second light chain comprising an amino acid sequence with at least 95, 96, 97, 98, 99 or 100% sequence identity to SEQ ID NO: 21.

In one embodiment, the multispecific antigen binding protein (AVC-008) comprises: a) a first and second heavy chain comprising an amino acid sequence with at least 95, 96, 97, 98, 99 or 100% sequence identity to SEQ ID NO: 19; and b) a first light chain and a second light chain comprising an amino acid sequence with at least 95, 96, 97, 98, 99 or 100% sequence identity to SEQ ID NO: 2.

### Biological Activities of the Multispecific Antigen Binding Protein

A multispecific antigen binding protein as described herein can have a one or more biological activities, including e.g. antigen (TAA) binding, binding to an NK cell, the ability to direct an NK cell to a target cell expressing the TAA, activating an NK cell, including inducing hyper-functionality of the NK cell, and/or the ability to elicit lysis of target cell by the (activated/hyper-functional) NK cell.

In one embodiment, a multispecific antigen binding protein as described herein cause an increase in at least one NK cell activity selected from CD107a degranulation, CD107 or CD69 expression, IFNy production, NK cell proliferation and NK cell cytotoxicity, whereby preferably, the increase is at least a factor 0.1, 0.2, 0.5, 1.0, 2.0, 5.0, 10, 20 50 100, 110, 120, 150, 200, 210, 220, 250 or 300 higher as compared to the increase achieved with the same effector:target cell ratio, with the same NK cells and target cells that are not brought into contact with the multispecific antigen binding protein.

In one embodiment, a multispecific antigen binding protein as described herein cause an increase in at least one NK cell activity selected from CD107a degranulation, CD107 or CD69 expression, IFNy production, NK cell proliferation and NK cell cytotoxicity, whereby preferably, the increase is at least a factor 0.1, 0.2, 0.5, 1.0, 2.0, 5.0, 10, 20, 50, 100, 110, 120, 150, 200, 210, 220, 250 or 300 higher as compared to the increase achieved with the same effector:target cell ratio, with the same NK cells and target cells that are brought into contact (under otherwise identical conditions) with a reference antigen binding protein.

In one embodiment, the reference antigen binding protein is a conventional human IgG1 monoclonal antibody that binds to the same TAA, preferably, that binds to the same epitope, more preferably that has same TAA-specific antigen-binding region(s) as the multispecific antigen binding protein. For example, compared to the monoclonal antibody trastuzumab, a multispecific antigen binding protein having a HER2-binding region (preferably having the trastuzumab variable domains), is superior in causing an increase in NK cell activities.

In one embodiment, the reference antigen binding protein is a (multispecific) antigen binding protein comprising at least one antigen-binding region that binds to the same TAA, preferably, that binds to the same epitope, more preferably that has same TAA-specific antigen-binding region(s) as the multispecific antigen binding protein, and comprising at least one antigen-binding region that specifically binds an NK cell activating receptor such as NKp46, NKp44, NKp30, NKG2D, DNAM1 and CD16A. In one embodiment, the reference antigen binding protein is a (multispecific) antigen binding protein comprising at least one antigen-binding region that binds to the same TAA, preferably, that binds to the same epitope, more preferably that has same TAA-specific antigen-binding region(s) as the multispecific antigen binding protein, and comprising at least one NK cell-activating cytokine other than an IL21R agonist, preferably, at least one NK cell-activating cytokine other than an IL21R agonist and a 4-1 BB agonist. The NK cell-activating cytokine other than at least one of an IL21R agonist and a 4-1BB agonist, can be an IL-15 receptor agonist, such as IL15, for example a human modified IL-15 cross-linker as described in US2018282386 and Vallera et al. (2016, Clin Cancer Res.; 22(14): 3440-3450).

In one embodiment, the reference antigen binding protein is an NK cell engager, such as e.g. described in WO2016/207278, WO 2018/148445, WO2018/152518, WO2019195409 US2018282386, Vallera et al. (2016, supra) and Demaria et al. (2021, supra). One example of a (multispecific) reference antigen binding protein is for example AVC-006 described in the Examples herein, comprising one HER2-binding region and one NKG2D-binding region.

Assays which detect the expression of an NK activation marker or which detects NK cell cytotoxicity, or for detecting NK cell activation and cytotoxicity assays (e.g. short and long term cytotoxicity assays) are described in the Examples herein, as well as for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; EI-Sherbiny et al, (2007) Cancer Research 67(18):8444-9; Nolte-'t Hoen et al, (2007) Blood 109:670-673); WO 2016/207278 and WO 2018/148445.

In one embodiment, a multispecific antigen binding protein as described herein has the ability to induce hyper-functionality (or a hyper-functional phenotype) in an NK cell or in a population of NK cells. A hyper-functional NK cell phenotype is herein understood as a phenotype that has one or more of the features of the phenotype that is obtained by expanding NK cells obtained from donors ex vivo by co-culturing them with irradiated K562 feeder cells modified to express membrane bound IL-21 (mbIL-21) and 4-1BB ligand (FC21 feeder cells) as described by Denman et al. (2012, supra). Thus, in one embodiment, ex vivo expansion of donor NK cells by co-culturing (e.g. for 7, 14 or 21 days), with a multispecific antigen binding protein as described herein, produces a population of NK cells having one or more (or preferably all) of the features selected from the group: a) the fold expansion of the expanded NK cells is at least 0.001, 0.002, 0.005, 0.01, 0.02, 0.05, 0.1, 0.2, 0.5, 1.0, 2.0 or 5.0 fold of the fold expansion of expanded NK cells obtained by ex vivo expansion by co-culturing with irradiated FC21 feeder cells; b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0 or 5.0 fold of the percentage telomere length increase of expanded NK cells obtained by ex vivo expansion by co-culturing with irradiated FC21 feeder cells; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0 or 5.0 fold of the expression level on NK cells obtained upon ex vivo expansion in the presence of FC21 feeder cells; d) the secretion of at least one cytokine of TNF-α, IFN-γ and IL-6 by the expanded NK cells is at least 0.1, 0.2, 0.5, 1.0, 2.0 or 5.0 fold of the secretion of the cytokine by NK cells obtained upon ex vivo expansion in the presence of FC21 feeder cells; and, e) the cytotoxicity of the expanded NK cells is at least 0.001, 0.002, 0.005, 0.01, 0.02, 0.05, 0.1, 0.2, 0.5, 1.0, 2.0 or 5.0 fold of the cytotoxicity of NK cells obtained upon ex vivo expansion in the presence of FC21 feeder cells. In a preferred embodiment, the ex vivo expansion of donor NK cells further comprises that the NK cells are co-cultured with tumor cells expressing a TAA specifically bound by the multispecific antigen binding protein. Protocols for ex vivo expansion of donor NK cells and assays for determining fold expansion, telomere length increase, expression level of NK cell activating receptors, cytokine secretion and cytotoxicity (e.g. short term or long term cytotoxicity assays) are described in Denman et al. (2012, supra) and in the Examples herein.

### Pharmaceutical Compositions

In a further aspect, the invention relates to a pharmaceutical composition comprising a multispecific antigen binding protein as described herein, and a pharmaceutically acceptable carrier (excipient). The pharmaceutically acceptable carrier such as an adjuvant, or vehicle, is for administration of the polypeptide to a subject. Said pharmaceutical composition can be used in the methods of treatment described herein below by administration of an effective amount of the composition to a subject in need thereof. The term “subject”, as used herein, refers to all animals classified as mammals and includes, but is not restricted to, primates and humans. The subject is preferably a male or female human of any age or race.

The term “pharmaceutically acceptable carrier”, as used herein, is intended to include any and all solvents, dispersion media, coatings, antibacterial and antifungal agents, isotonic and absorption delaying agents, and the like, compatible with pharmaceutical administration (see e.g. “Handbook of Pharmaceutical Excipients”, Rowe et al eds. 7^{th} edition, 2012, www.pharmpress.com). The use of such media and agents for pharmaceutically active substances is well known in the art. Except insofar as any conventional media or agent is incompatible with the active compound, use thereof in the compositions is contemplated. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter ions such as sodium; metal complexes (e.g. Zn²⁺-protein complexes); and/or non-ionic surfactants such as TWEEN™, PLURONICS™ or polyethylene glycol (PEG).

Supplementary active compounds can also be incorporated into the pharmaceutical composition of the invention. Thus, in a particular embodiment, the pharmaceutical composition of the invention may also contain more than one active compound as necessary for the particular indication being treated, preferably those with complementary activities that do not adversely affect each other. For example, it may be desirable to further provide a chemotherapeutic agent, a cytokine, an analgesic agent, a thrombolytic or an immunomodulating agent, e.g. an immunosuppressive agent or an immunostimulating agent. The effective amount of such other active agents depends, among other things, on the amount of the polypeptide of the invention present in the pharmaceutical composition, the type of disease or disorder or treatment, etc.

In one embodiment, the polypeptide of the invention is prepared with carriers that will protect said compound against rapid elimination from the body, such as a controlled release formulation, including implants and microencapsulated delivery systems, e.g. liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions, including targeted liposomes can also be used as pharmaceutically acceptable carriers. These can be prepared according to methods known to those skilled in the art, for example, as described in U.S. Pat. No. 4,522,811 or WO2010/095940.

The administration route of the polypeptide of the invention can be parenteral. The term “parenteral” as used herein includes intravenous, intra-arterial, intralymphatic, intraperitoneal, intramuscular or subcutaneous. The intravenous or intramuscular forms of parenteral administration are preferred. By “systemic administration” is meant intravenous, intraperitoneal and intramuscular administration. The amount of the polypeptide required for therapeutic or prophylactic effect will, of course, vary with the polypeptide chosen, the nature and severity of the condition being treated and the patient. In addition, the polypeptide may suitably be administered by pulse infusion, e.g., with declining doses of the polypeptide. Preferably the dosing is given by injections, most preferably intravenous, intramuscular or subcutaneous injections, depending in part on whether the administration is brief or chronic.

Thus, in a particular embodiment, the pharmaceutical composition of the invention may be in a form suitable for parenteral administration, such as sterile solutions, suspensions or lyophilized products in the appropriate unit dosage form. Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CremophorEM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition.

Prolonged absorption of the injectable compositions can be brought about by including in the composition an agent which delays absorption, for example, aluminium monostearate and gelatin.

Sterile injectable solutions can be prepared by incorporating the active compound (e.g a polypeptide of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In a particular embodiment, said pharmaceutical composition is administered via intravenous (IV), intramuscular (IM) or subcutaneous (SC) route. Adequate excipients can be used, such as bulking agents, buffering agents or surfactants. The mentioned formulations will be prepared using standard methods for preparing parenterally administrable compositions as are well known in the art and described in more detail in various sources, including, for example, “Remington: The Science and Practice of Pharmacy” (Ed. Allen, L. V. 22nd edition, 2012, www.pharmpress.com).

It is especially advantageous to formulate the pharmaceutical compositions, namely parenteral compositions, in dosage unit form for ease administration and uniformity of dosage. Dosage unit form as used herein refers to physically discrete units suited as unitary dosages for the subject to be treated; each unit containing a predetermined quantity of active compound (polypeptide of the invention) calculated to produce the desired therapeutic effect in association with the required pharmaceutical carrier. The specification for the dosage unit forms of the invention is dictated by and directly dependent on the unique characteristics of the active compound and the particular therapeutic effect to be achieved, and the limitations inherent in the art of compounding such an active compound for the treatment of individuals.

Generally, for the prevention and/or treatment of the diseases and disorders mentioned herein and depending on the specific disease or condition to be treated and its severity, the potency of the specific polypeptide of the invention to be used, the specific route of administration and the specific pharmaceutical formulation or composition used, the polypeptide of the invention will generally be administered in the range of from 0.001 to 1,000 mg/kg body weight/day, preferably about 0.01 to about 100 mg/kg body weight/day, most preferably from about 0.05 to 10 mg/kg body weight/day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. The pharmaceutical compositions can be included in a container, pack, or dispenser together with instructions for administration.

### Therapeutic Use

In another aspect there is provided a multispecific antigen binding protein as described herein for use as a medicament. In one embodiment, the multispecific antigen binding is used as protein as described herein is used as an active ingredient, component or substance in a medicament.

In one aspect, the invention pertains to a use of a multispecific antigen binding protein as described herein for the manufacture of a medicament, e.g. a pharmaceutical preparation comprising the multispecific antigen binding protein as an active ingredient, for the treatment, prevention or diagnosis of a disease in a subject in need thereof.

In one aspect, the invention pertains to a multispecific antigen binding protein as described herein, or a pharmaceutical preparation comprising the multispecific antigen binding protein as an active ingredient, for use in the treatment, prevention or diagnosis of a disease in a subject in need thereof.

In one aspect, the invention pertains to a method for the treatment of a disease in a subject in need thereof, wherein the method comprises the step of administering to the subject (an effective amount of) a multispecific antigen binding protein as described herein, or a pharmaceutical preparation comprising the multispecific antigen binding protein as an active ingredient.

The disease to be treated, prevented or diagnosed using the multispecific antigen binding protein can be a cancer, an infectious disease, an inflammatory disease or an autoimmune disease.

In one embodiment, the disease to be treated, prevented or diagnosed using the multispecific antigen binding protein is a cancer, e.g. a cancer as described below. The cancer preferably is a cancer expressing a TAA as described herein above.

In one embodiment, the treatment can comprise the steps of a) identifying a TAA expressed by (tumor) cells in the cancer; b) selection of multispecific antigen binding protein as described herein that specifically binds the TAA; c) using the multispecific antigen binding protein selected in b) in the treatment of the cancer. The cancer can be a cancer as described below.

In one embodiment, the invention pertains to a method for enhancing anti-tumor activity of an NK cell in a subject, the method comprising the step of administering to the subject a multispecific antigen binding protein as described herein, or a pharmaceutical preparation comprising the multispecific antigen binding protein as an active ingredient. In one embodiment, the subject has cancer, e.g. a cancer as described below. Preferably, the cancer comprising tumor cells expressing the TAA.

In one embodiment, the invention pertains to a method for expanding and/or inducing hyper-functionality NK cells a in a subject, the method comprising the step of administering to the subject a multispecific antigen binding protein as described herein, or a pharmaceutical preparation comprising the multispecific antigen binding protein as an active ingredient. The fold expansion and the hyper-functionality preferably is as herein described above. In one embodiment, the subject has cancer, preferably a cancer comprising tumor cells expressing the TAA.

Subjects having cancer, often present with lower numbers of NK cells and/or with exhausted NK cells. The multispecific antigen binding proteins of the invention can thus be advantageously used to expand the numbers of NK cells and/or to induce hyper-functionality of the NK cells in a subject suffering from cancer. A further advantage of the hyper-functionality of NK cells as induced by the multispecific antigen binding protein of the invention, includes their increased secretion of cytokines such as TNF-α, IFN-γ and IL-6, which help shape adaptive immune response involving DCs and T cells. Indeed, NK cells have been reported to promote the recruitment to the tumor micro environment of a DC subset specializing in the crosspresentation of tumor antigens to CD8⁺ T cells, suggesting a crucial role for NK cells in the potentiation of antitumor CD8⁺ T cell responses (Bottcher et al., Cell, 2018. 172: 1022-1037; and Barry et al., Nat. Med. 2018. 24: 1178-1191). The contribution of NK cells to the orchestration of antitumor T-cell responses has also been confirmed experimentally in mice, demonstrating that, in addition to their direct effector functions, NK cell can promote T-cell responses and long-lasting immune control of tumors (Bonavita et al., Immunity 2020. 53: 1215-1229). In one embodiment, the TAA is a TAA as defined herein above and/or an antigen expressed on the surface of a malignant cell of a type of cancer as described below. A subject to be treated in accordance with the methods of the invention can have a cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sézary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukemia (T-Lbly/T-ALL).

In one embodiment, a multispecific antigen binding protein as described herein can be used as a monotherapy (i.e. without other therapeutic agents). In another embodiment, a multispecific antigen binding protein as described herein can be used in combined treatments.

In one embodiment, a multispecific antigen binding protein as described herein is used in combination with another immunotherapy, e.g. a cellular immunotherapy. The multispecific antigen binding protein can thus be used in combination with the adoptive transfer of immune cells, including the adoptive transfer of T cells, e.g. CAR T cells, or NK cells. The NK cells can e.g. be enriched or expanded by methods known in the art or can be ex vivo NK cells as herein described.

In one embodiment, a multispecific antigen binding protein as described herein can be used in combined treatments with one or more other therapeutic agents. The additional therapeutic agent or agents normally utilized for the particular therapeutic purpose for which an antibody against a TAA is being administered. The additional therapeutic agent or agents will normally be administered in amounts and treatment regimens typically used for that agent in a monotherapy for the particular disease or condition being treated. Such therapeutic agents when used in the treatment of cancer, include, but are not limited to anti-cancer agents and chemotherapeutic agents. Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and luteinizing hormone releasing factor.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PD-L1, (iv) LAG3, (v) B7-H3, (vi) B7-H4, and (vii) TIM3. Yet other agents that may be used as part of a combination therapy in treating cancer are monoclonal antibodies against TAA as described herein above.

In some embodiments the administration of the multispecific antigen binding protein and the other therapeutic agent can elicit an additive or synergistic effect on immunity and/or on therapeutic efficacy.

In one embodiment, a multispecific antigen binding protein as described herein is used as at least one of an neoadjuvant therapy and an adjuvant therapy, in addition to a primary therapy comprising e.g. surgery and/or radiation therapy. As a neoadjuvant therapy, the multispecific antigen binding protein is administered before the primary treatment, e.g. to help reduce the size of a tumor (such that less extensive surgery and/or radiation therapy is required), kill cancer cells that have spread (e.g. micrometastatic disease) and/or reduce the risk of tumor cells spreading post-surgery. As an adjuvant therapy, the multispecific antigen binding protein is administered after the primary treatment, e.g. to treat minimal residual disease (destroy remaining cancer cells). The use of the multispecific antigen binding protein as an neoadjuvant therapy and/or an adjuvant therapy lowers relapse rates. In the neoadjuvant therapy and/or adjuvant therapy, the multispecific antigen binding protein can be used as monotherapy or in in combined treatments as described above.

### Ex Vivo Methods

In a further aspect, the invention relates to methods wherein a multispecific antigen binding protein as described herein is used for ex vivo (in vitro) treatment of an NK cell or a population of NK cells. The method can be a method for at least one of expanding, preactivating, activating, enhancing cytotoxicity and/or cytokine production, and inducing a hyper-functional phenotype as defined above. The methods at least comprise the step of contacting an NK cell or a population thereof, with a multispecific antigen binding protein as described herein or with a composition comprising the multispecific antigen binding protein. In a preferred embodiment, the method comprises the further step of co-culturing the NK cells with tumor cells expressing a TAA specifically bound by the multispecific antigen binding protein. Preferably, the NK cells are co-cultured with the tumor cells expressing the TAA specifically bound by the multispecific antigen binding protein, in the presence of the multispecific antigen binding protein.

An NK cell or a population of NK cells for ex vivo treatment can be enriched from peripheral blood mononuclear cells (PBMCs). Methods for enrichment and ex vivo treatment of NK cells from PBMCs are e.g. described in Denman et al. (pLoS One. 2012;7(1):e30264) and in US2020/0061115. For example, NK cells enriched from PBMCs can be seeded at 0.1×10⁶ NK cells/mL in SCGM (CellGenix, Portsmouth, N.H.), supplemented with 10% FBS, 2 mM Glutamax, 100 U/mL IL-2 (Peprotech, Rocky Hill, N.J.) and 1, 2, 5, 10, 20, 50, 100, 200, 500, 1000 μg/mL of one or more multispecific antigen binding proteins as described herein. Media with supplements can be refreshed every 2-3 days.

It is understood that the duration of the contact between the NK cells and a multispecific antigen binding protein as described herein (i.e. the duration of the preactivation or activation (i.e., the duration of expanding, preactivating, activating, enhancing cytotoxicity and/or cytokine production and inducing a hyper-functional phenotype) can be for any length of time necessary to achieve the desired phenotype of the NK cells. For example, the contact can be as little as 1 minute or as much as 7 days (for example, culturing the NK cells in the presence of a multispecific antigen binding protein as described herein for 7 days). In one embodiment of the method, the NK cells are contacted with the multispecific antigen binding protein for 5, 6, 7, 8, 9, 10, 11, 12, 13, 14, 15, 16, 17, 18, 19, 20, 21, 22, 23, 24, 36, or 48 hours. In one embodiment of the method, the NK cells are contacted with the multispecific antigen binding protein for 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 days.

In one embodiment, the ex vivo treated (expanded) NK cells have one or more features selected from: a) the fold expansion of the expanded NK cells is at least 0.001, 0.002, 0.005, 0.01, 0.02, 0.05, 0.1, 0.2, 0.5, 1.0, 2.0 or 5.0 fold of the fold expansion of expanded NK cells obtained by ex vivo expansion by co-culturing with irradiated FC21 feeder cells; b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0 or 5.0 fold of the percentage telomere length increase of expanded NK cells obtained by ex vivo expansion by co-culturing with irradiated FC21 feeder cells; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0 or 5.0 fold of the expression level on NK cells obtained upon ex vivo expansion in the presence of FC21 feeder cells; d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0 or 5.0 fold of the secretion of the cytokine by NK cells obtained upon ex vivo expansion in the presence of FC21 feeder cells; and, e) the cytotoxicity of the expanded NK cells is at least 0.001, 0.002, 0.005, 0.01, 0.02, 0.05, 0.1, 0.2, 0.5, 1.0, 2.0 or 5.0 fold of the cytotoxicity of NK cells obtained upon ex vivo expansion in the presence of FC21 feeder cells.

In yet a further aspect, the invention pertains to a method for the treatment of a disease in a subject in need thereof, wherein the method comprises the step of administering to the subject (an effective amount of) NK cells obtained in the above method for ex vivo treatment of an NK cell or a population of NK cells. Once a sufficient number of NK cells with the desired (hyper-functional) phenotype has been expanded by the ex vivo treatment, the NK cells can be administered to a subject in need thereof.

In one embodiment, the method for the treatment comprises the administration of the ex vivo treated NK cells in combination with a multispecific antigen binding protein as described herein, or a pharmaceutical preparation comprising the multispecific antigen binding protein as an active ingredient.

In one embodiment, the method for the treatment comprises the administration of the ex vivo treated NK cells in combination with another NK cell engager, such as e.g. described in WO2016/207278, WO 2018/148445, WO2018/152518, WO2019195409 US2018282386, Vallera et al. (2016, supra) and Demaria et al. (2021, supra), or with a multispecific antigen binding protein as described in the co-pending application by the same applicant with reference no. P6111865EP. One example of another NK cell engager is for example AVC-006 described in the Examples herein, comprising one HER2-binding region and one NKG2D-binding region. In a further embodiment, the ex vivo treated NK cells can be used in combination with the other engager and with a multispecific antigen binding protein as described herein.

The disease to be treated can be a cancer, an infectious disease, an inflammatory disease or an autoimmune disease, as described above. Preferably, the disease to be treated is a cancer, as described above. The cancer preferably is a cancer expressing a TAA that is specifically bound by the multispecific antigen binding protein that is administered in combination with the ex vivo treated NK cells. The administration of the ex vivo treated NK cells in combination with the multispecific antigen binding protein will facilitate targeting of the administered ex vivo treated NK cells to tumor cells expressing the TAA.

In one embodiment, the ex vivo treated NK are autologous to the subject. In another embodiment, the ex vivo treated NK are allogeneic, e.g. derived from donor PBMCs.

### Nucleic Acids, Host Cells and Methods for Producing Multispecific Antigen Binding Protein

In one aspect, the invention relates to a nucleic acid molecule comprising one or more nucleotide sequences encoding a polypeptide chain of a multispecific antigen binding protein as described herein. The nucleotide sequence encoding such a polypeptide chain preferably encodes a signal peptide operably linked to the polypeptide chain. A nucleic acid molecule comprising one or more of the nucleotide sequences encoding a polypeptide chain, further preferably comprises regulatory elements for (or conducive to) the expression of the polypeptide chain in an appropriate host cell, which regulatory elements are operably linked to the nucleotide sequence.

In one aspect, the invention relates to a host cell comprising the nucleic acid molecule comprising one or more nucleotide sequences encoding a polypeptide chain of a multispecific antigen binding protein as described herein. In one embodiment, the host cell is an isolated cell or a cultured cell. Among the host cells that may be employed are prokaryotes, yeast or higher eukaryotic cells. Prokaryotes include gram-negative or gram-positive organisms, for example Escherichia coli or bacilli. Suitable yeast cells include Saccharomyces cerevisiae and Pichia pastoris. Higher eukaryotic cells include insect cells and established cell lines of mammalian origin. Examples of suitable mammalian host cell lines include the COS-1, COS-7 line of monkey kidney cells (Gluzman et al., 1981, Cell 23:175), L cells, HEK 293 cells, C127 cells, 3T3 cells, Chinese hamster ovary (CHO) cells, HeLa cells, BHK cell lines, e.g. BHK21, BSC-1, Hep G2, 653, SP2/0, and the CVI/EBNA cell line derived from the African green monkey kidney cell line CVI as described by McMahan et al. (1991, EMBO J. 10: 2821). The host cell may be any suitable species or organism capable of producing N-linked glycosylated polypeptides, e.g. a mammalian host cell capable of producing human or rodent IgG type N-linked glycosylation. Appropriate cloning and expression vectors for use with bacterial, fungal, yeast, and mammalian cellular hosts are described by Pouwels et al. (Cloning Vectors: A Laboratory Manual, Elsevier, N.Y., 1985). Host cells comprising the nucleic acid molecule of the invention can be cultured under conditions that promote expression of the polypeptide.

Thus, another aspect the invention relates to a method for producing a multispecific antigen binding protein as described herein. The method preferably comprises culturing a host cell as described above such that one or more nucleotide sequences are expressed and the multispecific antigen binding protein is produced. The method preferably comprises the step of cultivating a host cell comprising one or more of the nucleotide sequences encoding a polypeptide chain of the multispecific antigen binding protein. The host cell is preferably cultured under conditions conducive to expression of the one or more polypeptide chains. The method can further comprise the step of recovering the multispecific antigen binding protein. The multispecific antigen binding protein can be recovered by conventional protein purification procedures, including e.g. protein A-Sepharose, hydroxylapatite chromatography, gel electrophoresis, dialysis, size exclusion chromatograpy or affinity chromatography, using e.g. streptavidin/biotin (see e.g. Low et al., 2007, J. Chromatography B, 848:48-63; Shukla et al., 2007, J. Chromatography B, 848:28-39).

In a further aspect, the invention relates to a method for producing a pharmaceutical composition comprising a multispecific antigen binding protein as described herein, the method comprising the steps of a) producing the multispecific antigen binding protein in a method as defined above; and b) formulating the multispecific antigen binding protein with a pharmaceutically acceptable carrier as defined above, to obtain a pharmaceutical composition.

The present invention has been described above with reference to a number of exemplary embodiments as shown in the drawings and examples. Modifications and alternative implementations of some parts or elements are possible and are included in the scope of protection as defined in the appended claims.

### DESCRIPTION OF THE FIGURES

FIG. 1. Schematic depiction of a multispecific antigen binding protein as described herein that engages an NK cell (lower plasma membrane) with surface expression of 4-1 BB, an activating Fc receptor (FcγRIIIa), IL21R and an activating stress ligand receptor, to a tumor cell (upper plasma membrane) with surface expression of tumor-associated antigens (TAA) and a stress ligand.

FIG. 2. Schematic depiction of trastuzumab-based multispecific antigen binding protein and control proteins AVC-001 to AVC-008 (see Example 1 for sequence details). AVC-001 to AVC-006 are heterodimeric. AVC-007 and AVC-008 are homodimeric. Tras=trastuzumab; 41 BBLT=4-1 BB ligand trimer; 41 BBLM=4-1 BB ligand monomer.

FIG. 3. Cytotoxic activity against SKOV-3 HER2-positive tumor cells, over time (in hours) of purified NK cells in the absence (NK only) or in the presence of the multispecific antigen binding proteins AVC-001 or AVC-007 (trastuzumab analogs containing both 4-1 BBL and IL-21 cytokines), or the control proteins AVC-003 (trastuzumab analog with only IL-21) or AVC-005 (trastuzumab analog without cytokines). Percentage of cell lysis was determined as described in Example 3.3.1.

FIG. 4. Proliferation of NK cells as determined by dilution of CellTrace Violet by flow cytometry. Each peak represents a division. AVC1 and AVC7 are NK cells induced to proliferate by multispecific antigen binding proteins AVC-001 and AVC-007, resp.. Trast. are NK cells induced to proliferate by the Trastuzumab analog AVC-005 as control protein. Vehicle control is a co-culture of NK cells with target cells without the addition of stimulatory proteins.

FIG. 5. Proliferation of NK cells co-cultured with SKOV-3 tumor cells in the presence or absence of multispecific antigen binding proteins. Proliferation was measured using CellTrace Violet dilution. The division index is the average number of divisions that a cell in the original population has undergone, including the cells which never divided. A) Dose-responses for proliferation of NK cells induced by the presence of multispecific antigen binding proteins AVC-001 (AVC1) or AVC-007 (AVC7) at indicated concentrations, compared to that of the AVC-005 Trastuzumab analog control protein (Trast.). Vehicle is co-culture of SKOV-3 tumor cells and NK cells without protein addition. B) A comparison of the proliferation of NK cells induced by co-culture with SKOV-3 tumor cells in the presence of 1.6 nM of the multispecific antigen binding proteins AVC-001 (AVC1-IL-21 and 4-1 BBL), AVC-003 (AVC3-IL-21), AVC-004 (AVC4-4-1 BBL) or the AVC-005 Trastuzumab analog control protein (AVC5) or in the absence of protein addition (NK+SKOV3).

FIG. 6. Prolonged ex vivo expansion of NK cells stimulated by multispecific antigen binding proteins AVC-001 (AVC1) and AVC-007 (AVC7), compared to that of NK cells stimulated by irradiated FC21 feeder cells (FC21), the AVC-005 Trastuzumab analog control protein (Trast.) and NK cells without stimulation (NK cells only).

FIG. 7. Shown here is a viSNE analysis as described in Example 4.3, using 31 markers, which shows the population distribution of cells from different expansion methods. AVC1, AVC7 and FC21 are after 3 weeks of expansion as described in Example 3.5, in the presence of the multispecific antigen binding proteins AVC-001 or AVC-007, or FC21 feeder cells, resp.. The color mapping indicates CD56 expression, which is used to identify NK cells. Compared to the control condition of unexpanded NK cells, all 3 expansion methods result in an enrichment of highly CD56-expressing NK cells which have a very similar phenotype. The four panels 232, 233, 234 and 235 per expansion method represent NK cells from four different donors.

FIG. 8. Up- or down regulation of NK cell-surface markers as indicated after 3 weeks of expansion of NK cells as described in Example 3.5, in the presence of the multispecific antigen binding proteins AVC-001 (AVC1) or AVC-007 (AVC7), or FC21 feeder cells (FC21), resp.. Fold change (Log2) is compared to the control condition of unexpanded NK cells.

FIG. 9. Enhanced cytotoxicity towards MDA-MB-231 HER2-positive tumor cells of NK cells stimulated to expand and activate by multispecific antigen binding proteins AVC-001 (AVC1) and AVC-007 (AVC7). NK cells that were prestimulated for 1 week and up to 3 weeks with AVC-001 or AVC-007 show increased functionality against tumor cells as compared to timepoint 0 (unstimulated NK cells). Cytotoxicity was determined by Calcein AM release.

FIG. 10. Increased interferon-gamma production by NK cells co-cultured with SKOV-3 HER2-positive tumor cells, after stimulation of with multispecific antigen binding proteins AVC-001 (AVC1) and AVC-007 (AVC7).

FIG. 11. Long-term cytotoxicity against SKOV-3 tumor cells by NK cells stimulated with multispecific antigen binding protein AVC-001 or AVC-007 upon repeated co-culture with SKOV-3 tumor cells. After each 3-day cycle, NK cells were were harvested and used to set up a new cycle of co-culture with fresh target cells at a 1:1 E:T ratio. The multispecific antigen binding proteins AVC-001 (AVC1) orAVC-007 (AVC7) or the AVC-005 control protein (AVC5=trastuzumab analog) were added at the start of each 3-day cycle to expose NK cells to new booster proteins at the beginning of every cycle. Control wells contained only NK cells and SKOV-3 target cells (NK+SKOV3), or only SKOV-3 target cells (SKOV3 only).

FIG. 12. Long-term NK cell cytotoxicity induced by the multispecific antigen binding proteins AVC-001 and AVC-016 to AVC-021, against tumor cell lines expressing the respective tumor-associated antigens: (A) AVC-001 (AVC1) with SKOV-3 tumor cells expressing HER2; (B) AVC-016 (AVC16) with BxPC3 tumor cells expressing TROP2; (C) AVC-017 (AVC17) with U87MG tumor cells expressing GD2; (D) AVC-018 (AVC18) with SKOV-3 tumor cells expressing FOLR1; (E) AVC-019 (AVC19) with U87MG tumor cells expressing B7-H3, (F) AVC-020 (AVC20) with A431 tumor cells expressing EGFR; and, (G) AVC-21 (AVC21) with MDA-MB-231 tumor cells expressing PD-L1. NK cell cytotoxicity induced by the multispecific antigen binding proteins against tumor cells that express the respective antigens (open squares) is followed over time (hours) and compared to the cytotoxicity of NK cells only (solid circles).

FIG. 13. Interferon-gamma levels after co-culture with tumor cells that express the respective antigen. Data is shown as fold change over vehicle (NK cells +tumor cells). (A) AVC-001 (AVC1) with SKOV-3 tumor cells expressing HER2; (B) AVC-016 (AVC16) with BxPC3 tumor cells expressing TROP2; (C) AVC-017 (AVC17) with U87MG tumor cells expressing GD2; (D) AVC-018 (AVC18) with SKOV-3 tumor cells expressing FOLR1; (E) AVC-019 (AVC19) with U87MG tumor cells expressing B7-H3, (F) AVC-020 (AVC20) with A431 tumor cells expressing EGFR; and, (G) AVC-21 (AVC21) with MDA-MB-231 tumor cells expressing PD-L1.

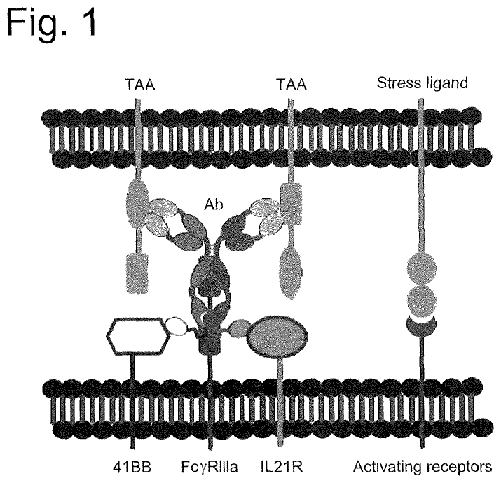

### EXAMPLES

### Example 1 Preparation of Multispecific Antigen Binding Proteins

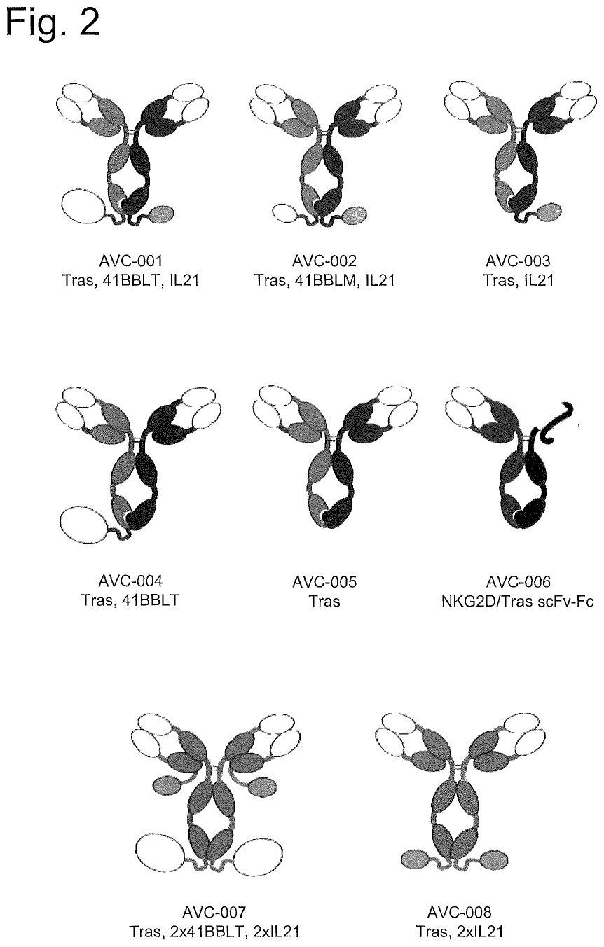

Coding sequences were generated for the multispecific antigen binding proteins and control proteins, including proteins AVC-001 to AVC-008 as schematically depicted in FIG. 2, the amino acid sequences of which are depicted in Tables 1.1.1 and 1.1.2.

Expression constructs for expression of the coding sequences in the HEK293-E-253 cell line were prepared using standard molecular biological materials and techniques. Briefly, coding sequences were generated by direct synthesis and/or by PCR. PCR was performed using the PrimeSTAR MAX DNA polymerase (Takara, #R045A) and PCR products were purified from 1% agarose gel using the NucleoSpin gel and PCR clean-up kit (Macherey-Nagel, #740609.250). Once purified, the PCR products were quantified prior to the In-Fusion ligation reaction which was performed as described in the manufacturer's protocol (ClonTech, #ST0345). The plasmids were obtained after a miniprep preparation. Plasmids were then sequenced for sequence confirmation before being transfected into the HEK293E-253 cell line.

All used buffers were made using Versylene (endotoxin-free and sterile) water. Endotoxin was removed from instruments by incubation with 0.1 M NaOH for at least 16 hours. Line clearance was performed at start and end of the production.

HEK293E-253 cells were transfected with endotoxin-free plasmid DNA using the rPEx® technology. Six days post transfection conditioned medium containing recombinant protein was harvested by centrifugation and filtration over a 0.22 μm bottletop filter. A 100 μl sample was stored at 4° C. To avoid binding of a-specific proteins or cell fragments to the MabSelect PrismA resin, the sample was conditioned with 40 ml, 5 M NaCl per 1 L medium. This results in an increase of the NaCl concentration with 0.2 M.

A HiScreen Fibro PrismA column was equilibrated in 20 mM Tris 150 mM NaCl pH 7.8. The recombinant protein in the conditioned medium was loaded onto the column using a Teledyne ASX-560 autosampler and a-specific bound proteins were removed by washing the column with 20 mM Tris 1 M NaCl pH 7.8 and with 20 mM citrate 150 mM NaCl pH 5.0. The bound recombinant protein was eluted by a 12 CV block gradient to 20 mM citrate 150 mM NaCl pH 3.5 and a 6 CV block gradient to 20 mM citrate 150 mM NaCl pH 3.0. The eluate was directly in-line neutralized by mixing-in of 1 M Tris pH 8.0 for 70ot hat70zationn to pH 7 in a 1.0/0.2 ratio and 12.5 ml fractions were collected. Between injections, the HiScreen Fibro PrismA column was regenerated using 5 CV 0.5 M NaOH, 2 CV 1 M Tris pH 8.0 and equilibrated in 20 mM Tris 150 mM NaCl pH 7.8. The Teledyne autosampler was cleaned with 15 ml 0.1 M NaOH and equilibrated in PBS.

The HiTrap Fibro PrismA pool was concentrated to 2-3 ml per injection on a Superdex200 increase 16/40 column using an Amicon 30 kDa spin filter. Aggregates in the concentrated pool were removed by filtration over a 0.22 μm syringe filter. The concentrated sample was stored at 4° C. before gelfiltration.

The recombinant protein products were analyzed by Labchip capillary electrophoresis and LAL assay.

TABLE 1.1.1
Overview of the multispecific antigen binding proteins AVC-001-AVC-008.

| Construct | Heavy chain 1 | | Heavy chain 2 | | Light chain 1 | | Light chain 2 | |
|---|---|---|---|---|---|---|---|---|
| AVC-001 | Trastuzumab | SEQ ID NO: | Trastuzumab | SEQ ID NO: | Trastuzumab | SEQ ID NO: | Trastuzumab | SEQ ID NO: |
| | HC1-4-1BBL | 11 | HC2-IL21 | 12 | LC | 2 | LC | 2 |
| | trimer fusion | | fusion | | | | | |
| AVC-002 | Trastuzumab | SEQ ID NO: | Trastuzumab | SEQ ID NO: | Trastuzumab | SEQ ID NO: | Trastuzumab | SEQ ID NO: |
| | HC1-4-1BBL | 13 | HC2-IL21 | 12 | LC | 2 | LC | 2 |
| | monomer | | fusion | | | | | |
| | fusion | | | | | | | |
| AVC-003 | Trastuzumab | SEQ ID NO: | Trastuzumab | SEQ ID NO: | Trastuzumab | SEQ ID NO: | Trastuzumab | SEQ ID NO: |
| | HC1 | 14 | HC2-IL21 | 12 | LC | 2 | LC | 2 |
| | | | fusion | | | | | |
| AVC-004 | Trastuzumab | SEQ ID NO: | Trastuzumab | SEQ ID NO: | Trastuzumab | SEQ ID NO: | Trastuzumab | SEQ ID NO: |
| | HC1-4-1BBL | 11 | HC2 | 15 | LC | 2 | LC | 2 |
| | trimer fusion | | | | | | | |
| AVC-005 | Trastuzumab | SEQ ID NO: | Trastuzumab | SEQ ID NO: | Trastuzumab | SEQ ID NO: | Trastuzumab | SEQ ID NO: |
| | HC1 | 14 | HC2 | 15 | LC | 2 | LC | 2 |
| AVC-006 | Anti-NKG2D | SEQ ID NO: | Trastuzumab- | SEQ ID NO: | Anti-NKG2D | SEQ ID NO: | — | — |
| | HC1 | 16 | scFv-HC2 | 17 | LC | 20 | | |
| AVC-007 | Trastuzumab | SEQ ID NO: | Trastuzumab | SEQ ID NO: | Trastuzumab | SEQ ID NO: | Trastuzumab | SEQ ID NO: |
| | HC-4-1BBL | 18 | HC-4-1BBL | 18 | LC-IL21 | 21 | LC-IL21 | 21 |
| | trimer fusion | | trimer fusion | | fusion | | fusion | |
| AVC-008 | Trastuzumab | SEQ ID NO: | Trastuzumab | SEQ ID NO: | Trastuzumab | SEQ ID NO: | Trastuzumab | SEQ ID NO: |
| | HC-IL21 | 19 | HC-IL21 | 19 | LC | 2 | LC | 2 |
| | fusion | | fusion | | | | | |

TABLE 1.1.2
Annotated amino acid sequences of multispecific
binding molecules AVC-001-AVC-005 and AVC-007-AVC-008.
Heavy chain 1 of AVC-001 and AVC-004 (SEQ ID NO: 11) compared with
VH3/3-11 (SEQ ID NO: 22) and a human IgG1 constant region (SEQ ID NO: 23)

| | CDR-H1 CDR-H2 | |
|---|---|---|
| AVC-001 | EVQLVESGGG LVQPGGSLRL SCAASGFNIK DTYIHWVRQA PGKGLEWVAR IYPTNGYTRY ADSVKGRFTI SADTSKNTAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-001 | LQMNSLRAED TAVYYCSRWG GDGFYAMDYW GQGTLVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-001 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-001 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-001 | EYKCKVSNKA LPAPIEKTIS KAKGQPREPQ VYTLPPCREE MTKNQVSLWC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-001 | LDSDGSFFLY SKLTVDKSRW QQGNVFSCSV MHEALHNHYT QKSLSLSPGK GGGGSGGGGS GGGGSREGPE LSPDDPAGLL | 480 |
| HuIgG1 | LDSDGSFFLY SKLTVDKSRW QQGNVFSCSV MHEALHNHYT QKSLSLSPGK | |
| AVC-001 | DLRQGMFAQL VAQNVLLIDG PLSWYSDPGL AGVSLTGGLS YKEDTKELVV AKAGVYYVFF QLELRRVVAG EGSGSVSLAL | 560 |
| | 4-1BBL ECD | |
| AVC-001 | HLQPLRSAAG AAALALTVDL PPASSEARNS AFGFQGRLLH LSAGQRLGVH LHTEARARHA WQLTQGATVL GLFRVTPEIP | 640 |
| | Linker | |
| AVC-001 | AGLPSPRSEG GGGSGGGGSG GGGSGGGGSR EGPELSPDDP AGLLDLRQGM FAQLVAQNVL LIDGPLSWYS DPGLAGVSLT | 720 |
| | 4-1BBL ECD | |
| AVC-001 | GGLSYKEDTK ELVVAKAGVY YVFFQLELRR VVAGEGSGSV SLALHLQPLR SAAGAAALAL TVDLPPASSE ARNSAFGFQG | 800 |
| | Linker | |
| AVC-001 | GLLHLSAGQR LGVHLHTEAR ARHAWQLTQG ATVLGLFRVT PEIPAGLPSP RSEGGGGSGG GGSGGGGSGG GGSREGPELS | 880 |
| | 4-1BBL ECD | |
| AVC-001 | PDDPAGLLDL RQGMFAQLVA QNVLLIDGPL SWYSDPGLAG VSLTGGLSYK EDTKELVVAK AGVYYVFFQL ELRRVVAGEG | 960 |
| AVC-001 | SGSVSLALHL QPLRSAAGAA ALALTVDLPP ASSEARNSAF GFQGRLLHLS AGQRLGVHLH TEARARHAWQ LTQGATVLGL | 1040 |
| AVC-001 | FRVTPEIPAG LPSPRSE | 1057 |

Heavy chain 2 of AVC-001, AVC-002 and AVC-003 (SEQ ID NO: 12) compared with
VH3/3-11 (SEQ ID NO: 22) and a human IgG1 constant region (SEQ ID NO: 23)

| | | |
|---|---|---|
| | CDR-H1 CDR-H2 | |
| AVC-001 | EVQLVESGGG LVQPGGSLRL SCAASGFNIK DTYIHWVRQA PGKGLEWVAR IYPTNGYTRY ADSVKGRFTI SADTSKNTAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-001 | LQMNSLRAED TAVYYCSRWG GDGFYAMDYW GQGTLVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-001 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-001 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-001 | EYKCKVSNKA LPAPIEKTIS KAKGQPREPQ VCTLPPSREE MTKNQVSLSC AVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Knobs-Into-Hole Linker | |
| AVC-001 | LDSDGSFFLV SKLTVDKSRW QQGNVFSCSV MHEALHNHYT QKSLSLSPGK GGGGSQGQDR HMIRMRQLID IVDQLKNYVN | 480 |
| HuIgG1 | LDSDGSFFLY SKLTVDKSRW QQGNVFSCSV MHEALHNHYT QKSLSLSPGK | |
| | IL-21 | |
| AVC-001 | DLVPEFLPAP EDVETNCEWS AFSCFQKAQL KSANTGNNER IINVSIKKLK RKPPSTNAGR RQKHRLTCPS CDSYEKKPPK | 560 |
| AVC-001 | EFLERFKSLL QKMIHQHLSS RTHGSEDS | 588 |

Heavy chain 1 of AVC-002 (SEQ ID NO: 13) compared with VH3/3-11
(SEQ ID NO: 22) and a human IgG1 constant region (SEQ ID NO: 23)

| | | |
|---|---|---|
| | CDR-H1 CDR-H2 | |
| AVC-002 | EVQLVESGGG LVQPGGSLRL SCAASGFNIK DTYIHWVRQA PGKGLEWVAR IYPTNGYTRY ADSVKGRFTI SADTSKNTAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-002 | LQMNSLRAED TAVYYCSRWG GDGFYAMDYW GQGTLVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-002 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-002 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-002 | EYKCKVSNKA LPAPIEKTIS KAKGQPREPQ VYTLPPCREE MTKNQVSLWC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-002 | LDSDGSFFLY SKLTVDKSRW QQGNVFSCSV MHEALHNHYT QKSLSLSPGK GGGGSGGGGS GGGGSREGPE LSPDDPAGLL | 480 |
| HuIgG1 | LDSDGSFFLY SKLTVDKSRW QQGNVFSCSV MHEALHNHYT QKSLSLSPGK | |
| AVC-002 | DLRQGMFAQL VAQNVLLIDG PLSWYSDPGL AGVSLTGGLS YKEDTKELVV AKAGVYYVFF QLELRRVVAG EGSGSVSLAL | 560 |
| | 4-1BBL ECD | |
| AVC-002 | HLQPLRSAAG AAALALTVDL PPASSEARNS AFGFQGRLLH LSAGQRLGVH LHTEARARHA WQLTQGATVL GLFRVTPEIP | 640 |
| AVC-002 | AGLPSPRSE | 649 |

Heavy chain 1 of AVC-003 and AVC-005 (SEQ ID NO: 14) compared with
VH3/3-11 (SEQ ID NO: 22) and a human IgG1 constant region (SEQ ID NO: 23)

| | | |
|---|---|---|
| | CDR-H1 CDR-H2 | |
| AVC-003 | EVQLVESGGG LVQPGGSLRL SCAASGFNIK DTYIHWVRQA PGKGLEWVAR IYPTNGYTRY ADSVKGRFTI SADTSKNTAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-003 | LQMNSLRAED TAVYYCSRWG GDGFYAMDYW GQGTLVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-003 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-003 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-003 | EYKCKVSNKA LPAPIEKTIS KAKGQPREPQ VYTLPPCREE MTKNQVSLWC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| AVC-003 | LDSDGSFFLY SKLTVDKSRW QQGNVFSCSV MHEALHNHYT QKSLSLSPGK | 450 |
| HuIgG1 | LDSDGSFFLY SKLTVDKSRW QQGNVFSCSV MHEALHNHYT QKSLSLSPGK | |

Heavy chain 2 of AVC-004 and AVC-005 (SEQ ID NO: 15) compared with
VH3/3-11 (SEQ ID NO: 22) and a human IgG1 constant region (SEQ ID NO: 23)

| | | |
|---|---|---|
| | CDR-H1 CDR-H2 | |
| AVC-004 | EVQLVESGGG LVQPGGSLRL SCAASGFNIK DTYIHWVRQA PGKGLEWVAR IYPTNGYTRY ADSVKGRFTI SADTSKNTAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-004 | LQMNSLRAED TAVYYCSRWG GDGFYAMDYW GQGTLVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-004 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-004 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-004 | EYKCKVSNKA LPAPIEKTIS KAKGQPREPQ VCTLPPSREE MTKNQVSLSC AVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Knobs-Into-Hole | |
| AVC-004 | LDSDGSFFLV SKLTVDKSRW QQGNVFSCSV MHEALHNHYT QKSLSLSPGK | 450 |
| HuIgG1 | LDSDGSFFLY SKLTVDKSRW QQGNVFSCSV MHEALHNHYT QKSLSLSPGK | |

Heavy chain of AVC-007 (SEQ ID NO: 18) compared with VH3/3-11
(SEQ ID NO: 22) and a human IgG1 constant region (SEQ ID NO: 23)

| | | |
|---|---|---|
| | CDR-H1 CDR-H2 | |
| AVC-007 | EVQLVESGGG LVQPGGSLRL SCAASGFNIK DTYIHWVRQA PGKGLEWVAR IYPTNGYTRY ADSVKGRFTI SADTSKNTAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-007 | LQMNSLRAED TAVYYCSRWG GDGFYAMDYW GQGTLVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-007 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-007 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ | |
| AVC-007 | EYKCKVSNKA LPAPIEKTIS KAKGQPREPQ VYTLPPSREE MTKNQVSLTC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-007 | LDSDGSFFLY SKLTVDKSRW QQGNVFSCSV MHEALHNHYT QKSLSLSPGK GGGGSGGGGS GGGGSREGPE LSPDDPAGLL | 480 |
| HuIgG1 | LDSDGSFFLY SKLTVDKSRW QQGNVFSCSV MHEALHNHYT QKSLSLSPGK | |
| AVC-007 | DLRQGMFAQL VAQNVLLIDG PLSWYSDPGL AGVSLTGGLS YKEDTKELVV AKAGVYYVFF QLELRRVVAG EGSGSVSLAL | 560 |
| | 4-1BBL ECD | |
| AVC-007 | HLQPLRSAAG AAALALTVDL PPASSEARNS AFGFQGRLLH LSAGQRLGVH LHTEARARHA WQLTQGATVL GLFRVTPEIP | 640 |
| | Linker | |
| AVC-007 | AGLPSPRSEG GGGSGGGGSG GGGSGGGGSR EGPELSPDDP AGLLDLRQGM FAQLVAQNVL LIDGPLSWYS DPGLAGVSLT | 720 |
| | 4-1BBL ECD | |
| AVC-007 | GGLSYKEDTK ELVVAKAGVY YVFFQLELRR VVAGEGSGSV SLALHLQPLR SAAGAAALAL TVDLPPASSE ARNSAFGFQG | 800 |
| | Linker | |
| AVC-007 | GLLHLSAGQR LGVHLHTEAR ARHAWQLTQG ATVLGLFRVT PEIPAGLPSP RSEGGGGSGG GGSGGGGSGG GGSREGPELS | 880 |
| | 4-1BBL ECD | |
| AVC-007 | PDDPAGLLDL RQGMFAQLVA QNVLLIDGPL SWYSDPGLAG VSLTGGLSYK EDTKELVVAK AGVYYVFFQL ELRRVVAGEG | 960 |
| AVC-007 | SGSVSLALHL QPLRSAAGAA ALALTVDLPP ASSEARNSAF GFQGRLLHLS AGQRLGVHLH TEARARHAWQ LTQGATVLGL | 1040 |
| AVC-007 | FRVTPEIPAG LPSPRSE | 1057 |

Heavy chain of AVC-008 (SEQ ID NO: 19) compared with VH3/3-11
(SEQ ID NO: 22) and a human IgG1 constant region (SEQ ID NO: 23)

| | | |
|---|---|---|
| | CDR-H1 CDR-H2 | |
| AVC-008 | EVQLVESGGG LVQPGGSLRL SCAASGFNIK DTYIHWVRQA PGKGLEWVAR IYPTNGYTRY ADSVKGRFTI SADTSKNTAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-008 | LQMNSLRAED TAVYYCSRWG GDGFYAMDYW GQGTLVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-008 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-008 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ | |
| AVC-008 | EYKCKVSNKA LPAPIEKTIS KAKGQPREPQ VYTLPPSREE MTKNQVSLTC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-008 | LDSDGSFFLY SKLTVDKSRW QQGNVFSCSV MHEALHNHYT QKSLSLSPGK GGGGSQGQDR HMIRMRQLID IVDQLKNYVN | 480 |
| HuIgG1 | LDSDGSFFLY SKLTVDKSRW QQGNVFSCSV MHEALHNHYT QKSLSLSPGK | |
| | IL-21 | |
| AVC-008 | DLVPEFLPAP EDVETNCEWS AFSCFQKAQL KSANTGNNER IINVSIKKLK RKPPSTNAGR RQKHRLTCPS CDSYEKKPPK | 560 |
| AVC-008 | EFLERFKSLL QKMIHQHLSS RTHGSEDS | 588 |

Trastuzumab light chain of AVC-001-AVC-005 and AVC-007 and AVC-008
(SEQ ID NO: 2) compared with VK3/A27 (SEQ ID NO: 49)

| | | |
|---|---|---|
| | CDR-L1 CDR-L2 | |
| AVC-001 | DIQMTQSPSS LSASVGDRVT ITCRASQDVN -TAVAWYQQKP GKAPKLLIYS ASFLYSGVPS RFSGSRSGTD FTLTISSLQP | 80 |
| VK3/A27 | EIVLTQSPGT LSLSPGERAT LSCRASQSVS SSYLAWYQQKP GQAPRLLIYG ASSRATGIPD RFSGSGSGTD FTLTISRLEP | |
| | CDR-L3 CL→ | |
| AVC-001 | EDFATYYCQQ HYTTPPTFGQ GTKVEIKRTV AAPSVFIFPP SDEQLKSGTA SVVCLLNNFY PREAKVQWKV DNALQSGNSQ | 160 |
| VK3/A27 | EDFAVYYCQQ YGSSP | |
| AVC-001 | ESVTEQDSKD STYSLSSTLT LSKADYEKHK VYACEVTHQG LSSPVTKSFN RGEC | 214 |

Light chain of AVC-007 (SEQ ID NO: 21) compared with VK3/A27 (SEQ ID NO: 49)

| | | |
|---|---|---|
| | CDR-L1 CDR-L2 | |
| AVC-007 | DIQMTQSPSS LSASVGDRVT ITCRASQDVN -TAVAWYQQKP GKAPKLLIYS ASFLYSGVPS RFSGSRSGTD FTLTISSLQP | 80 |
| VK3/A27 | EIVLTQSPGT LSLSPGERAT LSCRASQSVS SSYLAWYQQKP GQAPRLLIYG ASSRATGIPD RFSGSGSGTD FTLTISRLEP | |
| | CDR-L3 CL→ | |
| AVC-007 | EDFATYYCQQ HYTTPPTFGQ GTKVEIKRTV AAPSVFIFPP SDEQLKSGTA SVVCLLNNFY PREAKVQWKV DNALQSGNSQ | 160 |
| VK3/A27 | EDFAVYYCQQ YGSSP | |
| | Linker | |
| AVC-007 | ESVTEQDSKD STYSLSSTLT LSKADYEKHK VYACEVTHQG LSSPVTKSFN RGECGGGGSQ GQDRHMIRMR QLIDIVDQLK | 240 |
| | IL-21 | |
| AVC-007 | NYVNDLVPEF LPAPEDVETN CEWSAFSCFQ KAQLKSANTG NNERIINVSI KKLKRKPPST NAGRRQKHRL TCPSCDSYEK | 320 |
| AVC-007 | KPPKEFLERF KSLLQKMIHQ HLSSRTHGSE DS | 352 |

CDR and hinge sequences are indicated and underlined.
Constant regions are indicated with arrows.
Linker sequences are indicated and underlined and in italics.
Knobs-into-hole modifications are indicated and underlined, bold and in italics.
Receptor agonist are indicated.

### Example 2 Biochemical Characterization of Multispecific Antigen Binding Proteins

### 2.1. Expression Level and Molecular Weight

Expression levels and molecular weight of the multispecific antigen binding proteins is determined by SDS-PAGE and Size Exclusion Chromatography (SEC). Coomassie blue-stained 4% to 15% SDS-PAGE is performed under both reducing and non-reducing conditions (Zhang et al. Clin Cancer Res, 2007; 13(9): 2758-2767).

Size exclusion chromatography is performed using a Yarra SEC-3000 column (Phenomenex, 00H-4513-K0) and a Waters 2695 HPLC (Waters Corporation) in a 0.1 M Na₂HPO₄/NaH₂PO₄, 0.1 M Na₂SO₄, pH 6.7 mobile phase at a flow rate of 0.5 mL/min. Thyroglobulin (669 kDa, Sr 8.5 nm), b-amylase (200 kDa, Sr 5.4 nm), bovine serum albumin (67 kDa, Sr 3.55 nm), carbonic anhydrase (29 kDa, Sr 2.35 nm) and FLAG peptide (1 kDa) served as standard proteins.

### 2.2 Stability

Stability of the multispecific antigen binding proteins is determined by repeating the above SDS-PAGE and SEC on samples of the multispecific antigen binding proteins that are stored at room temperature for for 1, 3, and 7 days.

Stability of the multispecific antigen binding proteins in plasma is determined by incubating the proteins at 200 nM in 50% human plasma at 37° C. for 1, 3, and 7 days.

Samples are frozen at −20° C. immediately after preparation (0 d) or after the respective incubation period. The level of intact protein is determined using SDS-PAGE and SEC as described above.

### 2.3 Affinity for IL21R

Affinity of the multispecific antigen binding proteins for IL21R “T was analyzed using a Biacore T200 instrument at 25° C. and a flow rate of 50 μl/min. Anti-human Fc antibody was covalently immobilized onto a CM5 sensor chip to capture the multispecific antigen binding proteins in flow cells fc2 and fc4, with fc1 and fc3 as references. IL-21R was introduced at concentrations of 0.74 nM, 2.22 nM, 6.67 nM, 20 nM, and 60 nM, prepared through a series of 1:3 serial dilutions. The assay included a 180-second association phase and a 1200-second dissociation phase after the highest concentration exposure. The analysis buffer contained 10 mM HEPES (pH 7.4), 150 mM NaCl, 0.05% Tween 20, and 3 mM EDTA. Regeneration was performed under standard conditions to remove the complex from all surfaces.

### 2.4 Affinity for 4-1BB

The interaction between the multispecific antigen binding proteins and the 4-1 BB receptor was analyzed utilizing a Biacore T200 instrument at 25° C. with a 50 μl/min flow rate. The CM5 sensor chip from the Human Antibody Capture Kit (Cytiva) was used, and the analysis buffer was composed of 10 mM HEPES (pH 7.4), 150 mM NaCl, 0.05% Tween 20, and 3 mM EDTA. Anti-human Fc antibody was covalently immobilized onto the CM5 sensor chip to capture the multispecific antigen binding proteins on flow cells fc2 and fc4, while fc1 and fc3 served as references. Human 4-1BB was introduced in concentrations of 5 nM, 15 nM, 45 nM, 135 nM, and 405 nM, following a series of 1:3 serial dilutions. The assay protocol included a 120-second association phase and a 600-second dissociation phase post the introduction of the highest concentration sample. Following the interaction analyses, regeneration was conducted under standard conditions to effectively remove the complex from all surfaces.

Recombinant biotinylated human, cynomolgus and murine 4-1BB Fc (kih) fusion molecules (see Example 3 of WO 2016/075278) are directly coupled on a SA chip using the standard coupling instruction (Biacore, Freiburg/Germany). The immobilization level is about 30 RU. The multispecific antigen binding or controls, are passed at a concentration range from 0.39 to 200 nM with a flow of 30 μL/minute through the flow cells over 120 seconds. The dissociation is monitored for 180 seconds. Bulk refractive index differences are corrected for by subtracting the response obtained on a reference empty flow cell.

For affinity measurement, direct coupling of around 7200 resonance units (RU) of an anti-human Fc specific antibody is performed on a CM5 chip at pH 5.0 using the standard amine coupling kit (GF Healthcare). 4-1 BBL-containing multispecific antigen binding proteins or controls, at 50 nM are captured with a flow rate of 30 μL/minute for 60 seconds on flow cell 2. A dilution series (1.95 to 1000 nM) of human 4-1 BB-avi-His (see Example 3 of WO 2016/075278) is passed on both flow cells at 30 μL/minute for 180 sec to record the association phase. The dissociation phase is monitored for 180 sec and triggered by switching from the sample solution to HBS-EP. The chip surface is regenerated after every cycle using a double injection of 60 sec 10 mM Glycine-HCl pH 2.1. Bulk refractive index differences are corrected for by subtracting the response obtained on reference flow cell 1. For the interaction between the 4-1BBL-containing multispecific antigen binding proteins and hu4-IBB avi His, the affinity constants are derived from the rate constants by fitting to a 1:1 Langmuir binding curve using the Biaeval software (GF Healthcare).

### 2.5 Affinity for NKp46

Affinity of the multispecific antigen binding proteins for NKp46 was determined by SPR, essentially as described in Gauthier et al., 2019, Cell 177, 1701-1713.

### Example 3 Functional In Vitro Characterization of Multispecific Antigen Binding Proteins

### 3.1 Binding to NK Cells

Binding of the multispecific antigen binding proteins to NK cells is demonstrated using flow cytometry and competitive inhibition with unlabeled competing antibodies, essentially as described by Fellermeier et al. (2016, supra). Assays are performed in triplicate against NK cells from four different donors. Mean fluorescent intensity is plotted against dilutions of multispecific antigen binding proteins to determine EC50 and EC90 concentrations for optimal effective engaging.

### 3.2 Short Term NK Cell Cytotoxicity

Short term (4-hour) NK cell cytotoxicity of the multispecific antigen binding proteins was determined using the calcein-acetoxymethyl (calcein-AM) release assay (Somanchi et al., 2011, J Vis Exp., 2:2540; Lee et al., 2010, Methods Mol. Biol., 651:61-77). Target cells (SKOV-3, MDA-MB-231 or K562 cells) were loaded with 0.025 μM Calcein-AM and incubated for 1 hour at 37° C. with gentle resuspension every 10-15 minutes. After this target cells were washed twice with medium and 10,000 target cells were seeded in a 96-well plate. NK cells were purified via negative selection using RosetteSep (In: Methods in Molecular Biology, Ex Vivo Expansion of Human NK Cells Using K562 Engineered to Express Membrane Bound IL21 Srinivas S. Somanchi and Dean A. Lee DOI 10.1007/978-1-4939-3684-7) from four normal donor buffy coats and expanded ex vivo at effector-to-target (E:T) ratio of 0.5:1, with and without (saturating concentration) of the multispecific antigen binding proteins AVC-001 or AVC-007 as described in Example 3.5 below. Assays are performed in triplicate. Cytotoxicity of fresh NK cells (above) is compared with cytotoxicity of NK cells expanded ex vivo for 1, 2 or 3 weeks in the presence of a multispecific antigen binding protein (AVC-001 or AVC-007). NK cells were taken from the expansions and added in an E:T ration of 2:1 to the target cells, and co-cultured for 4 hours at 37° C. In addition two control conditions are setup as well:
1. Target cells only—this sample was used to quantify the spontaneous release of Calcein-AM from the tumor cells
2. Maximum release—this sample was treated with Triton X-100 to kill and permeabilize the target cells and used to quantify the maximum possible release of Calcein-AM
   After this incubation supernatant was transferred to a new plate and fluorescence was measured on a plate reader using an excitation filter at 485 nm and an emission filter at 530 nm. Specific lysis was calculated using the following formula:

$$\% Specific Lysis = \frac{( {Fluorescence}_{Experimental} - {Fluorescence}_{Spontaneous} )}{( {Fluorescence}_{Maximum} - {Fluorescence}_{Spontaneous} )} \times 100$$

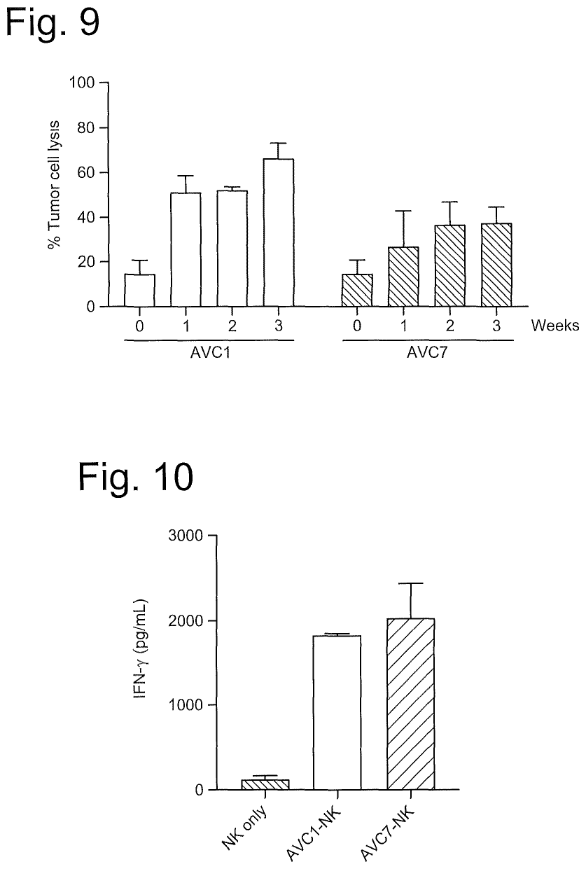

The results are shown in FIG. 9 and demonstrate a significant increase in cytotoxicity against MDA-MB-231 target cells of NK cell cells expanded in the presence of the multispecific antigen binding proteins AVC-001 or AVC-007, as compared to the cytotoxicity of fresh NK cells. Similar data were obtained with SKOV-3 and K562 cells as target cells (data not shown).

### 3.3.1 Long-Term NK Cell Cytotoxicity

Long-term NK cell cytotoxicity of the multispecific antigen binding proteins, to assess serial killing, was determined using the xCelligence assay (see e.g. Naeimi Kararoudi et al., 2022, Cell Reports Methods 2, 100236 Jun. 20, 2022). Target cells are SKOV-3 (High HER2) and MDA-MB-175 VII (low HER2). NK cells were purified via negative selection using RosetteSep (see above) from four normal donor buffy coats.

First, 50 μL of target cell culturing medium was added to each well of 96 well E-Plates (ACEA Biosciences) and the background impedance was measured and displayed as Cell Index. Dissociated adherent SKOV-3 or MDA-MB-231 cells were seeded at a density of 10,000 cells/well of the E-Plate in a volume of 100 μL and allowed to passively adhere on the electrode surface. Post seeding, the E-Plate was kept at ambient temperature inside a laminar flow hood for 30 minutes and then transferred to the RTCA MP instrument inside a cell culture incubator. Data recording was initiated immediately at 15 minute intervals for the entire duration of the experiment. After 4 hours, data acquisition was paused, 100 μL of medium was removed from each well and 20,000 purified NK cells were added in a volume of 100 μL (E:T ratio of 2:1). Immediately prior to addition of effector cells, the multispecific antigen binding proteins AVC-001 or AVC-007, or the control proteins AVC-003 (trastuzumab analog with only IL-21) or AVC-005 (trastuzumab analog without cytokines), were added at a concentration of 25 nM, which is expected to be a saturating concentration.

Changes in impedance were reported as Cell Index (CI) which was normalized to the values on t=0 and then used to calculate the % Tumor cell lysis, using the formula below:

$$\% \cdot Cytolysis = [ 1 - \frac{{CI}_{ti} / {CI}_{nml \_ time}}{( \overline{{CI}_{TargetAlone ti} / {CI}_{TargetAlone nml \_ time}} )} ] \times 100$$

wherein: CIᵤ is the average Cell Index between replicate wells at the time ti; CI_{nml_time} is the average Cell Index between replicate wells at normalization time; CI_{TargetAlont ti} is the average Cell Index between replicate target alone control wells at the time ti; CI_{Targetalone nml_time} is the average Cell Index between replicate target alone control wells at normalization time.

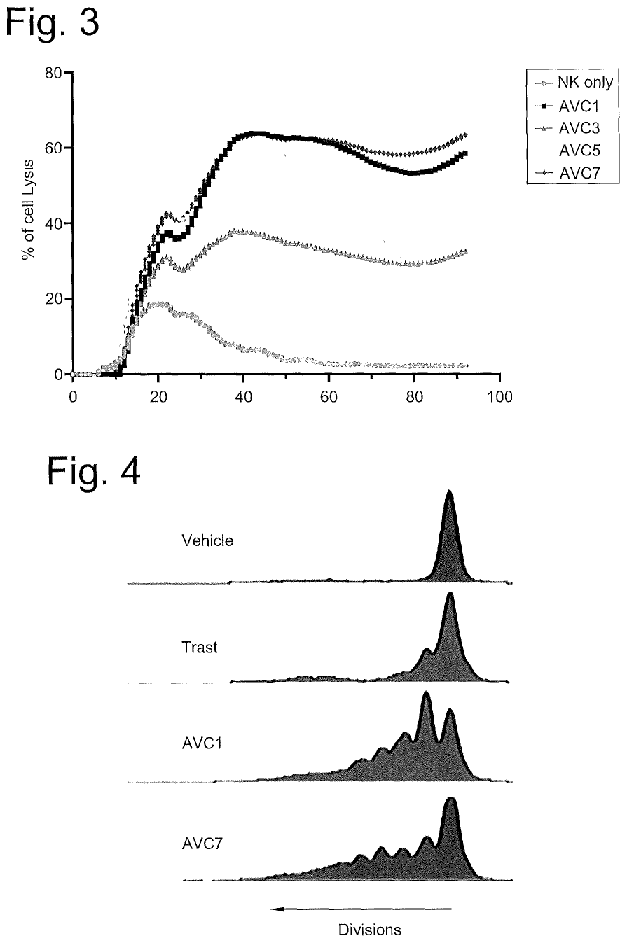

The results obtained with SKOV-3 as target cells are shown in FIG. 3. The multispecific antigen binding proteins AVC-001 and AVC-007, which contain both the 4-1BBL and IL-21 cytokines, significantly outperform the controls both in strength and duration of the cytotoxic effect against the SKOV-3 cells. Controls were NK cells only or control proteins AVC-003 or AVC-005. Similar results were obtained with MDA-MB-231 as target cells.

### 3.3.2 Long-Term Repeated NK Cell Cytotoxicity

Anti-tumor activity of NK cells, and specifically ADCC, is typically a short-term process taking place within 4 hours of start of co-culture. However, the multispecific antigen binding protein disclosed herein are expected to induce a longer term cytoxic effect, e.g. after at least 40-60 hours of co-culture. Therefore a repeated co-culture system was set up, similar to the set-up used by Thakur et al. (J Cancer Res Clin Oncol. 2020 August; 146(8): 2007-2016.) to study repeated cytotoxicity of CAR-Ts with a HER2-EGFR bi-specific binder.

Since for these longer term measurements an idea of general cell culture state is desirable, an Incucyte® Live-Cell Analysis System was used. SKOV-3 target cells and NK cells (purified via negative selection using RosetteSep from normal donor buffy coats as above) were used in an E:T ratio of 1:1. The SKOV-3 target cells were lentivirally transduced with Nuclight Red (Sartorius Cat #4625) to allow read-out of fluorescently labeled target cell counts. Assays were performed in triplicate.

A fixed amount of 10,000 fluorescently labeled target cells in 200 μl medium per well was used to ensure a sufficient amount of nutrients for at least 3 days of culture. Co-culture was performed in the presence of 50 IU/mL IL-2. After 3 days, NK cells were harvested and used to setup a new round of co-culture with fresh target cells in a 1:1 E:T ratio. 25 nM of the multispecific antigen binding proteins AVC-001 or AVC-007 or the AVC-005 control protein were added at the start of each 3 day round to expose NK cells to new booster proteins at the beginning of every round. Control wells contained only NK cells and SKOV-3 target cells, or only SKOV-3 target cells. Cells were monitored and fluorescently labeled target cells were counted every 3 hours. The experiment was continued for 6 co-culture rounds, i.e., for 18 days.

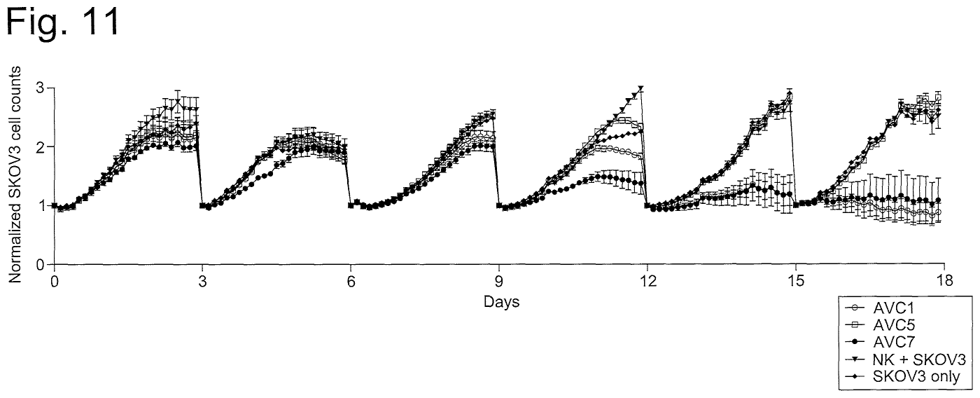

The results are presented in FIG. 11. Control wells with only NK cells and SKOV-3 target cells, or only SKOV-3 target cells show unhindered growth of tumor cells. Already after the first 3 day cycle the AVC-005 Trastuzumab analog control protein starts to loose effective controls of tumor cell growth. By contrast, the multispecific antigen binding proteins AVC1 and AVC7 significantly control tumor cell numbers in later cycles, especially after 9 days and on.

### 3.4 NK Cell Proliferation Assay

NK cells were labeled with CellTrace Violet Cell Proliferation Dye and expanded in presence of target tumor cell line SKOV-3 at an E:T ratio of 1:1, without and with the multispecific antigen binding proteins AVC-001 or AVC-007, or the AVC-005 Trastuzumab analog as control protein (each protein at four dose levels: 0,025, 0.25, 2.5 or 25 nM). Assays were performed in triplicate. After 96 hours cells are harvested, stained for viability with Live/Dead stain and for surface markers to gate on the viable CD3-CD56′ NK cell population. The dilution of CellTrace dye in FIG. 4 shows that AVC-001 and AVC-007 induce increased and extended proliferation over the AVC-005 Trastuzumab analog. Each peak in FIG. 4 represents a division. Vehicle control is a co-culture of NK cells with target cells without the addition of multispecific antigen binding proteins or control protein.

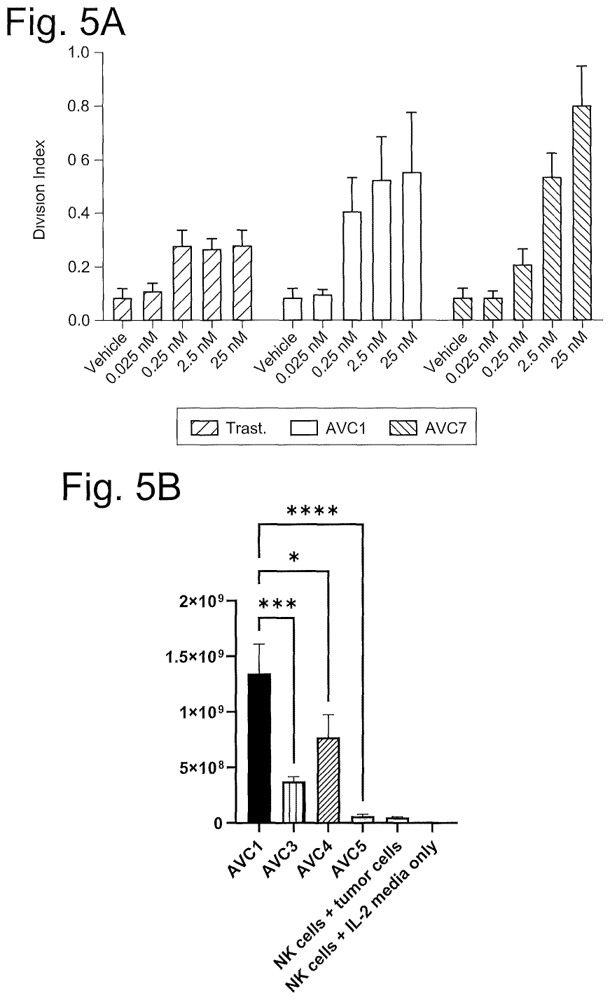

FIG. 5A represents a graph of the dose-responses for AVC-001 and AVC-007-induced proliferation of NK cells at indicated concentrations, compared to that of the AVC-005 Trastuzumab analog control protein. The division index is the average number of divisions that a cell in the original population has undergone, including the cells which never divided. It is clear that AVC-001 and AVC-007 induce stronger proliferation of NK cells as compared to the AVC-005 control protein.

### 3.5 Ex Vivo Expansion of NK Cells

Protocols used for ex vivo expansion of donor NK cells are essentially as described in Denman et al. (2012, supra).

Briefly, NK cells were isolated from 4 healthy donor buffy coats with RosetteSep enrichment and Ficoll (GE HealthCare, Piscataway, NJ). At day 0, NK cells were stimulated as follows: per 0.5×10⁶ NK Cells, 1×10⁶ irradiated SKOV3 cells were pre-incubated with each of the multispecific antigen binding proteins AVC-001 or AVC-007, or the AVC-005 Trastuzumab analog as control protein for 30 minutes at 4° C. SKOV3 cells were incubated in 1 μg of AVC-protein per mL of medium, whereby SKOV3 cells were resuspended at 1×10⁶ per mL of medium. SKOV3 cells were then washed and added directly to the NK Cells at an E:T ratio of 0.5:1 (NK cell to SKOV3) per donor. SKOV3 cells were incubated in 1 μg of AVC-protein per mL of medium, whereby SKOV3 cells were resuspended at 1×10⁶ per mL of medium. As control NK cells were stimulated with irradiated (100 cGy) FC21 feeder cells at a ratio of 1:2 (NK:FC21).Cells were plated at a density were then plated at a density of 0.2×10⁶ cells/mL in 5 mL of AIM V™ media (12055091, Gibco, Thermo Scientific) supplemented with CTS™ immune cell serum replacement (A2596101, Gibco, Thermo Scientific) and with 50 IU/mL recombinant human IL-2 (Proleukin, Novartis Vaccines and Diagnostics, Inc).

At the end of each week, 0.5×10⁶ NK Cells were stimulated in the exact same fashion as they were on day 0 except at a 1:1 ratio. (1:1 NK cell to FC21 or 1:1 NK cell to SKOV3). Cells are plated in 5 mL of AIM V media supplemented with CTS immune cell serum replacement with 50 IU/mL human IL-2. If the cells did not expand/proliferate or they died off, that specific condition/donor was discontinued.

Every other day of expansion, NK Cells are counted using a Nexcelom Cellometer and AOPI staining solution. Total cell counts are determined. AIM V media supplemented with CTS immune cell serum replacement is added to keep the cells around a concentration of 0.3×10⁶ NK cells/mL of medium. 50 IU/mL human IL-2 is added to each condition/donor.

FIG. 5B represents a graph comparing the different rates of NK cell proliferation induced by co-culture with SKOV-3 tumor cells in the absence or presence of AVC-001, AVC-003, AVC-004 or AVC-005, all at 1.6 nM. All molecules induce proliferation compared to have control condition where NK cells were co-cultured with SKOV3 without test articles present. However, proliferation induced by the multispecific antigen binding protein comprising both NK cell-activating cytokines IL-21 and 4-1BBL (AVC-001) is superior to that induced by proteins comprising either AVC3 (IL-21), AVC4 (4-1BBL) or none (AVC-005) of the cytokines.

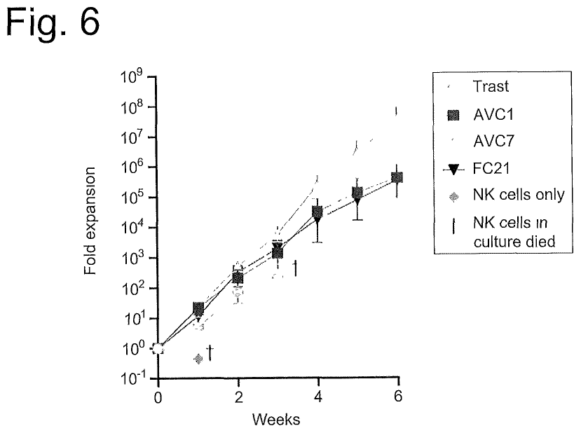

FIG. 6 shows the fold expansion of the NK cells over the course of up to 6 weeks under the various conditions of stimulation. Unstimulated NK cells died after one week. NK cells stimulated with the AVC-005 Trastuzumab analog lacking cytokines died after three weeks. Stimulation and activation of NK cells by the soluble AVC-001 or AVC-007 multispecific antigen binding proteins was at least comparable (AVC-001) or higher (AVC-007) than the reference FC21 feeder cells that express membrane-bound IL-21 and 4-1BBL.

### Example 4 Phenotypic Characterization of NK Cells

### 4.1 Cytokine Secretion by Expanded NK Cells

NK cells were co-cultured for 7 days with SKOV-3 cells at a 1:1 ratio in the absence and presence of saturating concentrations of the multispecific antigen binding proteins AVC-001 or AVC-007. Supernatants are collected and the concentration of IFN-γ was determined using the AlphaLISA method (Perkin Elmer). Concentrations were calculated from fluorescent intensity based on standard curves and formulas provided with the kit. FIG. 10 shows that NK cells co-cultured for 7 days with SKOV-3 cells in the presence of AVC-001 or AVC-007 resulted in a marked increase in IFN-γ production as compared to NK cells co-cultured in their absence.

### 4.2 Transcriptome Analysis of Expanded NK Cells

Gene expression in NK cells stimulated with the multispecific antigen binding proteins is assessed using the nCounter platform (nanoString Technologies, Inc., Seattle, WA; Geiss et al. 2008, Nat Biotechnol 26: 317-325). Purified NK cells from four donors are stimulated for one week in parallel expansions with either a multispecific antigen binding protein or FC21 feeder cells (Denman et al. pLoS ONE, 2012, supra). Total RNA is purified from each sample and assessed for expression of 96 genes (Denman et al. pLoS ONE, 2012, supra). Gene expression is normalized to LDH (mean 6,076 copies detected from 100 ng of loaded mRNA), and plotted as mean±SEM. Genes with borderline detection (≤10 normalized transcripts detected) are excluded from further analyses. Genes having 2-fold difference in mean expression between cultures with the multispecific antigen binding protein and FC21 are then identified.

### 4.3 Analysis of NK-Cell Phenotypes by Multiparameter Mass Cytometry

NK-cell phenotypes were identified by multiparameter mass cytometry. At the start of the expansion and after week 3 of the expansion in the presence of the multispecific antigen binding proteins AVC-001 or AVC-007, or FC21 feeder cells (see Example 3.5), NK cells were collected and incubated with the antibodies listed in Table 4.3.1. Following this, cells were fixed in 2% formaldehyde in PBS and stored in this solution until acquisition.

TABLE 4.3.1
Antibodies conjugated to stable metal isotopes as used for
mass cytometry. All the antibodies were either purchased pre-
conjugated from Fluidigm or were conjugated using X8 MaxPar
conjugation kits according to the manufacture”s protocol.

| Antibody target | Metal tag | Antibody target | Metal tag |
|---|---|---|---|
| CD56 | 163Dy | TIGIT | 157Gd |
| CD3 | 113In | CXCR4 | 158Gd |
| CD45 | 115In | NKp30 | 159Tb |
| CD16 | 207Pb | CXCR6 | 160Gd |
| CD57 | 176Yb | Ki67 | 161Dy |
| NKG2C | 142Ce | NKp46 | 162Dy |
| cPARP | 143Nd | Fas-L | 164Dy |
| CD137 | 145Nd | LAG3 | 165Ho |
| NKp44 | 146Nd | NKG2D | 166Er |
| CD161 | 148Nd | KIR3DL1 | 167Er |
| CD244 | 149Sm | NKG2A | 169Tm |
| CD62L | 150Nd | TIM3 | 170Er |
| CD107a | 151Eu | DNAM-1 | 171Yb |
| KIR2DL1 | 152Sm | NKp80 | 172Yb |
| HLA-DR | 153Eu | KIR2DL2 | 173Yb |
| CD69 | 154Sm | CD94 | 174Yb |
| CD27 | 155Gd | Perforin | 175Lu |
| CXCR3 | 156Gd | | |

Immediately prior to acquisition, the samples were washed with Cell Staining Media (CSM; PBS+0.5% FBS), and resuspended in 1:20 dilution of EQ™ Four Element Calibration Beads (Fluidigm) at a concentration of 1 million cells/mL in CSM as previously described (Rahman et al., 2016, Cytometry A, 89:601-607).

After acquisition on a Helios mass cytometer (Fluidigm), the resulting FCS files were normalized using a normalization tool developed by Finck et al. and analyzed on Cyto-bank (www.cytobank.org) (Finck et al., 2013, Cytometry A, 83(5):483-94; Kotecha et al., Curr Protoc Cytom. 2010 July; Chapter 10: Uniti0.17).

Once the singlet gate was established, cells were identified using markers (listed in Table 4.3.1) and analyzed using viSNE (a visualization tool for high-dimensional single-cell data based on the t-Distributed Stochastic Neighbor Embedding (t-SNE) algorithm) (see Amir et al., 2013, Nat. Biotechnol. 31(6): 545-552).

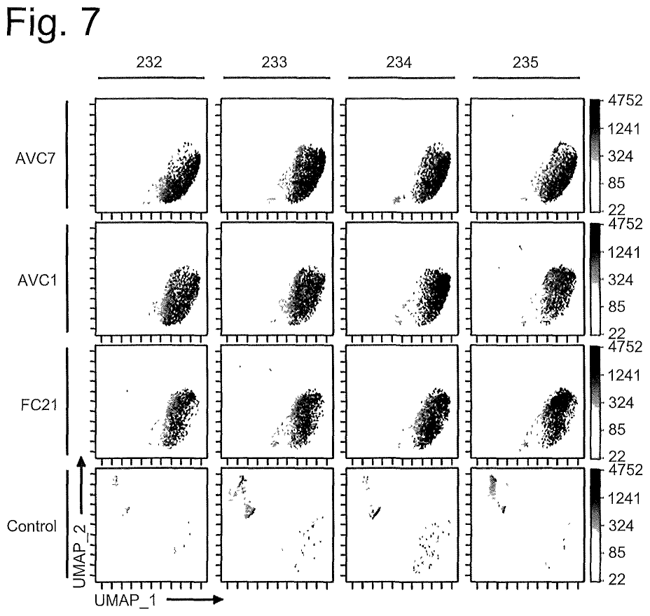

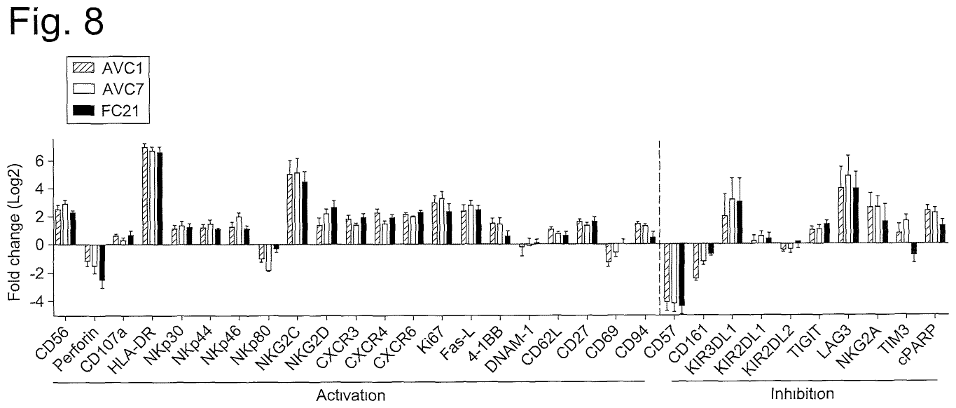

The results in FIGS. 7 and 8 show that the phenotype of NK cells stimulated and activated by AVC-001 or AVC-007 during expansion, closely matches the previously described hyper-functional phenotype of NK cells stimulated and activated by the reference FC21 feeder cells (Denman et al. (2012, supra).

### Example 5 In Vivo Characterization of Multispecific Antigen Binding Proteins

In vivo PK and biodistribution of multispecific antigen binding proteins is performed using whole body clearance studies in BALB/c mice with radiolabeled fusion proteins as described in Zhang et al. (Olin Cancer Res, 2007, supra).

### Example 6 Characterization of Multispecific Antigen Binding Proteins Targeted Against TROP2, GD2, FOLR1, B7-H3, EGFR and PD-L1

### 6.1 Preparation of Further Multispecific Antigen Binding Proteins

Expression constructs comprising coding sequences for further multispecific antigen binding proteins against TAA's other than HER2, as listed in Table 6.1, were generated as described in Example 1.

TABLE 6.1
Multispecific antigen binding proteins against the
TAA's TROP2, GD2, FOLR1, B7-H3, EGFR and PD-L1.

| | Fab | | | SEQ |
|---|---|---|---|---|
| Name | target | Polypeptide chains | | ID NO |
| AVC-016 | TROP2 | Heavy 1 | sacituzumab-Fab + | 168 |
| | | | trastuzumab-Fc + 4-1BBL trimer | |
| | | | fusion | |
| | | Heavy 2 | sacituzumab-Fab + | 169 |
| | | | trastuzumab-Fc + IL-21 fusion | |
| | | Light | sacituzumab Fab | 170 |
| AVC-017 | GD2 | Heavy 1 | dinutuximab-Fab + | 171 |
| | | | trastuzumab-Fc + 4-1BBL trimer | |
| | | | fusion | |
| | | Heavy 2 | dinutuximab-Fab + | 172 |
| | | | trastuzumab-Fc + IL-21 fusion | |
| | | Light | dinutuximab Fab | 173 |
| AVC-018 | FOLR1 | Heavy 1 | mirvetuximab-Fab + | 174 |
| | | | trastuzumab-Fc + 4-1BBL trimer | |
| | | | fusion | |
| | | Heavy 2 | mirvetuximab-Fab + | 175 |
| | | | trastuzumab-Fc + IL-21 fusion | |
| | | Light | mirvetuximab Fab | 176 |
| AVC-019 | B7-H3 | Heavy 1 | omburtamab-Fab + | 177 |
| | | | trastuzumab-Fc + 4-1BBL trimer | |
| | | | fusion | |
| | | Heavy 2 | omburtamab-Fab + | 178 |
| | | | trastuzumab-Fc + IL-21 fusion | |
| | | Light | omburtamab Fab | 179 |
| AVC-020 | EGFR | Heavy 1 | cetuximab-Fab + | 180 |
| | | | trastuzumab-Fc + 4-1BBL trimer | |
| | | | fusion | |
| | | Heavy 2 | cetuximab-Fab + | 181 |
| | | | trastuzumab-Fc + IL-21 fusion | |
| | | Light | cetuximab Fab | 182 |
| AVC-021 | PD-L1 | Heavy 1 | avelumab-Fab + | 183 |
| | | | trastuzumab-Fc + 4-1BBL trimer | |
| | | | fusion | |
| | | Heavy 2 | avelumab-Fab + | 184 |
| | | | trastuzumab-Fc + IL-2 fusion | |
| | | Light | avelumab Fab | 185 |

In each of AVC-016 to AVC-021, the trastuzumab Fc region of heavy chain 1 comprises the knob-into-hole modifications that are also present in heavy chain 1 of AVC-001 (SEQ ID NO: 11) and the trastuzumab Fc region of heavy chain 2 comprises the complementary knob-into-hole modifications that are also present in heavy chain 2 of AVC-001 (SEQ ID NO: 12) (see Table 1.1.2 above).

Expression constructs encoding of AVC-016 to AVC-021 were transfected into HEK293E-253 cells and the multispecific antigen binding proteins were produced, harvested, purified and analyzed as described in Example 1. For comparison multispecific antigen binding proteins AVC-001 and AVC-007, and the trastuzumab analog AVC-005 as control protein produced as described in Example 1 were used.

### 6.2 NK Cell Cytotoxicity

Long-term NK cell cytotoxicity induced by the multispecific antigen binding proteins AVC-001 and AVC-016 to AVC-021 was determined essentially as described in Example 3.3.2. Briefly, NK cell cytotoxicity was determined against tumor cell lines expressing a TAA specifically bound by the multispecific antigen binding proteins AVC-001 and AVC-016 to AVC-021 as indicated in Table 6.2. A single round of co-culture of NK cells and target cells, in a E:T ratio of 2:1 (20,000 NK cells: 10,000 tumor cells) was followed for 96 hours, in the absence or presence of the multispecific antigen binding proteins at 25 nM. Control wells thus contained only NK cells and the respective target tumor cells. Assays were performed in triplicate. Interferon-gamma levels were determined in the supernatant using the MACSplex cytotoxic IFN-γ kit (cat #130-125-800).

TABLE 6.2
Target tumor cell lines used for determination of NK
cell cytotoxicity induced by the multispecific antigen
binding proteins AVC-001 and AVC-016 to AVC-021.

| Multispecific | | | |
|---|---|---|---|
| antigen-binding | | | Target tumor |
| protein | Target antigen | UNIPROT accession | cell line |
| AVC-001 | HER2 | P04626 | SKOV-3 |
| AVC-016 | TROP2 | P09758 | BxPC3 |
| AVC-017 | GD2 | N/A | U87MG |
| AVC-018 | FOLR1 | P15328 | SKOV-3 |
| AVC-019 | B7-H3 | Q5ZPR3 | U87MG |
| AVC-020 | EGFR | P00533 | A431 |
| AVC-021 | PD-L1 | Q9NZQ7 | MDA-MB-231 |

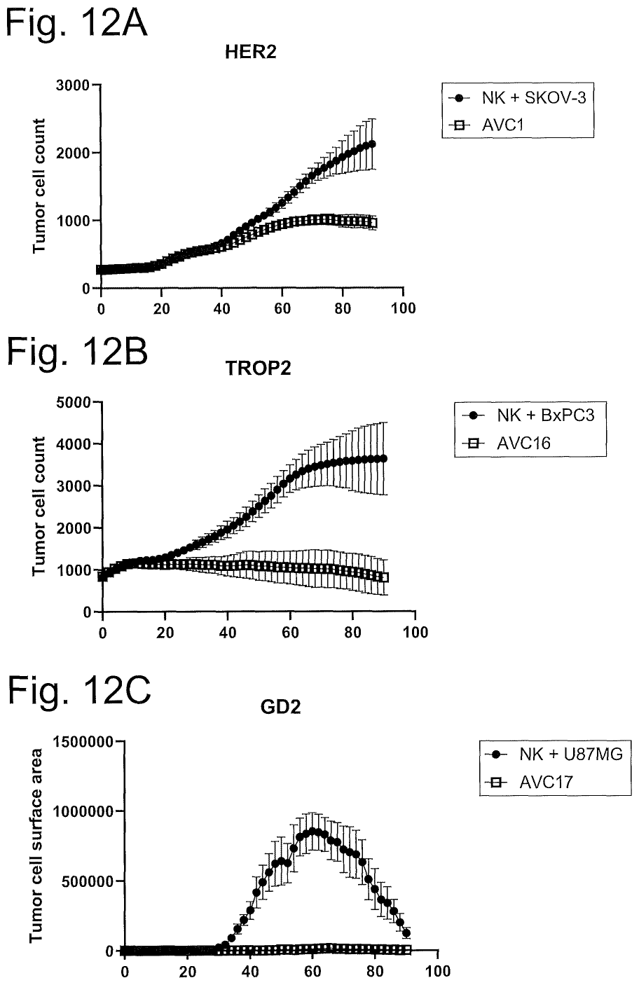

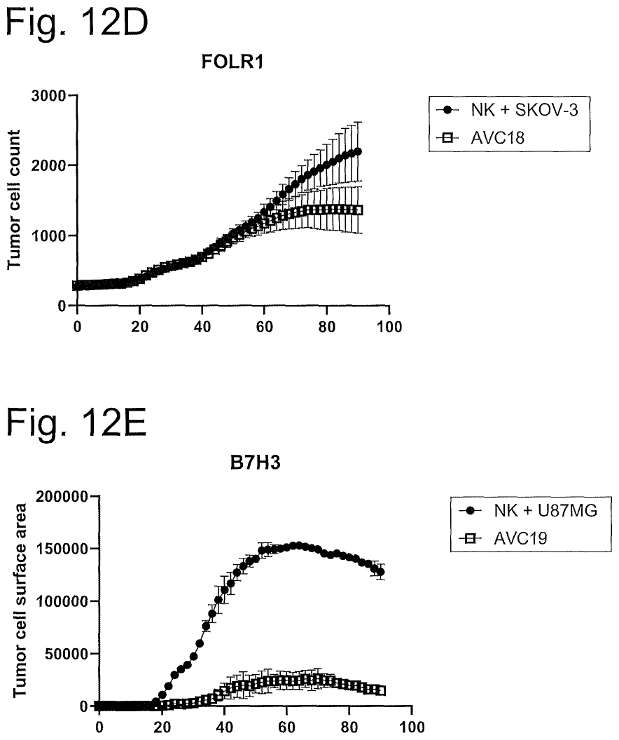

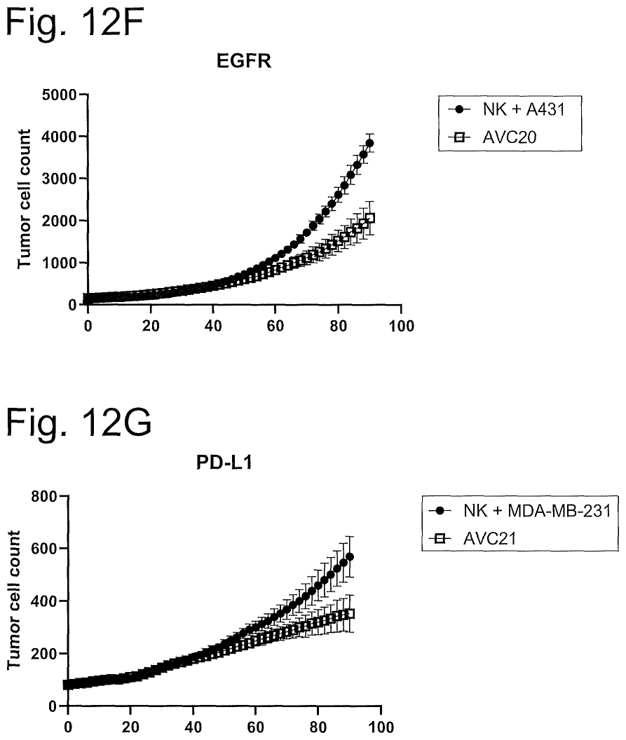

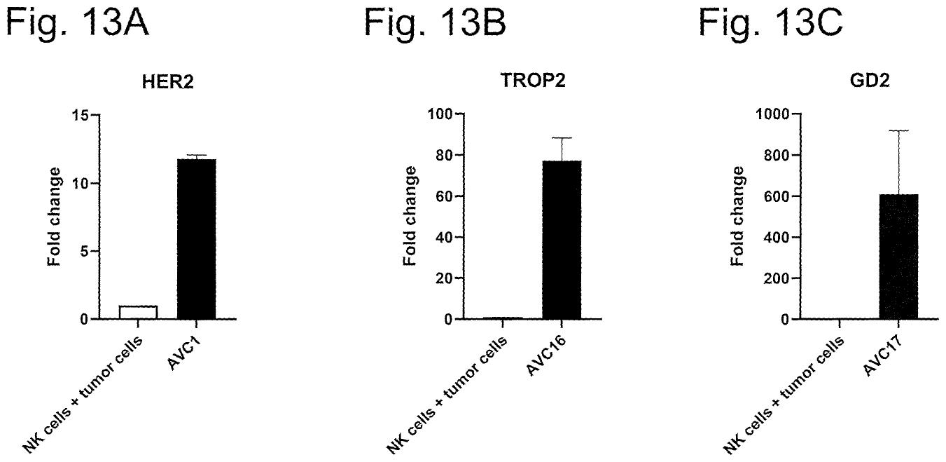

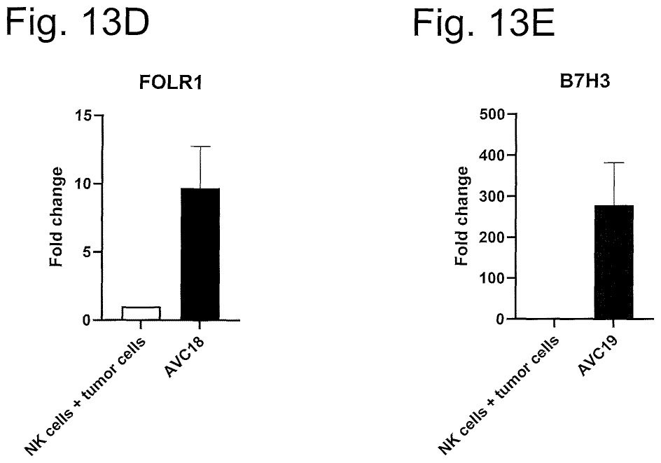

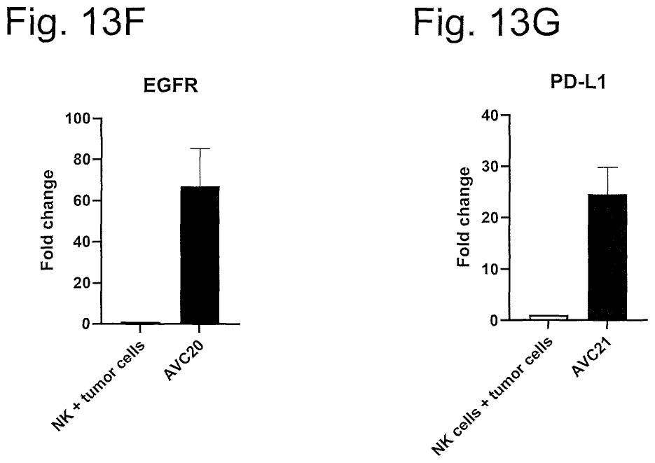

The results for AVC-001 and AVC-016 to AVC-021 are shown in FIGS. 12A to 12G, respectively. Each of the multispecific antigen binding proteins AVC-001 and AVC-016 to AVC-021 significantly induce increased cytotoxicity against tumor cells that express the respective antigens compared to NK cells only. Similarly, FIGS. 13A to 13G show that the same set of multispecific antigen binding proteins induce increase interferon-gamma production in response to co-culture with tumor cells that express the respective antigens. Hence, the stimulatory effects of the multispecific antigen binding proteins are achieved when targeting a variety of different tumor-associated antigens.

### Embodiments

1. A multispecific antigen binding protein comprising:
a) a first antigen-binding region that specifically binds a tumor associated antigen (TAA);
b) a second antigen-binding region that has affinity for a surface antigen expressed on natural killer (NK) cells; and,
c) an NK cell-activating cytokine that is at least one of:
   i) an interleukin 21 receptor (IL21R) agonist; and,
   ii) a 4-1 BB agonist.
      2. A multispecific antigen binding protein according to embodiment 1, wherein the first antigen-binding region comprises at least one immunoglobulin-derived antigen-binding region.
      3. A multispecific antigen binding protein according to embodiment 2, wherein the immunoglobulin-derived antigen-binding region comprises or consists of a Fab or an immunoglobulin single variable domain (ISVD).
      4. A multispecific antigen binding protein according to any one of the preceding embodiments, wherein the first antigen-binding region is a human or humanized antigen-binding region.
      5. A multispecific antigen binding protein according to any one of the preceding embodiments, wherein the multispecific antigen binding protein further comprises a third antigen-binding region that specifically binds a TAA or that specifically binds an NK cell activating receptor.
      6. A multispecific antigen binding protein according to embodiment 5, wherein the first and third antigen-binding regions bind the same TAA or at least two different TAAs.
      7. A multispecific antigen binding protein according to embodiment 6, wherein the first and third antigen-binding regions are identical.
      8. A multispecific antigen binding protein according to any one of the preceding embodiments, wherein the TAA is selected from the group consisting of: Her2 (ErbB2/Neu), Receptor Tyrosine Kinase-like Orphan Receptor 1 (ROR1), Crypto, CD2, CD4, CD20, CD30, CD19, CD38, CD40, CD47, Glycoprotein NMB, CanAg, CD22 (Siglec2), CD33 (Siglec3), CD79, CD123, CD138, CD171, CTLA-4 (CD152), PD1, PSCA, L1-CAM, EpCAM, PSMA (prostate specific membrane antigen), BCMA, TROP2, STEAP1, CD52, CD56, CD80, CD70, E-selectin, EphB2, EPHA4, Melanotransferrin, Mud 6, TMEFF2, Killer Ig-Like Receptor, Killer Ig-Like Receptor 3DL2 (KIR3DL2), B7.1, B7.2, B7-H3, B7-H4, B7-H6, PD-L1, IL-6 receptor, IL-1 accessory Protein, MAGE, MART-1/Melan-A, gp100, MICA, MICB, adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), NaPi2b, TYRP1, nectin-4, a UL16-binding protein (ULBP), a RAET1 protein, carcinoembryonic antigen (CEA), CEACAM5, etv6, aml1, prostate specific antigen (PSA), T-cell receptor/CD3-zeta chain, MAGE-A3, a GAGE-tumor antigen, anti-Müllerian hormone Type II receptor, delta-like ligand 3 (DLL3), delta-like ligand 4 (DLL4), DR5, NTRKR1 (EC 2.7.10.1), SLAMF7, TRAILR1, TRAILR2, BAGE, RAGE, LAGE-1, NAG, GnT-V, MUM-1, CDK4, MUC1, MUC1-C, VEGF, VEGFR2, Angiopoietin-2, PDGF, TGF-alpha, EGF, EGF receptor (EGFR/ERBB1), HER-3/ERBB3, HER-4/ERBB4, a heterodimeric receptor comprised of at least one HER subunit, gastrin releasing peptide receptor antigen, cMET, CA125, integrin receptors, α5β integrins, α5β1 integrins, αIIbβ3-integrins, PDGF alpha receptor, PDGF beta receptor, sVE-cadherin, IL-8 receptor, hCG, IL-6 receptor, IL-1 accessory protein, CSF1R, α-fetoprotein, mesothelin (MSLN), Isoform 2 of Claudin-18 (Claudin 18.2, CLDN18), folate receptor alpha (FRα, FOLR1), tissue factor (TF, CD142), P-cadherin, E-cadherin, α-catenin, β-catenin and γ-catenin, Plexin-A1, TNFRSF10B, AXL, EDNRB, OLR1, ADAM12, PLAUR, CCR4, CCR6, p120ctn, PRAME, NY-ESO-1, cdc27, CDCP1, adenomatous polyposis coli protein (APC), fodrin, Connexin 37, Ig-idiotype, p15, gp75, a GM2 ganglioside, a GD2 ganglioside, a human papillomavirus protein, imp-1, P1A, EBV-encoded nuclear antigen (EBNA)-I, brain glycogen phosphorylase, SSX-1, SSX-2 (HOM-MEL-40), SSX-1, SSX-4, SSX-5, SCP-1 CT-7, c-erbB-2, FcRL5/FcRH5, Flt3, muc16, muc17, mmp9, FAP, Lewis-Y, EGFRvIII, GPC3, GPRC5D, gpA33, 5T4, SSTR2, CD73, CD25, CD45, and CD133.
      9. A multispecific antigen binding protein according to embodiment 8, wherein at least one of the first and third antigen-binding regions comprises a combination of complementarity-determining regions (CDRs) CDR-H1, CDR-H2, CDR-H3, CDR-L1, CDR-L2 and CDR-L3 selected from the group consisting of:
a) the CDR-H1, CDR-H2 and CDR-H3 sequences as comprised in SEQ ID NO: 1, and the CDR-L1, CDR-L2 and CDR-L3 sequences as comprised in SEQ ID NO: 2;
b) the CDR-H1 (SEQ ID NO: 152), CDR-H2 (SEQ ID NO: 153) and CDR-H3 (SEQ ID NO: 154) sequences as comprised in SEQ ID NO: 59, and the CDR-L1 (SEQ ID NO: 155), CDR-L2 (SEQ ID NO: 156) and CDR-L3 (SEQ ID NO: 157) sequences as comprised in SEQ ID NO: 60 (atezolizumab);
c) the CDR-H1 (SEQ ID NO: 158), CDR-H2 (SEQ ID NO: 159) and CDR-H3 (SEQ ID NO: 160) sequences as comprised in SEQ ID NO: 9, and the CDR-L1 (SEQ ID NO: 161), CDR-L2 (SEQ ID NO: 162) and CDR-L3 (SEQ ID NO: 163) sequences as comprised in SEQ ID NO: 10 (avelumab);
d) the CDR-H1 (SEQ ID NO: 164), CDR-H2 (SEQ ID NO: 165) and CDR-H3 (SEQ ID NO: 166) sequences as comprised in SEQ ID NO: 61, and the CDR-L1 (SEQ ID NO: 167), CDR-L2 (SEQ ID NO: 168) and CDR-L3 (SEQ ID NO: 169) sequences as comprised in SEQ ID NO: 62 (durvalumab);
e) the CDR-H1, CDR-H2 and CDR-H3 sequences as comprised in SEQ ID NO: 3, and the CDR-L1, CDR-L2 and CDR-L3 sequences as comprised in SEQ ID NO: 4;
f) the CDR-H1, CDR-H2 and CDR-H3 sequences as comprised in SEQ ID NO: 5, and the CDR-L1, CDR-L2 and CDR-L3 sequences as comprised in SEQ ID NO: 6;
g) the CDR-H1, CDR-H2 and CDR-H3 sequences as comprised in SEQ ID NO: 7, and the CDR-L1, CDR-L2 and CDR-L3 sequences as comprised in SEQ ID NO: 8;
h) the CDR-H1, CDR-H2 and CDR-H3 sequences as comprised in SEQ ID NO: 63, and the CDR-L1, CDR-L2 and CDR-L3 sequences as comprised in SEQ ID NO: 64 (cosibelimab);
i) the CDR-H1, CDR-H2 and CDR-H3 sequences as comprised in SEQ ID NO: 65, and the CDR-L1, CDR-L2 and CDR-L3 sequences as comprised in SEQ ID NO: 66 (margetuximab);
j) the CDR-H1, CDR-H2 and CDR-H3 sequences as comprised in SEQ ID NO: 67, and the CDR-L1, CDR-L2 and CDR-L3 sequences as comprised in SEQ ID NO: 68 (pertuzumab);
k) the CDR-H1, CDR-H2 and CDR-H3 sequences as comprised in SEQ ID NO: 69, and the CDR-L1, CDR-L2 and CDR-L3 sequences as comprised in SEQ ID NO: 70 (enoblituzumab);
l) the CDR-H1, CDR-H2 and CDR-H3 sequences as comprised in SEQ ID NO: 71, and the CDR-L1, CDR-L2 and CDR-L3 sequences as comprised in SEQ ID NO: 72 (necitumumab);
m) the CDR-H1, CDR-H2 and CDR-H3 sequences as comprised in SEQ ID NO: 73, and the CDR-L1, CDR-L2 and CDR-L3 sequences as comprised in SEQ ID NO: 74 (panitumumab);
n) the CDR-H1, CDR-H2 and CDR-H3 sequences as comprised in SEQ ID NO: 75, and the CDR-L1, CDR-L2 and CDR-L3 sequences as comprised in SEQ ID NO: 76 (amivantamab EGFR-binding);
o) the CDR-H1, CDR-H2 and CDR-H3 sequences as comprised in SEQ ID NO: 77, and the CDR-L1, CDR-L2 and CDR-L3 sequences as comprised in SEQ ID NO: 78 (amivantamab cMet-binding);
p) the CDR-H1, CDR-H2 and CDR-H3 sequences as comprised in SEQ ID NO: 79, and the CDR-L1, CDR-L2 and CDR-L3 sequences as comprised in SEQ ID NO: 80 (zolbetuximab);
q) the CDR-H1, CDR-H2 and CDR-H3 sequences as comprised in SEQ ID NO: 81, and the CDR-L1, CDR-L2 and CDR-L3 sequences as comprised in SEQ ID NO: 82 (dinutuximab);
r) the CDR-H1, CDR-H2 and CDR-H3 sequences as comprised in SEQ ID NO: 83, and the CDR-L1, CDR-L2 and CDR-L3 sequences as comprised in SEQ ID NO: 84 (naxitamab);
s) the CDR-H1, CDR-H2 and CDR-H3 sequences as comprised in SEQ ID NO: 85, and the CDR-L1, CDR-L2 and CDR-L3 sequences as comprised in SEQ ID NO: 86 (enfortumab);
t) the CDR-H1, CDR-H2 and CDR-H3 sequences as comprised in SEQ ID NO: 87, and the CDR-L1, CDR-L2 and CDR-L3 sequences as comprised in SEQ ID NO: 88 (farletuzumab);
u) the CDR-H1, CDR-H2 and CDR-H3 sequences as comprised in SEQ ID NO: 89, and the CDR-L1, CDR-L2 and CDR-L3 sequences as comprised in SEQ ID NO: 90 (tisotumab);
v) the CDR-H1, CDR-H2 and CDR-H3 sequences as comprised in SEQ ID NO: 91, and the CDR-L1, CDR-L2 and CDR-L3 sequences as comprised in SEQ ID NO: 92 (mirvetuximab);
w) the CDR-H1, CDR-H2 and CDR-H3 sequences as comprised in SEQ ID NO: 93, and the CDR-L1, CDR-L2 and CDR-L3 sequences as comprised in SEQ ID NO: 94 (sacituzumab);
x) the CDR-H1, CDR-H2 and CDR-H3 sequences as comprised in SEQ ID NO: 95, and the CDR-L1, CDR-L2 and CDR-L3 sequences as comprised in SEQ ID NO: 96 (vobramitamab);
y) the CDR-H1, CDR-H2 and CDR-H3 sequences as comprised in SEQ ID NO: 97, and the CDR-L1, CDR-L2 and CDR-L3 sequences as comprised in SEQ ID NO: 98 (Onartuzumab);
z) the CDR-H1, CDR-H2 and CDR-H3 sequences as comprised in SEQ ID NO: 144, and the CDR-L1, CDR-L2 and CDR-L3 sequences as comprised in SEQ ID NO: 145 (sibrotuzumab);
aa) the CDR-H1, CDR-H2 and CDR-H3 sequences as comprised in SEQ ID NO: 100, and the CDR-L1, CDR-L2 and CDR-L3 sequences as comprised in SEQ ID NO: 101 (olaratumab);
ab) the CDR-H1, CDR-H2 and CDR-H3 sequences as comprised in SEQ ID NO: 102, and the CDR-L1, CDR-L2 and CDR-L3 sequences as comprised in SEQ ID NO: 103 (rovalpituzumab); and,
ac) the CDR-H1, CDR-H2 and CDR-H3 sequences as comprised in SEQ ID NO: 177, and the CDR-L1, CDR-L2 and CDR-L3 sequences as comprised in SEQ ID NO: 179.
   10. A multispecific antigen binding protein according to embodiment 9, wherein at least one of the first and third antigen-binding region comprises a combination of variable light (V_{L}) and variable heavy (V_{H}) domains selected from the group consisting of:
a) the V_{H} sequence as comprised in SEQ ID NO: 1 and the V_{L} sequence as comprised in SEQ ID NO: 2;
b) the V_{H} sequence as comprised in SEQ ID NO: 3 and the V_{L} sequence as comprised in SEQ ID NO: 4;
c) the V_{H} sequence as comprised in SEQ ID NO: 5 and the V_{L} sequence as comprised in SEQ ID NO: 6;
d) the V_{H} sequence as comprised in SEQ ID NO: 7 and the V_{L} sequence as comprised in SEQ ID NO: 8;
e) the V_{H} sequence as comprised in SEQ ID NO: 9 and the V_{L} sequence as comprised in SEQ ID NO: 10
f) the V_{H} sequence as comprised in SEQ ID NO: 59 and the V_{L} sequence as comprised in SEQ ID NO: 60 (atezolizumab);
g) the V_{H} sequence as comprised in SEQ ID NO: 61 and the V_{L} sequence as comprised in SEQ ID NO: 62 (durvalumab);
h) the V_{H} sequence as comprised in SEQ ID NO: 63 and the V_{L} sequence as comprised in SEQ ID NO: 64 (cosibelimab);
i) the V_{H} sequence as comprised in SEQ ID NO: 65 and the V_{L} sequence as comprised in SEQ ID NO: 66 (margetuximab);
j) the V_{H} sequence as comprised in SEQ ID NO: 67 and the V_{L} sequence as comprised in SEQ ID NO: 68 (pertuzumab);
k) the V_{H} sequence as comprised in SEQ ID NO: 69 and the V_{L} sequence as comprised in SEQ ID NO: 70 (enoblituzumab);
l) the V_{H} sequence as comprised in SEQ ID NO: 71 and the V_{L} sequence as comprised in SEQ ID NO: 72 (necitumumab);
m) the V_{H} sequence as comprised in SEQ ID NO: 73 and the V_{L} sequence as comprised in SEQ ID NO: 74 (panitumumab);
n) the V_{H} sequence as comprised in SEQ ID NO: 75 and the V_{L} sequence as comprised in SEQ ID NO: 76 (amivantamab EGFR-binding);
o) the V_{H} sequence as comprised in SEQ ID NO: 77 and the V_{L} sequence as comprised in SEQ ID NO: 78 (amivantamab cMet-binding);
p) the V_{H} sequence as comprised in SEQ ID NO: 79 and the V_{L} sequence as comprised in SEQ ID NO: 80 (zolbetuximab);
q) the V_{H} sequence as comprised in SEQ ID NO: 81 and the V_{L} sequence as comprised in SEQ ID NO: 82 (dinutuximab);
r) the V_{H} sequence as comprised in SEQ ID NO: 83 and the V_{L} sequence as comprised in SEQ ID NO: 84 (naxitamab);
s) the V_{H} sequence as comprised in SEQ ID NO: 85 and the V_{L} sequence as comprised in SEQ ID NO: 86 (enfortumab);
t) the V_{H} sequence as comprised in SEQ ID NO: 87 and the V_{L} sequence as comprised in SEQ ID NO: 88 (farletuzumab);
u) the V_{H} sequence as comprised in SEQ ID NO: 89 and the V_{L} sequence as comprised in SEQ ID NO: 90 (tisotumab);
v) the V_{H} sequence as comprised in SEQ ID NO: 91 and the V_{L} sequence as comprised in SEQ ID NO: 92 (mirvetuximab);
w) the V_{H} sequence as comprised in SEQ ID NO: 93 and the V_{L} sequence as comprised in SEQ ID NO: 94 (sacituzumab);
x) the V_{H} sequence as comprised in SEQ ID NO: 95 and the V_{L} sequence as comprised in SEQ ID NO: 96 (vobramitamab);
y) the V_{H} sequence as comprised in SEQ ID NO: 97 and the V_{L} sequence as comprised in SEQ ID NO: 98 (onartuzumab);
z) the V_{H} sequence as comprised in SEQ ID NO: 144 and the V_{L} sequence as comprised in SEQ ID NO: 145 (sibrotuzumab);
aa) the V_{H} sequence as comprised in SEQ ID NO: 100 and the V_{L} sequence as comprised in SEQ ID NO: 101 (olaratumab);
ab) the V_{H} sequence as comprised in SEQ ID NO: 102 and the V_{L} sequence as comprised in SEQ ID NO: 103 (rovalpituzumab); and,
ac) the CDR-H1, CDR-H2 and CDR-H3 sequences as comprised in SEQ ID NO: 177, and the CDR-L1, CDR-L2 and CDR-L3 sequences as comprised in SEQ ID NO: 179.
   11. A multispecific antigen binding protein according to any one of the preceding embodiments, wherein the second antigen-binding region comprises or consists of i) an immunoglobulin Fc region or ii) an antigen-binding region that specifically binds a surface antigen expressed on NK cells, wherein preferably, the surface antigen expressed on NK cells is an NK cell activating receptor.
   12. A multispecific antigen binding protein according to embodiment 11, wherein the Fc region is a dimeric Fc region.
   13. A multispecific antigen binding protein according to embodiment 11 or 12, wherein the Fc region binds to CD16A.
   14. A multispecific antigen binding protein according to embodiment 13, wherein the Fc region is modified to reduce or enhance affinity for CD16A, relative to a corresponding wild-type Fc region.
   15. A multispecific antigen binding protein according to embodiment 13, wherein the Fc region is modified to reduce or enhance NK cell activation through CD16A binding, relative to a corresponding wild-type Fc region.
   16. A multispecific antigen binding protein according to embodiment 5 or 11, wherein the NK cell activating receptor selected from the group consisting of: NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, NTB-A, CRACC, 4-1BB, OX40, CRTAM, CD27, PSGL1, CD96, CD100, CD59, PD-L1, Tim3 and CEACAM1.
   17. A multispecific antigen binding protein according to embodiment 16, wherein the second or third antigen-binding region activates the NK cell activating receptor.
   18. A multispecific antigen binding protein according to any one of the preceding embodiments, wherein the IL21R agonist comprises or consist of an IL21 polypeptide or an agonistic antigen-binding region that specifically binds IL21R.
   19. A multispecific antigen binding protein according to embodiment 18, wherein the IL21 polypeptide comprises an amino acid sequence with at least 50, 55, 60, 65, 70, 75, 80, 85, 90, 95, 96, 97, 98, 99, or 100% sequence identity to SEQ ID NO: 38, and preferably has at least one of IL21R agonist activity and affinity for the IL21R.
   20. A multispecific antigen binding protein according to embodiment 18 or 19, wherein the IL21 polypeptide is an IL21 mutein that is modified to reduce or enhance affinity for IL21R, relative to a corresponding wild type IL21 polypeptide.
   21. A multispecific antigen binding protein according to embodiment 20, wherein the IL21 mutein has reduced affinity for IL21R, relative to a corresponding wild type IL21 polypeptide, and wherein the IL21 mutein has a mutation in one or more amino acids selected from the group consisting of 116, 166, 18, K72, K73, K75, K77, L13, P78, Q12, Q19, R5, R65, R76, R9, S70, S80, V69 and Y23.
   22. A multispecific antigen binding protein according to any one of embodiments 18-21, wherein the multispecific antigen binding protein has an IL21R agonist-valency that is higher than one.
   23. A multispecific antigen binding protein according to any one of the preceding embodiments, wherein the 4-1BB agonist comprises or consist of at least one 4-1BB ligand (4-1BBL) extracellular domain (ECD) or at least one agonistic antigen-binding region that specifically binds 4-1 BB.
   23. A multispecific antigen binding protein according to embodiment 22, wherein the 4-1BBL ECD comprises an amino acid sequence with at least 50, 55, 60, 65, 70, 75, 80, 85, 90, 95, 96, 97, 98, 99, or 100% sequence identity to SEQ ID NO: 37, and preferably has at least one of 4-1 BB agonist activity and affinity for 4-1 BB.
   24. A multispecific antigen binding protein according to embodiment 22 or 23, wherein the 4-1BBL ECD is a mutein that is modified to reduce, enhance affinity, improve stability or improve expression for 4-1 BB, relative to a corresponding wild type 4-1 BBL ECD.
   25. A multispecific antigen binding protein according to any one of embodiments 22-24, wherein the 4-1 BB agonist comprises or consist of a fusion protein comprising three 4-1 BBL ECD monomers fused together in a single polypeptide chain, and wherein, optionally, the three 4-1 BBL ECD monomers are connected by polypeptide linkers.
   26. A multispecific antigen binding protein according to any one of embodiments 22-25, wherein the multispecific antigen binding protein has a 4-1 BB agonist-valency that is higher than one.
   27. A multispecific antigen binding protein according to any one of embodiments 18-26, wherein the multispecific antigen binding protein comprises an IL21R agonist and a 4-1 BB agonist.
   28. A multispecific antigen binding protein according to any one of the preceding embodiments, wherein the multispecific antigen binding protein further comprises an NK cell-activating cytokine selected from an IL15 receptor agonist, a type I interferon (IFN-1) agonist, an IL2 receptor agonist, an IL12 receptor agonist and an IL18 receptor agonist.
   29. A multispecific antigen binding protein according to any one of the preceding embodiments, wherein at least one of the first and third antigen-binding regions that specifically binds a TAA is conjugated to the second antigen-binding region that has affinity for a surface antigen expressed on NK cells.
   30. A multispecific antigen binding protein according to embodiment 29, wherein at least one polypeptide chain in the at least one of the first and third antigen-binding regions forms a single polypeptide chain with at least one polypeptide chain of the second antigen-binding region.
   31. A multispecific antigen binding protein according to embodiment 30, wherein the single polypeptide chain comprises in an N- to C-terminal order: i) at least one polypeptide chain in the at least one of the first and third antigen-binding region; ii) optionally a flexible linker; and iii) the second antigen-binding region.
   32. A multispecific antigen binding protein according to embodiment 30 or 31, wherein the second antigen-binding region is a dimeric Fc region, wherein each of the two polypeptide chains of the dimeric Fc region is linked to a CH1 domain, each of which CH1 domains is linked to an immunoglobulin-derived antigen-binding region that specifically binds a TAA.
   33. A multispecific antigen binding protein according to embodiment 32, wherein the two immunoglobulin-derived antigen-binding regions bind the same TAA, or wherein the two immunoglobulin-derived antigen-binding region each bind a different TAA.
   34. A multispecific antigen binding protein according to embodiment 32 or 33, wherein the protein comprises a dimeric Fc region, wherein each of the two Fc polypeptide chains is operably linked to a Fab that specifically binds a TAA.
   35. A multispecific antigen binding protein according to any one of embodiments 29-34, wherein at least one of the NK cell-activating cytokines, is conjugated to the at least one antigen-binding region that specifically binds a TAA, or to the second antigen-binding region.
   36. A multispecific antigen binding protein according to embodiment 35, wherein at least one of the NK cell-activating cytokines forms a single polypeptide chain with at least one of:
i) at least one polypeptide chain in at least one of the first and third antigen-binding regions; and,
ii) at least one polypeptide chain in the second antigen-binding region;
wherein optionally, a flexible linker is present between the agonist and the at least one polypeptide chain in the region defined in i) or ii).
   37. A multispecific antigen binding protein according to any one of embodiments 34-36, wherein at least one of the NK cell-activating cytokines forms a single polypeptide chain with at least one of:
i) a light chain in at least one of the two Fabs that specifically bind a TAA; and,
ii) at least one of the two Fc chains in the dimeric Fc region;
wherein optionally, a flexible linker is present between the agonist and the light chain defined in i) or the Fc chain defined in ii).
   38. A multispecific antigen binding protein according to embodiment 37, wherein at least one of the NK cell-activating cytokines is fused to at least one of:
i) the N-terminus of the light chain in at least one of the two Fab's that specifically bind a TAA, optionally through a flexible linker;
ii) the C-terminus of the light chain in at least one of the two Fab's that specifically bind a TAA, optionally through a flexible linker;
iii) the N-terminus of the heavy chain in at least one of the two Fab's that specifically bind a TAA; and,
iv) the C-terminus of the heavy chain in at least one of the two Fc chains in the dimeric immunoglobulin Fc domain, optionally through a flexible linker.
   39. A multispecific antigen binding protein according to embodiment 37 or 38, wherein at least one of the NK cell-activating cytokines is present on at least one or on both sides of the immunoglobulin structure.
   40. A multispecific antigen binding protein according to any one of embodiments 32-39, wherein the multispecific antigen binding protein is heterodimeric with respect to at least one of i) the first and third antigen-binding regions; and ii) at least one fused NK cell-activating cytokine, and wherein the dimeric Fc region comprises different first and a second polypeptide chains comprising knob-into-hole modifications promoting association of the first and the second polypeptide chains of the Fc region.
   41. A multispecific antigen binding protein according to any one of the preceding embodiments, wherein the multispecific antigen binding protein has at least one biological activity selected from:
a) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector: target cell ratio, with the same NK cells and target cells that are not brought into contact with the multispecific antigen binding protein; and,
b) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector: target cell ratio, with the same NK cells and target cells that are brought into contact with a conventional human IgG1 monoclonal antibody that has the same TAA-specific antigen-binding regions as the multispecific antigen binding protein.
   42. A multispecific antigen binding protein according to any one of the preceding embodiments, wherein ex vivo expansion of donor NK cells by co-culturing with the multispecific antigen binding protein as described herein, produces a population of expanded NK cells having one or more features selected from:
a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells);
b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells;
c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells;
d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and,
e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells,
wherein, preferably the NK cells are co-cultured with tumor cells expressing a TAA specifically bound by the multispecific antigen binding protein.
   43. A pharmaceutical composition comprising a multispecific antigen binding protein according to any one of the preceding embodiments, and a pharmaceutically acceptable carrier.
   44. An ex vivo method for expansion of NK cells, the method comprising the step of contacting an NK cell with a multispecific antigen binding protein according to any one of embodiments 1-42, or with a composition according to embodiment 42, and wherein preferably, the expanded NK cells have one or more features selected from:
a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells);
b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells;
c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells;
d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and,
e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells, wherein, preferably the method comprising the further step of co-culturing the NK cells with tumor cells expressing a TAA specifically bound by the multispecific antigen binding protein.
   45. A multispecific antigen binding protein according to any one of embodiments 1-42, a composition according to embodiment 42, or an ex vivo expanded NK cell obtained in a method according to embodiment 43, optionally in combination with the multispecific antigen binding protein, for use as a medicament.
   46. A multispecific antigen binding protein according to any one of embodiments 1-42, a composition according to embodiment 42, or an ex vivo expanded NK cell obtained in a method according to embodiment 43, optionally, in combination with the multispecific antigen binding protein, for use in the treatment of a cancer, preferably a cancer comprising tumor cells expressing the TAA.
   47. A multispecific antigen binding protein according to any one of embodiments 1-42, a composition according to embodiment 42, for use in the treatment of a cancer, preferably a cancer comprising tumor cells expressing the TAA, wherein the multispecific antigen binding protein or the composition is used in combination with an adoptive transfer of immune cells, wherein preferably the immune cells are selected from T cells and NK cells.
   48. A multispecific antigen binding protein according to any one of embodiments 1-42, a composition according to embodiment 42, for a use according to claim 46 or 47, wherein at least one of:
a) the multispecific antigen binding protein is administered as a neoadjuvant therapy before a primary therapy comprising at least one of surgery and radiation therapy of the cancer; and,
b) the multispecific antigen binding protein is administered as an adjuvant therapy after a primary therapy comprising at least one of surgery and radiation therapy of the cancer.
   49. A method for enhancing anti-tumor activity of an NK cell in a subject, the method comprising the step of administering to the subject a multispecific antigen binding protein according to any one of embodiments 1-42, a composition according to embodiment 42, an ex vivo expanded NK cell obtained in a method according to embodiment 43, optionally, in combination with the multispecific antigen binding protein, or a combination of the multispecific antigen binding protein and an immune cell selected from T cells and NK cells.
   50. The method of 49, wherein the subject has cancer, preferably a cancer comprising tumor cells expressing the TAA.
   51. The method of claim 50, wherein at least one of:
a) the multispecific antigen binding protein is administered to the subject as a neoadjuvant therapy before a primary therapy comprising at least one of surgery and radiation therapy of the cancer; and,
b) the multispecific antigen binding protein is administered to the subject as an adjuvant therapy after a primary therapy comprising at least one of surgery and radiation therapy of the cancer.
   52. A nucleic acid molecule comprising one or more nucleotide sequences encoding a polypeptide chain of a multispecific antigen binding protein according to any one of embodiments 1-42.
   53. A nucleic acid molecule according to embodiment 52, wherein the one or more nucleotide sequences are operably linked to regulatory sequences for expression of the one or more polypeptide chains in a host cell.
   54. A host cell comprising a nucleic acid molecule according to embodiment 52 or 53.
   55. A method for producing a multispecific antigen binding protein according to any one of embodiments 1-42, the method comprising culturing a host cell according to embodiment 54 such that one or more nucleotide sequences are expressed, and the multispecific antigen binding protein is produced.
   56. A method according to embodiment 55, further comprising the steps of: recovery of the multispecific antigen binding protein, and, optionally, formulation of the multispecific antigen binding protein with a pharmaceutically acceptable carrier.

1. A multispecific antigen binding protein comprising:
a) a first antigen-binding region that specifically binds a tumor associated antigen (TAA);
b) a second antigen-binding region that has affinity for a surface antigen expressed on natural killer (NK) cells, wherein the second antigen-binding region comprises or consists of an immunoglobulin Fc region; and,
c) at least two NK cell-activating cytokines comprising:
i) an interleukin 21 receptor (IL21R) agonist; and,
ii) a 4-1BB agonist,
wherein the IL21R agonist comprises or consist of an IL21 polypeptide or an agonistic antigen-binding region that specifically binds IL21R, and wherein the 4-1BB agonist comprises or consist of at least one 4-1BB ligand (4-1BBL) extracellular domain (ECD) or at least one agonistic antigen-binding region that specifically binds 4-1BB.

2. A multispecific antigen binding protein according claim 1, wherein the IL21 polypeptide comprises an amino acid sequence with at least 70% sequence identity to SEQ ID NO: 38 and having IL21R agonist activity, and wherein the 4-1BBL ECD comprises an amino acid sequence with at least 70% sequence identity to SEQ ID NO: 37 and having 4-1BB agonist activity.

3. A multispecific antigen binding protein according claim 1, wherein the TAA is selected from the group consisting of: Her2 (ErbB2/Neu), Receptor Tyrosine Kinase-like Orphan Receptor 1 (ROR1), Crypto, CD2, CD4, CD20, CD30, CD19, CD38, CD40, CD47, Glycoprotein NMB, CanAg, CD22 (Siglec2), CD33 (Siglec3), CD79, CD123, CD138, CD171, CTLA-4 (CD152), PD1, PSCA, L1-CAM, EpCAM, PSMA (prostate specific membrane antigen), BCMA, TROP2, STEAP1, CD52, CD56, CD80, CD70, E-selectin, EphB2, EPHA4, Melanotransferrin, Mud 6, TMEFF2, Killer 1g-Like Receptor, Killer 1g-Like Receptor 3DL2 (KIR3DL2), B7.1, B7.2, B7-H3, B7-H4, B7-H6, PD-L1, IL-6 receptor, IL-1 accessory Protein, MAGE, MART-1/Melan-A, gp100, MICA, MICB, adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), NaPi2b, TYRP1, nectin-4, a UL16-binding protein (ULBP), a RAET1 protein, carcinoembryonic antigen (CEA), CEACAM5, etv6, aml1, prostate specific antigen (PSA), T-cell receptor/CD3-zeta chain, MAGE-A3, a GAGE-tumor antigen, anti-Müllerian hormone Type II receptor, delta-like ligand 3 (DLL3), delta-like ligand 4 (DLL4), DR5, NTRKR1 (EC 2.7.10.1), SLAMF7, TRAILR1, TRAILR2, BAGE, RAGE, LAGE-1, NAG, GnT-V, MUM-1, CDK4, MUC1, MUC1-C, VEGF, VEGFR2, Angiopoietin-2, PDGF, TGF-alpha, EGF, EGF receptor (EGFR/ERBB1), HER-3/ERBB3, HER-4/ERBB4, a heterodimeric receptor comprised of at least one HER subunit, gastrin releasing peptide receptor antigen, cMET, CA125, integrin receptors, a5β3 integrins, a5β1 integrins, αllbβ3-integrins, PDGF alpha receptor, PDGF beta receptor, sVE-cadherin, IL-8 receptor, hCG, IL-6 receptor, IL-1 accessory protein, CSF1R, α-fetoprotein, mesothelin (MSLN), Isoform 2 of Claudin-18 (Claudin 18.2, CLDN18), folate receptor alpha (FRα, FOLR1), tissue factor (TF, CD142), P-cadherin, E-cadherin, α-catenin, β-catenin and γ-catenin, Plexin-A1, TNFRSF10B, AXL, EDNRB, OLR1, ADAM12, PLAUR, CCR4, CCR6, p120ctn, PRAME, NY-ESO-1, cdc27, CDCP1, adenomatous polyposis coli protein (APC), fodrin, Connexin 37, Ig-idiotype, p15, gp75, a GM2 ganglioside, a GD2 ganglioside, a human papillomavirus protein, imp-1, P1A, EBV-encoded nuclear antigen (EBNA)-I, brain glycogen phosphorylase, SSX-1, SSX-2 (HOM-MEL-40), SSX-1, SSX-4, SSX-5, SCP-1 CT-7, c-erbB-2, FcRL5/FcRH5, Flt3, muci6, muci7, mmp9, FAP, Lewis-Y, EGFRvIII, GPC3, GPRC5D, gpA33, 5T4, SSTR2, CD73, CD25, CD45, and CD133, and wherein preferably, the multispecific antigen binding protein comprises a third antigen-binding region that specifically binds a TAA and wherein the first and third antigen-binding regions can bind the same TAA or at least two different TAAs.

4. A multispecific antigen binding protein according to claim 1, wherein the Fc region is a dimeric Fc region that binds to CD16A and, preferably activates the NK cell.

5. A multispecific antigen binding protein according to claim 1, wherein the 4-1BB agonist comprises or consist of a fusion protein comprising three 4-1BBL ECD monomers fused together in a single polypeptide chain, and wherein, optionally, the three 4-1BBL ECD monomers are connected by polypeptide linkers.

6. A multispecific antigen binding protein according to claim 1, wherein at least one polypeptide chain in the at least one of the first and third antigen-binding regions forms a single polypeptide chain with at least one polypeptide chain of the second antigen-binding region, wherein preferably, the single polypeptide chain comprises in an N- to C-terminal order: i) at least one polypeptide chain in the at least one of the first and third antigen-binding region; ii) optionally a flexible linker; and iii) the second antigen-binding region, wherein more preferably the second antigen-binding region is a dimeric Fc region, wherein each of the two polypeptide chains of the dimeric Fc region is linked to a CH1 domain, each of which CH1 domains is linked to an immunoglobulin-derived antigen-binding region that specifically binds a TAA, wherein most preferably, the protein comprises a dimeric Fc region, wherein each of the two Fc polypeptide chains is operably linked to a Fab that specifically binds a TAA.

7. A multispecific antigen binding protein according to claim 6, wherein at least one of the NK cell-activating cytokines, is conjugated to the at least one antigen-binding region that specifically binds a TAA, or to the second antigen-binding region,
wherein preferably, at least one of the NK cell-activating cytokines forms a single polypeptide chain with at least one of:
i) at least one polypeptide chain in at least one of the first and third antigen-binding regions; and,
ii) at least one polypeptide chain in the second antigen-binding region;
wherein optionally, a flexible linker is present between the agonist and the at least one polypeptide chain in the region defined in i) or ii), wherein more preferably, wherein at least one of the NK cell-activating cytokines forms a single polypeptide chain with at least one of:
i) a light chain in at least one of the two Fab's that specifically bind a TAA; and,
ii) at least one of the two Fc chains in the dimeric Fc region;
wherein optionally, a flexible linker is present between the agonist and the light chain defined in i) or the Fc chain defined in ii),
wherein more preferably, at least one of the NK cell-activating cytokines is fused to at least one of:
i) the N-terminus of the light chain in at least one of the two Fab's that specifically bind a TAA, optionally through a flexible linker;
ii) the C-terminus of the light chain in at least one of the two Fab's that specifically bind a TAA, optionally through a flexible linker;
iii) the N-terminus of the heavy chain in at least one of the two Fab's that specifically bind a TAA; and,
iv) the C-terminus of the heavy chain in at least one of the two Fc chains in the dimeric immunoglobulin Fc domain, optionally through a flexible linker
wherein most preferably, at least one of the NK cell-activating cytokines is present on at least one or on both sides of the immunoglobulin structure.

8. A multispecific antigen binding protein according to claim 6, wherein the multispecific antigen binding protein is heterodimeric with respect to at least one of i) the first and third antigen-binding regions; and ii) at least one fused NK cell-activating cytokine, and wherein the dimeric Fc region comprises different first and a second polypeptide chains comprising knob-into-hole modifications promoting association of the first and the second polypeptide chains of the Fc region.

9. A multispecific antigen binding protein according to claim 1, wherein the multispecific antigen binding protein has at least one biological activity selected from:
a) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are not brought into contact with the multispecific antigen binding protein; and,
b) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are brought into contact with a conventional human IgG1 monoclonal antibody that has the same TAA-specific antigen-binding regions as the multispecific antigen binding protein.

10. A multispecific antigen binding protein according to claim 1, wherein ex vivo expansion of donor NK cells by co-culturing with the multispecific antigen binding protein, produces a population of expanded NK cells having one or more features selected from:
a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells);
b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells;
c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells;
d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and,
e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells,
wherein, preferably the NK cells are co-cultured with tumor cells expressing a TAA specifically bound by the multispecific antigen binding protein.

11. (canceled)

12. An ex vivo method for expansion of NK cells, the method comprising the step of contacting an NK cell with a multispecific antigen binding protein according to a claim 1, and wherein preferably, the expanded NK cells have one or more features selected from:
a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells);
b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells;
c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells;
d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and,
e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells,
wherein, preferably the method comprising the further step of co-culturing the NK cells with tumor cells expressing a TAA specifically bound by the multispecific antigen binding protein.

13-15. (canceled)

16. A method for treating cancer in a subject in need thereof, the method comprising administering the multispecific antigen binding protein according to claim 1 to the subject.

17. The method according to claim 16, wherein the treatment is for a cancer comprising tumor cells expressing the TAA.

18. The method according to claim 16, wherein the multispecific antigen binding protein is used in combination with an adoptive transfer of immune cells, wherein preferably the immune cells are selected from T cells and NK cells.

19. The method according to claim 16, wherein at least one of:
a) the multispecific antigen binding protein and/or the ex vivo expanded NK cell is administered as a neoadjuvant therapy before a primary therapy comprising at least one of surgery and radiation therapy of the cancer; and,
b) the multispecific antigen binding protein and/or the ex vivo expanded NK cell is administered as an adjuvant therapy after a primary therapy comprising at least one of surgery and radiation therapy of the cancer.
